(19) United States
(12) Patent Application Publication
(10) Pub. No.: US 2026/0115298 A1
(43) Pub. Date: Apr. 30, 2026

(54) PTH Treatment Regimen Comprising Two PTH Compounds

(71) Applicant: Ascendis Pharma Bone Diseases A/S, Hellerup (DK)

(72) Inventors: Kennett SPROGØE, Hellerup (DK); Thomas William ANDERSON, Hellerup (DK)

(21) Appl. No.: 19/120,650

(22) PCT Filed: Oct. 31, 2023
(86) PCT No.: PCT/EP2023/080318

§ 371 Date: Apr. 11, 2025

(30) Foreign Application Priority Data
Nov. 2, 2022 (EP) 22205058.5

(51) Int. Cl. A61K 47/60; A61K 9/00; A61K 38/29; A61P 5/20; G01N 33/84

(52) U.S. Cl. CPC A61K 47/60; A61K 9/0019; A61K 38/29; A61P 5/20; G01N 33/84; G01N 2800/52

(57) ABSTRACT

The present invention relates to a PTH compound for use in the treatment of chronic hypoparathyroidism, wherein the treatment comprises the step of administering to a patient having chronic hypoparathyroidism a once weekly dose of a PTH compound and wherein prior to initiation of the treatment the patient is clinically determined as being no longer dependent on active vitamin D and calcium supplement to maintain serum within normal range; and related aspects.

The present invention relates to a PTH compound for use in the treatment of chronic hypoparathyroidism, wherein the treatment comprises the step of administering to a patient having chronic hypoparathyroidism a once weekly dose of a PTH compound and wherein prior to initiation of the treatment the patient is clinically determined as being no longer dependent on active vitamin D and calcium supplement to maintain serum within normal range; and related aspects.

Hypoparathyroidism is a rare endocrine disease with low serum calcium and inappropriately low (insufficient) circulating parathyroid hormone (PTH) levels, most often in adults secondary to thyroid surgery. Standard treatment, also referred to as conventional therapy, is active vitamin D and calcium supplement, which increases calcium and phosphorus absorption and serum levels at the expense of abnormally increased urinary calcium excretion.

A major complication of hypoparathyroidism is hypercalciuria, due to the lack of PTH dependent calcium reabsorption in the distal renal tubules. Hypercalciuria is associated with an increased risk of nephrocalcinosis, nephrolithiasis and kidney failure. Furthermore, unphysiological levels of PTH may lead to hypocalcemia, if PTH levels are too low, and hypercalcemia, if PTH levels are too high, such as due to an overdose of externally administered PTH. Hypocalcemia may lead to symptoms such as numbness, muscle spasms, seizures, confusion, or cardiac arrest. Symptoms of hypercalcemia may include abdominal pain, bone pain, confusion, depression, weakness, kidney stones or an abnormal heart rhythm including cardiac arrest.

Physiological actions of PTH include releasing calcium and phosphorus from bone, retaining calcium but not phosphorus in the kidney, and stimulating the renal production of active vitamin D (1,25(OH)₂vitamin D3), which in turn enhances intestinal calcium and phosphorus absorption. When PTH is delivered intermittently, such as by current daily or multiple daily injections of PTH 1-84 or PTH 1-34, it acts on bone as an anabolic agent by preferentially activating osteoblasts over osteoclasts. This anabolic effect of intermittent PTH exposure may not be beneficial in patients with hypoparathyroidism as the disease is characterized by a state of low bone turnover, and thus already high bone mineral density. In contrast, continuous exposure to PTH may provide more normal bone turnover and restore physiologically normal skeleton.

Several PTH therapies are currently in development that aim for once daily (long-acting) or weekly (ultra-long-acting) administration with a dose that effectively maintains serum calcium within normal levels. Titrating patients off of conventional therapy and switching them to a long-acting or ultra-long-acting PTH compound poses the risk of PTH over- or underdosing, which may result in hyper- or hypocalcemia with the associated negative side-effects, respectively. Therefore, to provide a safe treatment this titration process would have to be done by starting with a low and safe dose of the long-acting or ultra-long-acting PTH compound in combination with conventional therapy, followed by multiple dose adjustments, in which administration of conventional therapy is ideally eliminated and ultimately the safe and effective dose of the long-acting or ultra-long-acting PTH compound identified. Although this may be a safe process, it is also slow, and during the long titration period the patient does not benefit from a true and effective replacement therapy.

For a PTH product indicated for adjunct therapy to active vitamin D and calcium, severe hypercalcemia has been reported. In the pivotal trial, 3 patients randomized to PTH therapy required administration of IV fluids to correct hypercalcemia.

Especially with weekly or longer administration intervals this titration process will take a significant amount of time, because an overdosing of, for example, a weekly PTH compound will require a significantly longer time period for the PTH to be cleared from the patient's body and thus has the potential to lead to adverse effects lasting for an unacceptable period of time.

Thus, although therapy with an ultra-long-acting PTH compound appears attractive from a patient's perspective, it poses new and so far unidentified challenges, for which there are currently no solutions.

In a first aspect the present invention relates to a PTH compound for use in the treatment of chronic hypoparathyroidism, wherein the treatment comprises the step of administering to a patient having chronic hypoparathyroidism a once weekly dose of a PTH compound and wherein prior to initiation of the treatment the patient is clinically determined as being no longer dependent on active vitamin D and calcium supplement to maintain serum within normal range.

In a second aspect the present invention relates to a method of treating a patient having chronic hypoparathyroidism, the method comprising the step of administering to the patient a pharmaceutically effective once weekly dose of a PTH compound, wherein prior to the initiation of the treatment said patient is clinically determined as being no longer dependent on active vitamin D and calcium supplement to maintain serum calcium within normal range.

In a third aspect the present invention relates to a PTH compound for use in the manufacture of a medicament for the treatment of chronic hypoparathyroidism, wherein the medicament is administered to a patient having chronic hypoparathyroidism once weekly and wherein prior to administration of the first dose of the medicament the patient has been clinically determined as being no longer dependent on active vitamin D and calcium supplement to maintain serum calcium within normal range.

In a fourth aspect the present invention relates to a first PTH compound and a second PTH compound for use in the treatment of chronic hypoparathyroidism, wherein the treatment comprises the step of administering over a first treatment period a daily pharmaceutically effective dose of a first PTH compound, and then switching to administering over a second treatment period a weekly dose of a second PTH compound, wherein the switching of treatment periods occurs after the patient is clinically determined as being no longer dependent on active vitamin D and calcium supplement to maintain serum calcium within normal range.

In a fifth aspect the present invention relates to a method of treating a patient having chronic hypoparathyroidism, the method comprising the step of administering over a first treatment period a daily pharmaceutically effective dose of a first PTH compound, and then switching to administering over a second treatment period a weekly pharmaceutically effective dose of a second PTH compound, wherein the switching of treatment periods occurs after the patient is clinically determined as being no longer dependent on active vitamin D and calcium supplement to maintain serum calcium levels within the normal range.

In a sixth aspect the present invention relates to a first PTH compound for use in the manufacture of a first medicament for the treatment of chronic hypoparathyroidism and a second PTH compound for use in the manufacture of a second medicament for the treatment of chronic hypoparathyroidism, wherein the first medicament is administered daily to a patient having chronic hypoparathyroidism over a first treatment period and wherein said patient is then switched to weekly administration of the second medicament for a second treatment period, wherein the switching of treatment periods occurs after the patient is clinically determined as being no longer dependent on active vitamin D and calcium supplement to maintain serum calcium levels within the normal range.

In a seventh aspect the present invention relates to a first and a second PTH compound for use in a method of treating chronic hypoparathyroidism, the method comprising:
(a) administering to a patient having chronic hypoparathyroidism the first PTH compound on multiple occasions with a first average interval between administrations, adjusting dose if needed, to determine for the patient a dose at which the patient's serum calcium level remains within normal range in the absence of active vitamin D and calcium supplement;
(b) using the dose determined in step (a) to determine an initial dose for the second PTH compound to be administered on multiple occasions to the patient, with a second average interval, longer than the first average interval; and
(c) administering the second PTH compound on multiple occasions with the second average interval between administrations to the patient starting at the initial dose determined in step (b) with adjustment of the dose, if needed, until the patient's serum calcium remains within normal range in the absence of active vitamin D and calcium supplement.

In an eighth aspect the present invention relates to a method of treating a patient having chronic hypoparathyroidism, comprising:
(a) administering to the patient a first PTH compound on multiple occasions with a first average interval between administrations, adjusting dose if needed, to determine for the patient the dose at which the patient's serum calcium level remains within normal range in the absence of active vitamin D and calcium supplement;
(b) using the dose determined in step (a) to determine an initial dose for a second PTH compound to be administered on multiple occasions to the patient, with a second average interval, longer than the first average interval; and
(c) administering the second PTH compound on multiple occasions with the second average interval between administrations to the patient starting at the initial dose determined in step (b) with adjustment of the dose, if needed, until the patient's serum calcium remains within normal range in the absence of active vitamin D and calcium supplement.

In a ninth aspect the present invention relates to a first PTH compound for use in the manufacture of a first medicament for the treatment of chronic hypoparathyroidism and a second PTH compound for use in the manufacture of a second medicament for the treatment of chronic hypoparathyroidism, wherein the first medicament is intended for
(a) administration to a patient having chronic hypoparathyroidism on multiple occasions with a first average interval between administrations, adjusting dose if needed, to determine for the patient the dose at which the patient's serum calcium level remains within normal range in the absence of active vitamin D and calcium supplement;
(b) using the dose determined in step (a) to determine an initial dose for the second medicament to be administered on multiple occasions to the patient, with a second average interval, longer than the first average interval; and
administering the second medicament on multiple occasions with the second average interval between administrations to the patient starting at the initial dose determined in step (b) with adjustment of the dose, if needed, until the patient's serum calcium remains within normal range in the absence of active vitamin D and calcium supplement.

In a tenth aspect the present invention relates to a method of treating a population of patients having chronic hypoparathyroidism, comprising:
a) administering to the population of patients a first PTH compound on multiple occasions with a first average interval between administration, adjusting dose if needed, to determine for individual patients in the population doses at which the patient's serum calcium level remains within normal range in the absence of active vitamin D and calcium supplement;
b) using the doses to determine initial doses for a second PTH compound to be administered at a second average interval longer than the first average interval for those patients, in which a dose of the first PTH compound has been determined which results in the patient's serum calcium level to be within the normal range in the absence of active vitamin D and calcium supplement; and
c) administering the second PTH compound on multiple occasions with the second average interval between administrations to those patients, for which an initial dose of the second PTH has been determined, with adjustment of the dose if needed, until the patient's serum calcium remains within normal range in the absence of active vitamin D and calcium supplement.

It is challenging to safely initiate PTH naive chronic hypoparathyroidism patients on ultra-long acting PTH therapy, such as therapy with the second PTH compound, due to the careful titration required to prevent hypo- and hypercalcemia, and the inability to rapidly react should these adverse effects occur due to the long PK of such compounds.

Applicants surprisingly found that treatment with a PTH compound, such as with the first PTH compound of the present invention, leads to a stabilization of the PTH dose and identified these patients as ideal candidates for switching to a PTH compound with a longer period between two administrations, such as to a once-weekly PTH therapy, because the risk of calcium excursions is low as no titration of conventional therapy is required.

Applicant has furthermore surprisingly found a safe way of treating patients with an ultra-long-acting PTH compound, such as a weekly PTH compound, that provides the benefits of a proper PTH replacement therapy from the start of the therapy: Initiating treatment with a long-acting PTH compound, such as treatment with the first PTH compound, enables titration off of conventional therapy while maintained the ability for rapid intervention if serum calcium exceeds normal levels by adjusting the dose or skipping a dose of the long-acting PTH compound if serum calcium become too high. This provides patients a safe manner to discontinue conventional therapy and identify the individual PTH dose requirement.

Thus, applicant has identified that chronic hypoparathyroidism patients that are stable, meaning that the dose of a PTH compound, such as the first PTH compound of the present invention, administered allows for serum calcium levels in the normal range in the absence of active vitamin D and calcium supplement, are particularly suited for initiation of treatment with an ultra-long acting PTH compound, such as a PTH compound with for example weekly administration intervals.

The advantage of the present treatment is a reduced time until an effective dose is reached for an ultra-long-acting PTH compound, while at the same time ensuring the patient's safety.

Within the present invention the terms are used having the meaning as follows.

As used herein the terms “within normal level” and “within normal range” with regards to serum calcium (sCa) refer to the calcium level ordinarily found in a subject of a given species, sex and age. In humans, a normal serum calcium level usually corresponds to a serum calcium level ranging from 8.3 mg/dL (albumin-adjusted) to 10.6 mg/dL (albumin-adjusted). The term “albumin-adjusted” with regards to serum calcium levels means that the measured serum calcium level is corrected for calcium bound to albumin according to the following formula:

$$albumin - adjusted serum calcium ( mg / dL ) = measured total Ca ( mg / dL ) + 0.8 ( 4. - serum albumin [ g / dL ] ) .$$

As used herein the term “starting dose” refers to the dose of the first PTH compound that is administered to a patient when first initiating the treatment with the first PTH compound, i.e., such patient has not previously received a dose of the first PTH compound. It is understood that the patient may continue on such starting dose for some time, such as for several days, weeks or months or for the full length of the treatment or may titrate the dose up or down in response to certain events, such as the occurrence of hypo- or hypercalcemia.

As used herein the term “average” refers to the simple (equally weighted) arithmetic mean, which can be obtained by summing up all the variables in the data set and dividing the result by the number of variables.

A patient is referred to as having “chronic hypoparathyroidism” if the hypoparathyroidism has persisted for at least six months.

As used herein, the term “stable patient” refers to a patient having normal serum calcium levels, taking a PTH compound, such as a first or second PTH compound, and not taking conventional therapy, i.e. active vitamin D and calcium supplement. The dose of the PTH compound, such as the first or second PTH compound, administered to the stable patient may be adjusted from time to time. It is understood that a stable patient may still take oral calcium of <600 mg/day, which may be required for nutritional reasons, and which is not considered to be a therapeutic amount of calcium.

As used herein, the term “active vitamin D” corresponds to 1,25-dihydroxyvitamin D, also known as calcitriol.

As used herein the term “active PTH” in relation to the first and second PTH compound refers to a PTH-comprising compound released from the first or second PTH compound, respectively, if the first or second PTH compound releases such PTH-comprising compound and refers to the first and/or second PTH compound per se if the first and/or second PTH does not release a PTH-comprising moiety. For example, the conjugates of formula (I), (I-a), (I-a′), (I-b) and (I-b′) release PTH 1-34, in which case PTH 1-34 is the active PTH of compounds (I), (I-a), (I-a′), (I-b) and (I-b′). The compound of formula (II-i) releases the compound of formula (II-ii) and thus the compound of formula (II-ii) is the active PTH of the compound of formula (II-ii).

Similarly, the compound of formula (II-i′) releases the compound of formula (II-ii′) and thus the compound of formula (II-ii′) is the active PTH of the compound of formula (II-ii). In contrast, in certain embodiments the first PTH compound is a PTH 1-34, PTH 1-84 or AZP-3601, which do not release a PTH-comprising compound and thus the active PTH of PTH 1-34, PTH 1-84 and AZP-3601 is PTH 1-34, PTH 1-84 and AZP-3601, respectively.

As used herein the term “PTH” refers to all PTH polypeptides, such as from mammalian species, in particular from human and mammalian species, more particularly from human and murine species, as well as their variants, analogs, orthologs, homologs, and derivatives and fragments thereof, that are characterized by raising serum calcium and renal phosphorus excretion and lowering serum phosphorus and renal calcium excretion. The term “PTH” also refers to all PTHrP polypeptides, such as the polypeptide of SEQ ID NO:121, that bind to and activate the common PTH/PTHrP1 receptor. In certain embodiments the term “PTH” refers to the PTH polypeptide of SEQ ID NO:51 as well as its variants, homologs and derivatives exhibiting essentially the same biological activity, i.e., raising serum calcium and renal phosphorus excretion, and lowering serum phosphorus and renal calcium excretion. In certain embodiments the term “PTH” refers to the PTH polypeptide of SEQ ID NO:51.

In certain embodiments the term “PTH” refers to one of the following polypeptide sequences:

(PTH 1-84)
SEQ ID NO: 1
SVSEIQLMHNLGKHLNSMERVEWLRKKLQDVHNFVALGAPLAPRDAGSQRPRKKE
DNVLVESHEKSLGEADKADVNVLTKAKSQ
(PTH 1-83)
SEQ ID NO: 2
SVSEIQLMHNLGKHLNSMERVEWLRKKLQDVHNFVALGAPLAPRDAGSQRPRKKE
DNVLVESHEKSLGEADKADVNVLTKAKS
(PTH 1-82)
SEQ ID NO: 3
SVSEIQLMHNLGKHLNSMERVEWLRKKLQDVHNFVALGAPLAPRDAGSQRPRKKE
DNVLVESHEKSLGEADKADVNVLTKAK
(PTH 1-81)
SEQ ID NO: 4
SVSEIQLMHNLGKHLNSMERVEWLRKKLQDVHNFVALGAPLAPRDAGSQRPRKKE
DNVLVESHEKSLGEADKADVNVLTKA
(PTH 1-80)
SEQ ID NO: 5
SVSEIQLMHNLGKHLNSMERVEWLRKKLQDVHNFVALGAPLAPRDAGSQRPRKKE
DNVLVESHEKSLGEADKADVNVLTK
(PTH 1-79)
SEQ ID NO: 6
SVSEIQLMHNLGKHLNSMERVEWLRKKLQDVHNFVALGAPLAPRDAGSQRPRKKE
DNVLVESHEKSLGEADKADVNVLT
(PTH 1-78)
SEQ ID NO: 7
SVSEIQLMHNLGKHLNSMERVEWLRKKLQDVHNFVALGAPLAPRDAGSQRPRKKE
DNVLVESHEKSLGEADKADVNVL
(PTH 1-77)
SEQ ID NO: 8
SVSEIQLMHNLGKHLNSMERVEWLRKKLQDVHNFVALGAPLAPRDAGSQRPRKKE
DNVLVESHEKSLGEADKADVNV
(PTH 1-76)
SEQ ID NO: 9
SVSEIQLMHNLGKHLNSMERVEWLRKKLQDVHNFVALGAPLAPRDAGSQRPRKKE
DNVLVESHEKSLGEADKADVN
(PTH 1-75)
SEQ ID NO: 10
SVSEIQLMHNLGKHLNSMERVEWLRKKLQDVHNFVALGAPLAPRDAGSQRPRKKE
DNVLVESHEKSLGEADKADV
(PTH 1-74)
SEQ ID NO: 11
SVSEIQLMHNLGKHLNSMERVEWLRKKLQDVHNFVALGAPLAPRDAGSQRPRKKE
DNVLVESHEKSLGEADKAD
(PTH 1-73)
SEQ ID NO: 12
SVSEIQLMHNLGKHLNSMERVEWLRKKLQDVHNFVALGAPLAPRDAGSQRPRKKE
DNVLVESHEKSLGEADKA
(PTH 1-72)
SEQ ID NO: 13
SVSEIQLMHNLGKHLNSMERVEWLRKKLQDVHNFVALGAPLAPRDAGSQRPRKKE
DNVLVESHEKSLGEADK
(PTH 1-71)
SEQ ID NO: 14
SVSEIQLMHNLGKHLNSMERVEWLRKKLQDVHNFVALGAPLAPRDAGSQRPRKKE
DNVLVESHEKSLGEAD
(PTH 1-70)
SEQ ID NO: 15
SVSEIQLMHNLGKHLNSMERVEWLRKKLQDVHNFVALGAPLAPRDAGSQRPRKKE
DNVLVESHEKSLGEA
(PTH 1-69)
SEQ ID NO: 16
SVSEIQLMHNLGKHLNSMERVEWLRKKLQDVHNFVALGAPLAPRDAGSQRPRKKE
DNVLVESHEKSLGE
(PTH 1-68)
SEQ ID NO: 17
SVSEIQLMHNLGKHLNSMERVEWLRKKLQDVHNFVALGAPLAPRDAGSQRPRKKE
DNVLVESHEKSLG
(PTH 1-67)
SEQ ID NO: 18
SVSEIQLMHNLGKHLNSMERVEWLRKKLQDVHNFVALGAPLAPRDAGSQRPRKKE
DNVLVESHEKSL
(PTH 1-66)
SEQ ID NO: 19
SVSEIQLMHNLGKHLNSMERVEWLRKKLQDVHNFVALGAPLAPRDAGSQRPRKKE
DNVLVESHEKS
(PTH 1-65)
SEQ ID NO: 20
SVSEIQLMHNLGKHLNSMERVEWLRKKLQDVHNFVALGAPLAPRDAGSQRPRKKE
DNVLVESHEK
(PTH 1-64)
SEQ ID NO: 21
SVSEIQLMHNLGKHLNSMERVEWLRKKLQDVHNFVALGAPLAPRDAGSQRPRKKE
DNVLVESHE
(PTH 1-63)
SEQ ID NO: 22
SVSEIQLMHNLGKHLNSMERVEWLRKKLQDVHNFVALGAPLAPRDAGSQRPRKKE
DNVLVESH
(PTH 1-62)
SEQ ID NO: 23
SVSEIQLMHNLGKHLNSMERVEWLRKKLQDVHNFVALGAPLAPRDAGSQRPRKKE
DNVLVES
(PTH 1-61)
SEQ ID NO: 24
SVSEIQLMHNLGKHLNSMERVEWLRKKLQDVHNFVALGAPLAPRDAGSQRPRKKE
DNVLVE
(PTH 1-60)
SEQ ID NO: 25
SVSEIQLMHNLGKHLNSMERVEWLRKKLQDVHNFVALGAPLAPRDAGSQRPRKKE
DNVLV
(PTH 1-59)
SEQ ID NO: 26
SVSEIQLMHNLGKHLNSMERVEWLRKKLQDVHNFVALGAPLAPRDAGSQRPRKKE
DNVL
(PTH 1-58)
SEQ ID NO: 27
SVSEIQLMHNLGKHLNSMERVEWLRKKLQDVHNFVALGAPLAPRDAGSQRPRKKE
DNV
(PTH 1-57)
SEQ ID NO: 28
SVSEIQLMHNLGKHLNSMERVEWLRKKLQDVHNFVALGAPLAPRDAGSQRPRKKE
DN
(PTH 1-56)
SEQ ID NO: 29
SVSEIQLMHNLGKHLNSMERVEWLRKKLQDVHNFVALGAPLAPRDAGSQRPRKKE
D
(PTH 1-55)
SEQ ID NO: 30
SVSEIQLMHNLGKHLNSMERVEWLRKKLQDVHNFVALGAPLAPRDAGSQRPRKKE
(PTH 1-54)
SEQ ID NO: 31
SVSEIQLMHNLGKHLNSMERVEWLRKKLQDVHNFVALGAPLAPRDAGSQRPRKK
(PTH 1-53)
SEQ ID NO: 32
SVSEIQLMHNLGKHLNSMERVEWLRKKLQDVHNFVALGAPLAPRDAGSQRPRK
(PTH 1-52)
SEQ ID NO: 33
SVSEIQLMHNLGKHLNSMERVEWLRKKLQDVHNFVALGAPLAPRDAGSQRPR
(PTH 1-51)
SEQ ID NO: 34
SVSEIQLMHNLGKHLNSMERVEWLRKKLQDVHNFVALGAPLAPRDAGSQRP
(PTH 1-50)
SEQ ID NO: 35
SVSEIQLMHNLGKHLNSMERVEWLRKKLQDVHNFVALGAPLAPRDAGSQR
(PTH 1-49)
SEQ ID NO: 36
SVSEIQLMHNLGKHLNSMERVEWLRKKLQDVHNFVALGAPLAPRDAGSQ
(PTH 1-48)
SEQ ID NO: 37
SVSEIQLMHNLGKHLNSMERVEWLRKKLQDVHNFVALGAPLAPRDAGS
(PTH 1-47)
SEQ ID NO: 38
SVSEIQLMHNLGKHLNSMERVEWLRKKLQDVHNFVALGAPLAPRDAG
(PTH 1-46)
SEQ ID NO: 39
SVSEIQLMHNLGKHLNSMERVEWLRKKLQDVHNFVALGAPLAPRDA
(PTH 1-45)
SEQ ID NO: 40
SVSEIQLMHNLGKHLNSMERVEWLRKKLQDVHNFVALGAPLAPRD
(PTH 1-44)
SEQ ID NO: 41
SVSEIQLMHNLGKHLNSMERVEWLRKKLQDVHNFVALGAPLAPR
(PTH 1-43)
SEQ ID NO: 42
SVSEIQLMHNLGKHLNSMERVEWLRKKLQDVHNFVALGAPLAP
(PTH 1-42)
SEQ ID NO: 43
SVSEIQLMHNLGKHLNSMERVEWLRKKLQDVHNFVALGAPLA
(PTH 1-41)
SEQ ID NO: 44
SVSEIQLMHNLGKHLNSMERVEWLRKKLQDVHNFVALGAPL
(PTH 1-40)
SEQ ID NO: 45
SVSEIQLMHNLGKHLNSMERVEWLRKKLQDVHNFVALGAP
(PTH 1-39)
SEQ ID NO: 46
SVSEIQLMHNLGKHLNSMERVEWLRKKLQDVHNFVALGA
(PTH 1-38)
SEQ ID NO: 47
SVSEIQLMHNLGKHLNSMERVEWLRKKLQDVHNFVALG
(PTH 1-37)
SEQ ID NO: 48
SVSEIQLMHNLGKHLNSMERVEWLRKKLQDVHNFVAL
(PTH 1-36)
SEQ ID NO: 49
SVSEIQLMHNLGKHLNSMERVEWLRKKLQDVHNFVA
(PTH 1-35)
SEQ ID NO: 50
SVSEIQLMHNLGKHLNSMERVEWLRKKLQDVHNFV
(PTH 1-34)
SEQ ID NO: 51
SVSEIQLMHNLGKHLNSMERVEWLRKKLQDVHNF
(PTH 1-33)
SEQ ID NO: 52
SVSEIQLMHNLGKHLNSMERVEWLRKKLQDVHN
(PTH 1-32)
SEQ ID NO: 53
SVSEIQLMHNLGKHLNSMERVEWLRKKLQDVH
(PTH 1-31)
SEQ ID NO: 54
SVSEIQLMHNLGKHLNSMERVEWLRKKLQDV
(PTH 1-30)
SEQ ID NO: 55
SVSEIQLMHNLGKHLNSMERVEWLRKKLQD
(PTH 1-29)
SEQ ID NO: 56
SVSEIQLMHNLGKHLNSMERVEWLRKKLQ
(PTH 1-28)
SEQ ID NO: 57
SVSEIQLMHNLGKHLNSMERVEWLRKKL
(PTH 1-27)
SEQ ID NO: 58
SVSEIQLMHNLGKHLNSMERVEWLRKK
(PTH 1-26)
SEQ ID NO: 59
SVSEIQLMHNLGKHLNSMERVEWLRK
(PTH 1-25)
SEQ ID NO: 60
SVSEIQLMHNLGKHLNSMERVEWLR
(amidated PTH 1-84)
SEQ ID NO: 61
SVSEIQLMHNLGKHLNSMERVEWLRKKLQDVHNFVALGAPLAPRDAGSQRPRKKE
DNVLVESHEKSLGEADKADVNVLTKAKSQ;
wherein the C-terminus is amidated
(amidated PTH 1-83)
SEQ ID NO: 62
SVSEIQLMHNLGKHLNSMERVEWLRKKLQDVHNFVALGAPLAPRDAGSQRPRKKE
DNVLVESHEKSLGEADKADVNVLTKAKS;
wherein the C-terminus is amidated
(amidated PTH 1-82)
SEQ ID NO: 63
SVSEIQLMHNLGKHLNSMERVEWLRKKLQDVHNFVALGAPLAPRDAGSQRPRKKE
DNVLVESHEKSLGEADKADVNVLTKAK;
wherein the C-terminus is amidated
(amidated PTH 1-81)
SEQ ID NO: 64
SVSEIQLMHNLGKHLNSMERVEWLRKKLQDVHNFVALGAPLAPRDAGSQRPRKKE
DNVLVESHEKSLGEADKADVNVLTKA;
wherein the C-terminus is amidated
(amidated PTH 1-80)
SEQ ID NO: 65
SVSEIQLMHNLGKHLNSMERVEWLRKKLQDVHNFVALGAPLAPRDAGSQRPRKKE
DNVLVESHEKSLGEADKADVNVLTK;
wherein the C-terminus is amidated
(amidated PTH 1-79)
SEQ ID NO: 66
SVSEIQLMHNLGKHLNSMERVEWLRKKLQDVHNFVALGAPLAPRDAGSQRPRKKE
DNVLVESHEKSLGEADKADVNVLT;
wherein the C-terminus is amidated
(amidated PTH 1-78)
SEQ ID NO: 67
SVSEIQLMHNLGKHLNSMERVEWLRKKLQDVHNFVALGAPLAPRDAGSQRPRKKE
DNVLVESHEKSLGEADKADVNVL;
wherein the C-terminus is amidated
(amidated PTH 1-77)
SEQ ID NO: 68
SVSEIQLMHNLGKHLNSMERVEWLRKKLQDVHNFVALGAPLAPRDAGSQRPRKKE
DNVLVESHEKSLGEADKADVNV;
wherein the C-terminus is amidated
(amidated PTH 1-76)
SEQ ID NO: 69
SVSEIQLMHNLGKHLNSMERVEWLRKKLQDVHNFVALGAPLAPRDAGSQRPRKKE
DNVLVESHEKSLGEADKADVN;
wherein the C-terminus is amidated
(amidated PTH 1-75)
SEQ ID NO: 70
SVSEIQLMHNLGKHLNSMERVEWLRKKLQDVHNFVALGAPLAPRDAGSQRPRKKE
DNVLVESHEKSLGEADKADV;
wherein the C-terminus is amidated
(amidated PTH 1-74)
SEQ ID NO: 71
SVSEIQLMHNLGKHLNSMERVEWLRKKLQDVHNFVALGAPLAPRDAGSQRPRKKE
DNVLVESHEKSLGEADKAD;
wherein the C-terminus is amidated
(amidated PTH 1-73)
SEQ ID NO: 72
SVSEIQLMHNLGKHLNSMERVEWLRKKLQDVHNFVALGAPLAPRDAGSQRPRKKE
DNVLVESHEKSLGEADKA;
wherein the C-terminus is amidated
(amidated PTH 1-72)
SEQ ID NO: 73
SVSEIQLMHNLGKHLNSMERVEWLRKKLQDVHNFVALGAPLAPRDAGSQRPRKKE
DNVLVESHEKSLGEADK;
wherein the C-terminus is amidated
(amidated PTH 1-71)
SEQ ID NO: 74
SVSEIQLMHNLGKHLNSMERVEWLRKKLQDVHNFVALGAPLAPRDAGSQRPRKKE
DNVLVESHEKSLGEAD;
wherein the C-terminus is amidated
(amidated PTH 1-70)
SEQ ID NO: 75
SVSEIQLMHNLGKHLNSMERVEWLRKKLQDVHNFVALGAPLAPRDAGSQRPRKKE
DNVLVESHEKSLGEA;
wherein the C-terminus is amidated
(amidated PTH 1-69)
SEQ ID NO: 76
SVSEIQLMHNLGKHLNSMERVEWLRKKLQDVHNFVALGAPLAPRDAGSQRPRKKE
DNVLVESHEKSLGE;
wherein the C-terminus is amidated
(amidated PTH 1-68)
SEQ ID NO: 77
SVSEIQLMHNLGKHLNSMERVEWLRKKLQDVHNFVALGAPLAPRDAGSQRPRKKE
DNVLVESHEKSLG;
wherein the C-terminus is amidated
(amidated PTH 1-67)
SEQ ID NO: 78
SVSEIQLMHNLGKHLNSMERVEWLRKKLQDVHNFVALGAPLAPRDAGSQRPRKKE
DNVLVESHEKSL;
wherein the C-terminus is amidated
(amidated PTH 1-66)
SEQ ID NO: 79
SVSEIQLMHNLGKHLNSMERVEWLRKKLQDVHNFVALGAPLAPRDAGSQRPRKKE
DNVLVESHEKS;
wherein the C-terminus is amidated
(amidated PTH 1-65)
SEQ ID NO: 80
SVSEIQLMHNLGKHLNSMERVEWLRKKLQDVHNFVALGAPLAPRDAGSQRPRKKE
DNVLVESHEK;
wherein the C-terminus is amidated
(amidated PTH 1-64)
SEQ ID NO: 81
SVSEIQLMHNLGKHLNSMERVEWLRKKLQDVHNFVALGAPLAPRDAGSQRPRKKE
DNVLVESHE;
wherein the C-terminus is amidated
(amidated PTH 1-63)
SEQ ID NO: 82
SVSEIQLMHNLGKHLNSMERVEWLRKKLQDVHNFVALGAPLAPRDAGSQRPRKKE
DNVLVESH;
wherein the C-terminus is amidated
(amidated PTH 1-62)
SEQ ID NO: 83
SVSEIQLMHNLGKHLNSMERVEWLRKKLQDVHNFVALGAPLAPRDAGSQRPRKKE
DNVLVES;
wherein the C-terminus is amidated
(amidated PTH 1-61)
SEQ ID NO: 84
SVSEIQLMHNLGKHLNSMERVEWLRKKLQDVHNFVALGAPLAPRDAGSQRPRKKE
DNVLVE;
wherein the C-terminus is amidated
(amidated PTH 1-60)
SEQ ID NO: 85
SVSEIQLMHNLGKHLNSMERVEWLRKKLQDVHNFVALGAPLAPRDAGSQRPRKKE
DNVLV;
wherein the C-terminus is amidated
(amidated PTH 1-59)
SEQ ID NO: 86
SVSEIQLMHNLGKHLNSMERVEWLRKKLQDVHNFVALGAPLAPRDAGSQRPRKKE
DNVL;
wherein the C-terminus is amidated
(amidated PTH 1-58)
SEQ ID NO: 87
SVSEIQLMHNLGKHLNSMERVEWLRKKLQDVHNFVALGAPLAPRDAGSQRPRKKE
DNV;
wherein the C-terminus is amidated
(amidated PTH 1-57)
SEQ ID NO: 88
SVSEIQLMHNLGKHLNSMERVEWLRKKLQDVHNFVALGAPLAPRDAGSQRPRKKE
DN;
wherein the C-terminus is amidated
(amidated PTH 1-56)
SEQ ID NO: 89
SVSEIQLMHNLGKHLNSMERVEWLRKKLQDVHNFVALGAPLAPRDAGSQRPRKKE
D;
wherein the C-terminus is amidated
(amidated PTH 1-55)
SEQ ID NO: 90
SVSEIQLMHNLGKHLNSMERVEWLRKKLQDVHNFVALGAPLAPRDAGSQRPRKKE;
wherein the C-terminus is amidated
(amidated PTH 1-54)
SEQ ID NO: 91
SVSEIQLMHNLGKHLNSMERVEWLRKKLQDVHNFVALGAPLAPRDAGSQRPRKK;
wherein the C-terminus is amidated
(amidated PTH 1-53)
SEQ ID NO: 92
SVSEIQLMHNLGKHLNSMERVEWLRKKLQDVHNFVALGAPLAPRDAGSQRPRK;
wherein the C-terminus is amidated
(amidated PTH 1-52)
SEQ ID NO: 93
SVSEIQLMHNLGKHLNSMERVEWLRKKLQDVHNFVALGAPLAPRDAGSQRPR;
wherein the C-terminus is amidated
(amidated PTH 1-51)
SEQ ID NO: 94
SVSEIQLMHNLGKHLNSMERVEWLRKKLQDVHNFVALGAPLAPRDAGSQRP;
wherein the C-terminus is amidated
(amidated PTH 1-50)
SEQ ID NO: 95
SVSEIQLMHNLGKHLNSMERVEWLRKKLQDVHNFVALGAPLAPRDAGSQR;
wherein the C-terminus is amidated
(amidated PTH 1-49)
SEQ ID NO: 96
SVSEIQLMHNLGKHLNSMERVEWLRKKLQDVHNFVALGAPLAPRDAGSQ;
wherein the C-terminus is amidated
(amidated PTH 1-48)
SEQ ID NO: 97
SVSEIQLMHNLGKHLNSMERVEWLRKKLQDVHNFVALGAPLAPRDAGS;
wherein the C-terminus is amidated
(amidated PTH 1-47)
SEQ ID NO: 98
SVSEIQLMHNLGKHLNSMERVEWLRKKLQDVHNFVALGAPLAPRDAG;
wherein the C-terminus is amidated
(amidated PTH 1-46)
SEQ ID NO: 99
SVSEIQLMHNLGKHLNSMERVEWLRKKLQDVHNFVALGAPLAPRDA;
wherein the C-terminus is amidated
(amidated PTH 1-45)
SEQ ID NO: 100
SVSEIQLMHNLGKHLNSMERVEWLRKKLQDVHNFVALGAPLAPRD;
wherein the C-terminus is amidated
(amidated PTH 1-44)
SEQ ID NO: 101
SVSEIQLMHNLGKHLNSMERVEWLRKKLQDVHNFVALGAPLAPR;
wherein the C-terminus is amidated
(amidated PTH 1-43)
SEQ ID NO: 102
SVSEIQLMHNLGKHLNSMERVEWLRKKLQDVHNFVALGAPLAP;
wherein the C-terminus is amidated
(amidated PTH 1-42)
SEQ ID NO: 103
SVSEIQLMHNLGKHLNSMERVEWLRKKLQDVHNFVALGAPLA;
wherein the C-terminus is amidated
(amidated PTH 1-41)
SEQ ID NO: 104
SVSEIQLMHNLGKHLNSMERVEWLRKKLQDVHNFVALGAPL;
wherein the C-terminus is amidated
(amidated PTH 1-40)
SEQ ID NO: 105
SVSEIQLMHNLGKHLNSMERVEWLRKKLQDVHNFVALGAP;
wherein the C-terminus is amidated
(amidated PTH 1-39)
SEQ ID NO: 106
SVSEIQLMHNLGKHLNSMERVEWLRKKLQDVHNFVALGA;
wherein the C-terminus is amidated
(amidated PTH 1-38)
SEQ ID NO: 107
SVSEIQLMHNLGKHLNSMERVEWLRKKLQDVHNFVALG;
wherein the C-terminus is amidated
(amidated PTH 1-37)
SEQ ID NO: 108
SVSEIQLMHNLGKHLNSMERVEWLRKKLQDVHNFVAL;
wherein the C-terminus is amidated
(amidated PTH 1-36)
SEQ ID NO: 109
SVSEIQLMHNLGKHLNSMERVEWLRKKLQDVHNFVA;
wherein the C-terminus is amidated
(amidated PTH 1-35)
SEQ ID NO: 110
SVSEIQLMHNLGKHLNSMERVEWLRKKLQDVHNFV;
wherein the C-terminus is amidated
(amidated PTH 1-34)
SEQ ID NO: 111
SVSEIQLMHNLGKHLNSMERVEWLRKKLQDVHNF;
wherein the C-terminus is amidated
(amidated PTH 1-33)
SEQ ID NO: 112
SVSEIQLMHNLGKHLNSMERVEWLRKKLQDVHN;
wherein the C-terminus is amidated
(amidated PTH 1-32)
SEQ ID NO: 113
SVSEIQLMHNLGKHLNSMERVEWLRKKLQDVH;
wherein the C-terminus is amidated
(amidated PTH 1-31)
SEQ ID NO: 114
SVSEIQLMHNLGKHLNSMERVEWLRKKLQDV;
wherein the C-terminus is amidated
(amidated PTH 1-30)
SEQ ID NO: 115
SVSEIQLMHNLGKHLNSMERVEWLRKKLQD;
wherein the C-terminus is amidated
(amidated PTH 1-29)
SEQ ID NO: 116
SVSEIQLMHNLGKHLNSMERVEWLRKKLQ;
wherein the C-terminus is amidated
(amidated PTH 1-28)
SEQ ID NO: 117
SVSEIQLMHNLGKHLNSMERVEWLRKKL;
wherein the C-terminus is amidated
(amidated PTH 1-27)
SEQ ID NO: 118
SVSEIQLMHNLGKHLNSMERVEWLRKK;
wherein the C-terminus is amidated
(amidated PTH 1-26)
SEQ ID NO: 119
SVSEIQLMHNLGKHLNSMERVEWLRK;
wherein the C-terminus is amidated
(amidated PTH 1-25)
SEQ ID NO: 120
SVSEIQLMHNLGKHLNSMERVEWLR;
wherein the C-terminus is amidated
(PTHrP)
SEQ ID NO: 121
AVSEHQLLHDKGKSIQDLRRRFFLHHLIAEIHTAEIRATSEVSPNSKPSPNTKNHP
VRFGSDDEGRYLTQETNKVETYKEQPLKTPGKKKKGKPGKRKEQEKKKRRTRSAWL
DSGVTGSGLEGDHLSDTSTTSLELDSRRH

and sequences having at least 90%, such as at least 91%, at least 92%, at least 93%, at least 94%, at least 95%, at least 96%, at least 97%, at least 98% or at least 99% homology thereto.

The terms “PTH molecule” and “PTH moiety” also include poly(amino acid) conjugates which have a sequence as described above, but having a backbone that comprises both amide and non-amide linkages, such as ester linkages, like for example depsipeptides. Depsipeptides are chains of amino acid residues in which the backbone comprises both amide (peptide) and ester bonds. Accordingly, the term “side chain” as used herein refers either to the moiety attached to the alpha-carbon of an amino acid moiety, if the amino acid moiety is connected through amine bonds such as in proteins and peptides, or to any carbon atom-comprising moiety attached to the backbone of a poly(amino acid) conjugate, such as for example in the case of depsipeptides.

In certain embodiments the term “PTH” refers to sequences having a backbone formed through amide (peptide) bonds.

As used herein the term “long-acting PTH compound” refers to a compound comprising a PTH molecule or PTH moiety that is capable of maintaining a PD response, such as increasing serum calcium levels, for at least 24 h following administration. As used herein the term “ultra long-acting PTH compound” refers to a compound comprising a PTH molecule or PTH moiety that is capable of maintaining a PD response, such as increasing serum calcium levels, for at least one week following administration.

As used herein the term “sustained-release PTH compound” or “controlled-release PTH compound” refers to any compound, conjugate, crystal or admixture that comprises at least one PTH molecule or PTH moiety and from which the at least one PTH molecule or PTH moiety is released with a release half-life of at least 12 hours.

As used herein the terms “release half-life” and “half-life” refer to the time required under physiological conditions (i.e. aqueous buffer, pH 7.4, 37° C.) until half of all PTH molecules or PTH moieties, respectively, of a sustained-release PTH compound or of a PTH prodrug are released.

The term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties. As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties. For simplification, PTH moieties and PTH molecules are generally referred to herein as “protein”.

As used herein the term “physiological conditions” refers to aqueous buffer at pH 7.4, 37° C.

As used herein the term “pharmaceutical composition” refers to a composition containing one or more active ingredients, such as for example at least one PTH compound, and one or more excipients, as well as any product which results, directly or indirectly, from combination, complexation or aggregation of any two or more of the ingredients of the composition, or from dissociation of one or more of the ingredients, or from other types of reactions or interactions of one or more of the ingredients. Accordingly, a pharmaceutical composition for use of the present invention encompasses any composition made by admixing one or more PTH compound and a pharmaceutically acceptable excipient.

As used herein, the term “excipient” refers to a diluent, adjuvant, or vehicle with which the therapeutic, such as a drug or prodrug, is administered. Such pharmaceutical excipient may be sterile liquids, such as water and oils, including those of petroleum, animal, vegetable or synthetic origin, including but not limited to peanut oil, soybean oil, mineral oil, sesame oil and the like. Water is an example for an excipient when the pharmaceutical composition is administered orally. Saline and aqueous dextrose are examples of excipients when the pharmaceutical composition is administered intravenously. Saline solutions and aqueous dextrose and glycerol solutions are in certain embodiments employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or can contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, or sustained-release formulations. The pharmaceutical composition may be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or biologically active moiety, together with a suitable amount of excipient so as to provide the form for proper administration to the patient. The formulation should suit the mode of administration.

As used herein the term “liquid composition” refers to a mixture comprising a water-soluble PTH compound and one or more solvents, such as water.

The term “suspension composition” relates to a mixture comprising at least one water-insoluble PTH compound and one or more solvents, such as water.

As used herein, the term “dry composition” means that a pharmaceutical composition is provided in a dry form. Suitable methods for drying are spray-drying and lyophilization, i.e., freeze-drying. Such dry composition has a residual water content of a maximum of 10%, such as less than 5% or less than 2%, determined according to Karl Fischer. In certain embodiments such dry pharmaceutical composition is dried by lyophilization.

The term “drug” as used herein refers to a substance, such as PTH, used in the treatment, cure, prevention, or diagnosis of a disease or used to otherwise enhance physical or mental well-being. If a drug is conjugated to another moiety, the moiety of the resulting product that originated from the drug is referred to as “drug moiety”.

As used herein the term “prodrug” refers to a covalent conjugate in which a drug moiety is reversibly and covalently connected to a specialized protective group through a reversible linker moiety, also referred to as “reversible prodrug linker moiety” or “reversible linker moiety”, which is conjugated through a reversible linkage to the biologically active moiety and wherein the specialized protective group alters or eliminates undesirable properties in the parent molecule. This also includes the enhancement of desirable properties in the drug and the suppression of undesirable properties. The specialized non-toxic protective group is referred to as “carrier”. A prodrug releases the reversibly and covalently bound drug moiety in the form of its corresponding drug. In other words, a prodrug is a conjugate comprising a drug moiety which is covalently and reversibly conjugated to a carrier moiety via a reversible linker moiety, which covalent and reversible conjugation of the carrier to the reversible linker moiety is either directly or through a spacer. Such conjugate releases the formerly conjugated drug moiety in the form of a free unmodified drug.

A “reversible linkage” is a linkage that is degradable, i.e. cleavable, in the absence of enzymes under physiological conditions (aqueous buffer at pH 7.4, 37° C.) with a half-life ranging from one hour to three months, in certain embodiments from one hour to two months, in certain embodiments from one hour to one month, in certain embodiments from one hour to three weeks, in certain embodiments from one hour to two weeks, in certain embodiments from 12 hours to two weeks, in certain embodiments from 12 hours to one week. Accordingly, a stable linkage is a linkage having a half-life under physiological conditions (aqueous buffer at pH 7.4, 37° C.) in the absence of enzymes of more than three months.

As used herein, the terms “traceless prodrug linker” or “traceless linker” means a reversible prodrug linker, i.e. a linker moiety reversibly and covalently connecting a drug moiety with a carrier, which upon cleavage releases the drug in its free form. As used herein, the term “free form” of a drug means the drug in its unmodified, pharmacologically active form.

As used herein, the term “reagent” means a chemical compound which comprises at least one functional group for reaction with the functional group of another chemical compound or drug.

It is understood that a drug comprising a functional group, such as a primary or secondary amine or hydroxyl functional group is also a reagent.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a drug moiety is released from a prodrug as a drug.

It is understood that if the chemical structure of a group of atoms is provided which group of atoms is attached to at least one other moiety or is interrupting a moiety, said chemical structure may be attached to the at least one further or interrupted moiety in either orientation, unless explicitly stated otherwise. For example, a moiety “—C(O)N(R′)—” may be attached to two moieties or interrupting a moiety either as “—C(O)N(R¹)—” or as “—N(R¹)C(O)—”. Similarly, a moiety

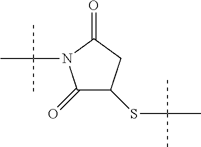

may be attached to two moieties or can interrupt a moiety either as

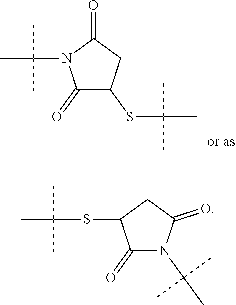

As used herein, the term “functional group” means a group of atoms which can react with other groups of atoms. Functional groups include but are not limited to the following groups: carboxylic acid, primary or secondary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane, and aziridine.

In case a PTH compound comprises one or more acidic or basic groups, the invention also comprises their corresponding pharmaceutically or toxicologically acceptable salts, in particular their pharmaceutically utilizable salts. Thus, the PTH compound comprising acidic groups may be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. A PTH compound comprising one or more basic groups, i.e. groups which can be protonated, may be present and may be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. For the person skilled in the art further methods are known for converting the basic group into a cation like the alkylation of an amine group resulting in a positively-charge ammonium group and an appropriate counterion of the salt. If the PTH compound simultaneously comprises acidic and basic groups, the invention also includes, in addition to the salt forms mentioned above, inner salts or betaines (zwitterions). The respective salts may be obtained by customary methods which are known to the person skilled in the art like, for example by contacting these compounds with an organic or inorganic acid or base in a solvent or dispersant, or by anion exchange or cation exchange with other salts. The present invention also includes all salts of the compounds which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means a substance that does not cause harm when administered to a patient and in certain embodiments means approved by a regulatory agency, such as the EMA (Europe) and/or the FDA (US) and/or any other national regulatory agency for use in animals, in particular for use in humans.

As used herein the terms “about” or “approx.” in combination with a numerical value is used to indicate a range ranging from and including the numerical value plus and minus no more than 10% of said numerical value, in certain embodiments no more than 8% of said numerical value, in certain embodiments no more than 5% of said numerical value and in certain embodiments no more than 2% of said numerical value. For example, the phrases “about 200” or “approx. 200” is used to mean a range ranging from and including 200+/−10%, i.e. ranging from and including 180 to 220; in certain embodiments 200+/−8%, i.e. ranging from and including 184 to 216; in certain embodiments ranging from and including 200+/−5%, i.e. ranging from and including 190 to 210; and in certain embodiments 200+/−2%, i.e. ranging from and including 196 to 204. It is understood that a percentage given as “about 20%” or “approx. 20%” does not mean “20%+/−10%”, i.e. ranging from and including 10 to 30%, but “about 20%” or “approx. 20%” means ranging from and including 18 to 22%, i.e. plus and minus 10% of the numerical value which is 20.

As used herein, the term “polymer” means a molecule comprising repeating structural units, i.e., the monomers, connected by chemical bonds in a linear, circular, branched, crosslinked or dendrimeric way or a combination thereof, which may be of synthetic or biological origin or a combination of both. It is understood that a polymer may also comprise one or more other chemical groups and/or moieties, such as, for example, one or more functional groups. In certain embodiments a soluble polymer has a molecular weight of at least 0.5 kDa, e.g., a molecular weight of at least 1 kDa, a molecular weight of at least 2 kDa, a molecular weight of at least 3 kDa or a molecular weight of at least 5 kDa. If the polymer is soluble, it in certain embodiments has a molecular weight of at most 1000 kDa, such as at most 750 kDa, such as at most 500 kDa, such as at most 300 kDa, such as at most 200 kDa, or such as at most 100 kDa.

It is understood that for water-insoluble polymers, such as hydrogels, no meaningful molecular weight ranges can be provided. It is understood that also a peptide or protein is a polymer in which the amino acids are the repeating structural units, even though the side chains of each amino acid may be different.

As used herein, the term “polymeric” means a reagent or a moiety comprising one or more polymers or polymer moieties. A polymeric reagent or moiety may optionally also comprise one or more other moiety/moieties, which are in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

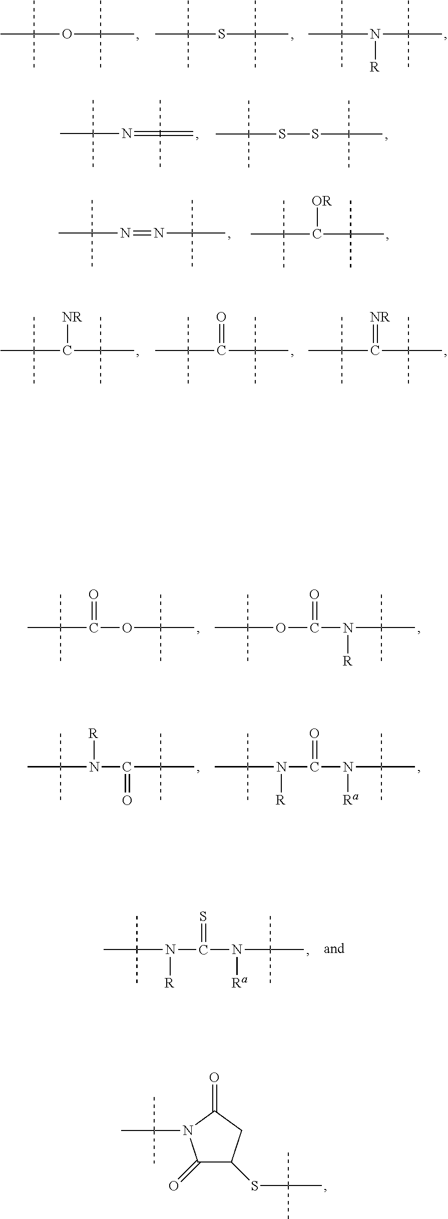

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e., to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lie. An integer for “x” given as “about x” means that the arithmetic mean numbers of monomers lie in a range of integers of x+/−10%, in certain embodiments x+/−8%, in certain embodiments x+/−5% and in certain embodiments x+/−2%.

As used herein, the term “number average molecular weight” means the ordinary arithmetic mean of the molecular weights of the individual polymers.

As used herein the term “water-soluble” with reference to the PTH compound means that at least 1 g of the PTH compound may be dissolved in one liter of water at 20° C. to form a homogeneous solution. Accordingly, the term “water-insoluble” with reference to PTH compound means that less than 1 g of the PTH compound may be dissolved in one liter of water at 20° C. to form a homogeneous solution.

As used herein, the term “PEG-based” in relation to a moiety or reagent means that said moiety or reagent comprises PEG. In certain embodiments a PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60 (w/w) PEG, such as at least 70% (w/w) PEG, such as at least 80% (w/w) PEG, such as at least 90% (w/w) PEG, such as at least 95%. The remaining weight percentage of the PEG-based moiety or reagent are other moieties that in certain embodiments are selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

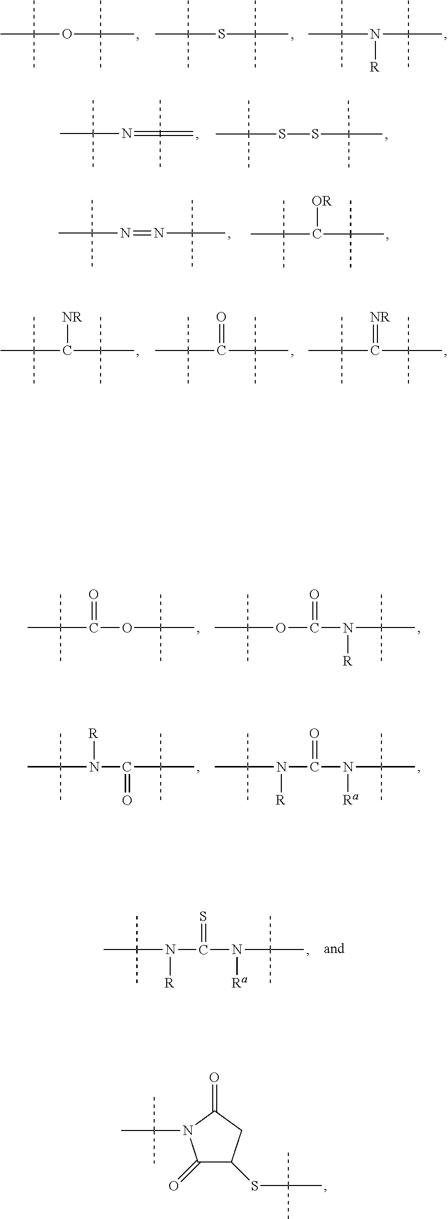

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The term “substituted” as used herein means that one or more —H atom(s) of a molecule or moiety are replaced by a different atom or a group of atoms, which are referred to as “substituent”.

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
—R^{x1}, R^{x1a}, R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4}b), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a}), and —OC(O)N(R^{x3})—;
each —R^{x1}, —R^{x1a}, R^{x1b}, R^{x3}, R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4}b), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4}a —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x4}, —R^{x4a}, —R^{x4b} is independently selected from the group consisting of —H, halogen, C₁₋₆ alkyl, C₂₋₆ alkenyl, and C₂₋₆ alkynyl;

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R², which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{x1b}, —R^{x2}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x}2 which are the same or different.

In certain embodiments a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

The term “interrupted” means that a moiety is inserted between two carbon atoms or—if the insertion is at one of the moiety's ends—between a carbon or heteroatom and a hydrogen atom, in certain embodiments between a carbon and a hydrogen atom.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁0.6 alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀ or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the term “C₂₋₁₀ alkenyl”, “C₂₋₂₀ alkenyl” or “C₂₋₅₀ alkenyl” alone or in combination means a straight-chain or branched hydrocarbon moiety comprising at least one carbon-carbon double bond having 2 to 10, 2 to 20 or 2 to 50 carbon atoms. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C—C≡CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which in certain embodiments are selected from the group consisting of

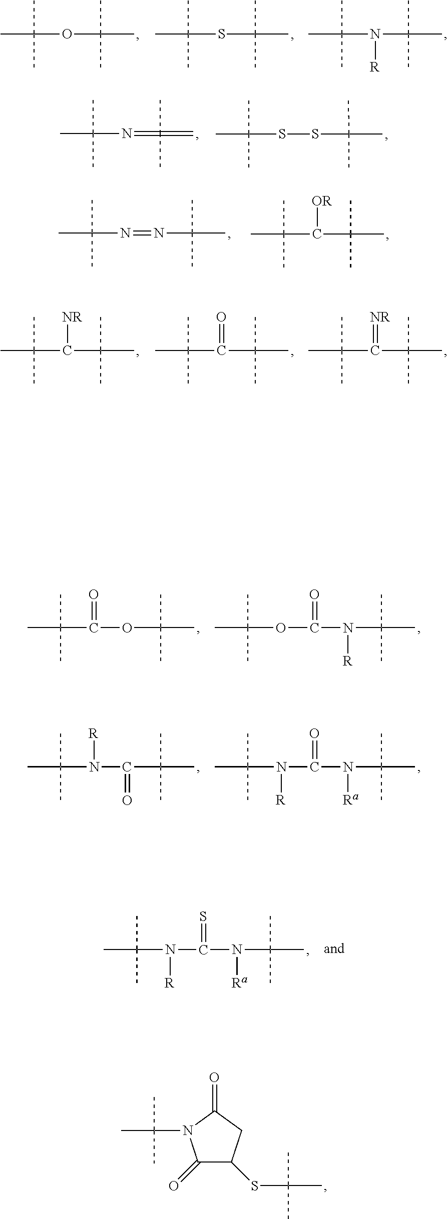

wherein
dashed lines indicate attachment to the remainder of the moiety or reagent; and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

The term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments an 8- to 30-membered carbopolycyclyl means a cyclic moiety of two, three, four or five rings, in certain embodiments of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similary, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)— and —S(O)₂—), oxygen and nitrogen (including ═N(O)—) and wherein the ring is linked to the rest of a molecule via a carbon or nitrogen atom.

It is understood that the phrase “the pair R^{x}/R^{Y} is joined together with the atom to which they are attached to form a C₃0.10 cycloalkyl or a 3- to 10-membered heterocyclyl” in relation with a moiety of the structure

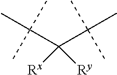

means that R^{x} and R^{y} form the following structure:

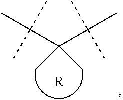

wherein R is C₃₋₁₀ cycloalkyl or 3- to 10-membered heterocyclyl.

It is also understood that the phrase “the pair R^{x}/R^{y} is joint together with the atoms to which they are attached to form a ring A” in relation with a moiety of the structure

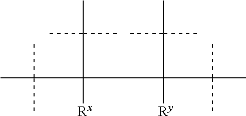

means that R^{x} and R^{y} form the following structure:

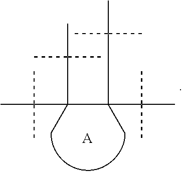

As used herein, “halogen” means fluoro, chloro, bromo or iodo. In certain embodiments halogen is fluoro or chloro.

In general, the term “comprise” or “comprising” also encompasses “consist of” or “consisting of”.

The patient of the first, second and third aspect is a stable patient. Such stable patient may be treated with a first PTH compound prior to initiation of the treatment with the weekly PTH compound, such as a first PTH compound that is administered once every 8 hours, once every 12 hours or once daily, and which first PTH compound is different from the PTH compound administered weekly, which may also be referred to as “weekly PTH compound”. Specific embodiments for the first, second and third aspect are as described elsewhere herein. Specific embodiments for the weekly PTH compound are as described for the second PTH compound.

Unless otherwise stated, the following sections apply to all aspects of the present invention.

In certain embodiments the chronic hypoparathyroidism is due to surgery, such as thyroid or parathyroid gland surgery, a genetic cause, immune system-related damage of the parathyroid glands or is idiopathic. In certain embodiments the chronic hypoparathyroidism is due to surgery. In certain embodiments the chronic hypoparathyroidism is due to thyroid surgery. In certain embodiments the chronic hypoparathyroidism is due to parathyroid gland surgery. In certain embodiments the chronic hypoparathyroidism is due to a genetic cause. In certain embodiments the chronic hypoparathyroidism is due to autosomal dominant hypocalcemia type I. In certain embodiments the chronic hypoparathyroidism is due to immune-related damage of the parathyroid glands. In certain embodiments the chronic hypoparathyroidism is idiopathic.

In certain embodiments the patient is a mammalian patient. In certain embodiments the patient is a human patient, such as an adult or pediatric patient. In certain embodiments the patient has chronic hypoparathyroidism due to surgery, a genetic cause, immune system-related damage of the parathyroid glands or the hypoparathyroidism is idiopathic. In certain embodiments the patient has chronic hypoparathyroidism due to surgery. In certain embodiments the patient has chronic hypoparathyroidism due to thyroid surgery. In certain embodiments the patient has chronic hypoparathyroidism due to parathyroid gland surgery. In certain embodiments the patient has chronic hypoparathyroidism due to a genetic cause. In certain embodiments the patient has chronic hypoparathyroidism due to autosomal dominant hypocalcemia type I. In certain embodiments the patient has chronic hypoparathyroidism due to immune-related damage of the parathyroid glands. In certain embodiments the patient has idiopathic chronic hypoparathyroidism.

In certain embodiments the patient is an adult patient having chronic hypoparathyroidism due to surgery, a genetic cause, immune system-related damage of the parathyroid glands or the hypoparathyroidism is idiopathic. In certain embodiments the patient is an adult human patient having chronic hypoparathyroidism due to surgery. In certain embodiments the patient is an adult human patient having chronic hypoparathyroidism due to thyroid surgery. In certain embodiments the patient is an adult human patient having chronic hypoparathyroidism due to parathyroid gland surgery. In certain embodiments the patient is an adult human patient having chronic hypoparathyroidism due to a genetic cause. In certain embodiments the patient is an adult human patient having chronic hypoparathyroidism due to autosomal dominant hypocalcemia type I. In certain embodiments the patient is an adult human patient having chronic hypoparathyroidism due to immune-related damage of the parathyroid glands. In certain embodiments the patient is an adult human patient having idiopathic chronic hypoparathyroidism.

In certain embodiments the patient is a pediatric patient having chronic hypoparathyroidism due to surgery, a genetic cause, immune system-related damage of the parathyroid glands or the hypoparathyroidism is idiopathic. In certain embodiments the patient is a pediatric human patient having chronic hypoparathyroidism due to surgery. In certain embodiments the patient is a pediatric human patient having chronic hypoparathyroidism due to thyroid surgery. In certain embodiments the patient is a pediatric human patient having chronic hypoparathyroidism due to parathyroid gland surgery. In certain embodiments the patient is a pediatric human patient having chronic hypoparathyroidism due to a genetic cause. In certain embodiments the patient is a pediatric human patient having chronic hypoparathyroidism due to autosomal dominant hypocalcemia type I. In certain embodiments the patient is a pediatric human patient having chronic hypoparathyroidism due to immune-related damage of the parathyroid glands. In certain embodiments the patient is a pediatric human patient having idiopathic chronic hypoparathyroidism.

In certain embodiments the patient is controlled on conventional therapy, i.e., active vitamin D and calcium supplement, prior to starting treatment with the first PTH compound.

In certain embodiments the patient receives active vitamin D and calcium supplement before step (a).

Upon initiation of the treatment with the first PTH compound the dose of active vitamin D and calcium supplement administered to the patient is reduced, until administration of active vitamin D and calcium is completely eliminated. This reduction or elimination can be accomplished by one or more adjustments of the dose of active vitamin D and/or calcium supplement. During the time the patient is titrated off of active vitamin D and calcium supplement the dose of the first PTH compound may be increased or decreased on one or more occasions. Such increase or decrease of the dose of the first PTH compound may be made in response to hypo- or hypercalcemia, respectively.

In certain embodiments the first PTH compound may be administered to a patient by various modes of administration, such as via topical, enteral or parenteral administration or by methods of external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection and infusion, direct delivery to the brain via implanted device allowing delivery of the invention or the like to brain tissue or brain fluids (e.g., Ommaya Reservoir), direct intracerebroventricular injection or infusion, injection or infusion into brain or brain associated regions, injection into the subchoroidal space, retro-orbital injection and ocular instillation. In certain embodiments the first PTH compound is administered by subcutaneous, intramuscular or oral administration.

In certain embodiments the first PTH compound is administered by subcutaneous administration, such as by subcutaneous injection.

In certain embodiments the first PTH compound is administered using a pen injector. In certain embodiments the first PTH compound is administered using a needle and syringe. In certain embodiments the first PTH compound is administered via subcutaneous injection using a pen injector. In certain embodiments the first PTH compound is administered via subcutaneous injection using a needle and syringe.

In certain embodiments the first PTH compound is the only drug comprising PTH or a PTH moiety administered prior to administration of the weekly administered PTH compound of the first, second or third embodiment. Such first PTH compound is administered with a first average interval between two consecutive administrations.

In certain embodiments the first PTH compound is the only drug comprising PTH or a PTH moiety administered to the patient during step (a).

In certain embodiments the first average interval ranges from 6 hours to one week. In certain embodiments the first average interval ranges from 6 hours to one day. In certain embodiments the first average interval is approx. 8 hours. In certain embodiments the first average interval is 8 hours. In certain embodiments the first average interval is approx. 12 hours. In certain embodiments the first average interval is 12 hours. In certain embodiments the first average interval is approx. one day. In certain embodiments the first average interval is one day. In certain embodiments the first average interval is approx. two days. In certain embodiments the first average interval is two days. In certain embodiments the first average interval is approx. three days. In certain embodiments the first average interval is three days. In certain embodiments the first average interval is approx. four days. In certain embodiments the first average interval is four days. In certain embodiments the first average interval is approx. five days. In certain embodiments the first average interval is five days. In certain embodiments the first average interval is approx. six days. In certain embodiments the first average interval is six days. In certain embodiments the first average interval is approx. one week. In certain embodiments the first average interval is one week.

In certain embodiments the intervals between administrations in step (a) are of varying length.

In certain embodiments all intervals between administrations in step (a) have the same length. If all intervals between administrations have the same length, the length of such interval is referred to as “first interval between administration” or short “first interval”.

In certain embodiments the first interval ranges from 6 hours to one week. In certain embodiments the first interval ranges from 6 hours to one day. In certain embodiments the first interval is approx. 8 hours. In certain embodiments the first interval is 8 hours. In certain embodiments the first interval is approx. 12 hours. In certain embodiments the first interval is 12 hours. In certain embodiments the first interval is approx. one day. In certain embodiments the first interval is one day. In certain embodiments the first interval is approx. two days. In certain embodiments the first interval is two days. In certain embodiments the first interval is approx. three days. In certain embodiments the first interval is three days. In certain embodiments the first interval is approx. four days. In certain embodiments the first interval is four days. In certain embodiments the first interval is approx. five days. In certain embodiments the first interval is five days. In certain embodiments the first interval is approx. six days. In certain embodiments the first interval is six days. In certain embodiments the first interval is approx. one week. In certain embodiments the first interval is one week.

The first PTH compound is administered to the patient on multiple occasions in step (a) and the period of time from the first to the last occasion of administration of the first PTH compound in step (a) is referred to as “first treatment period”. In certain embodiments such first treatment period lasts for a period ranging from one week to 20 years. In certain embodiments the first treatment period lasts for a period ranging from one week to 10 years.

In certain embodiments the first treatment period lasts for a period ranging from two weeks to 5 years. In certain embodiments the first treatment period lasts for a period ranging from two weeks to 2 years. In certain embodiments the first treatment period lasts for a period ranging from two weeks to one year. In certain embodiments the first treatment period lasts for a period ranging from two weeks to 6 months. In certain embodiments the first treatment period lasts for a period ranging from two weeks to 4 months. In certain embodiments such first treatment period lasts for at least one week, such as for one week, two weeks, three weeks, four weeks, five weeks, six weeks, seven weeks, eight weeks, nine weeks, ten weeks, eleven weeks, twelve weeks, four months, five months, six months, seven months, eight months, nine months, ten months, eleven months, one year, two years, three years, four years, five years, six years, seven years, eight years, nine years or ten years. It is understood that a patient may be treated with the first PTH compound and be a stable patient but may not initiate treatment with the second PTH compound for some time, which may for example be due to a lack of availability or reimbursement of a suitable second PTH compound in the patient's territory. In certain embodiments the first treatment period lasts at least until the patient has serum calcium levels that are within the normal range and has discontinued conventional therapy, i.e., active vitamin D and calcium supplement.

In certain embodiments the patient has been a stable patient with no adjustments of the dose of the first PTH compound for at least the length of the interval between of the second-last and last administration of the first PTH compound before administration of the first dose of the second PTH compound, i.e., no dose adjustments occurred for at least the last interval of the first treatment period. In certain embodiments the patient has been a stable patient with no dose adjustments for the last two, three, four, five, six, seven, eight, nine, ten, eleven, twelve, thirteen or fourteen average intervals between administrations of the first treatment period before administration of the first dose of the second PTH compound. In certain embodiments the patient has been a stable patient with no dose adjustments for the last two, three, four, five, six, seven, eight, nine, ten, eleven, twelve, thirteen or fourteen intervals of the first treatment period before administration of the first dose of the second PTH compound. In certain embodiments the patient has been a stable patient for at least a week with no dose adjustment before administration of the first dose of the second PTH compound. In certain embodiments the patient has been a stable patient with no dose adjustment for at least a month before administration of the first dose of the second PTH compound. In certain embodiments the patient has been a stable patient with no dose adjustment for at least a year before administration of the first dose of the second PTH compound.

In certain embodiments the second average intervals of the seventh to tenth aspects are of varying length. In certain embodiments the second average intervals of the seventh to tenth aspect have the same length. If all intervals between administrations have the same length, the length of such interval is referred to as “second interval between administrations” or short “second interval”. It is understood that the second interval of the first to the sixth aspect is one week.

In certain embodiments the second interval ranges from one week to two months. In certain embodiments the second interval ranges from one week to one month. In certain embodiments the second interval is about one week. In certain embodiments the second interval is one week.

In certain embodiments the second interval is two weeks.

The second PTH compound is administered to the patient on multiple occasions and the period of time from the first to the last occasion of administration of the second PTH compound is referred to as “second treatment period”. In certain embodiments such second treatment period ranges from one week to 50 years or until the patient dies or until the patient is no longer in need of the second PTH compound. In certain embodiments the patient may switch to a different PTH compound or other form of treatment after the second treatment period.

In certain embodiments the first and/or second PTH compound is/are independently a PTH or parathyroid hormone-related protein (PTHrP) molecule or a pharmaceutically acceptable salt thereof comprising a sequence with at least 90% homology, such as with at least 91% homology, at least 92% homology, at least 93% homology, at least 94% homology, at least 95% homology, at least 96% homology, at least 97% homology, at least 98% homology or at least 99% homology, to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121 and SEQ ID NO:122.

In certain embodiments the first and/or second PTH compound is/are independently a PTH or parathyroid hormone-related protein (PTHrP) molecule or a pharmaceutically acceptable salt thereof comprising a sequence with at least 95% homology to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121 and SEQ ID NO:122.

In certain embodiments the first and/or second PTH compound is/are independently a PTH or PTHrP molecule or a pharmaceutically acceptable salt thereof comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO: 117, SEQ ID NO:118, SEQ ID NO:119, SEQ ID NO:120, SEQ ID NO:121 and SEQ ID NO: 122.

In certain embodiments the first and/or second PTH compound is/are independently a fusion protein comprising at least one PTH or PTHrP molecule or a pharmaceutically acceptable salt thereof comprising a sequence with at least 90% homology, such as with at least 91% homology, at least 92% homology, at least 93% homology, at least 94% homology, at least 95% homology, at least 96% homology, at least 97% homology, at least 98% homology or at least 99% homology, to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO: 117, SEQ ID NO:118, SEQ ID NO:119, SEQ ID NO:120, SEQ ID NO:121 and SEQ ID NO: 122.

In certain embodiments the first and/or second PTH compound is/are independently a fusion protein comprising at least one PTH or PTHrP molecule or a pharmaceutically acceptable salt thereof comprising a sequence with at least 95% homology to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO: 117, SEQ ID NO:118, SEQ ID NO:119, SEQ ID NO:120, SEQ ID NO:121 and SEQ ID NO: 122.

In certain embodiments the first and/or second PTH compound is/are independently a fusion protein comprising at least one PTH or PTHrP molecule or a pharmaceutically acceptable salt thereof comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121 and SEQ ID NO:122.

In certain embodiments the first and/or second PTH compound is/are independently a conjugate comprising a PTH moiety covalently conjugated to one or more further moieties, which may be a polymeric moiety or a fatty acid moiety. In certain embodiments the linkage between the PTH moiety and one further moiety may be reversible. In certain embodiments the linkage between the PTH moiety and one further moiety may be stable. In certain embodiments such PTH compound has one reversible linkage between the PTH moiety and a first further moiety and has a stable linkage between the PTH moiety and a second further moiety.

In certain embodiments such further moiety is a polymeric moiety comprising one or more polymers selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof. In certain embodiments such further moiety is a PEG-based or hyaluronic acid-based moiety. In certain embodiments such further moiety is a PEG-based moiety. In certain embodiments such further moiety is or hyaluronic acid-based moiety.

In certain embodiments such further moiety is a fatty acid moiety, such as the fatty acid moieties disclosed in WO 2005/027978 A2 and WO 2014/060512 A1, which are herewith incorporated by reference.

In certain embodiments the first and/or second PTH compound is/are independently a compound of formula (Ia) or (Ib) or a pharmaceutically acceptable salt thereof

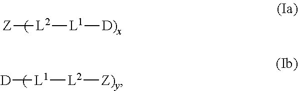

wherein
each -D is independently a PTH moiety;
each -L¹- is independently a linker moiety covalently and reversibly connected to -D;
each -L²- is independently a single chemical bond or a spacer moiety;
each —Z is independently a carrier moiety, such as a fatty acid derivative or a polymer;
x is an integer selected from the group consisting of 1, 2, 3, 4, 5, 6, 7, 8, 9, 10, 11, 12, 13, 14, 15, 16, 17, 18, 19, 20, 21, 22, 23, 24 and 25; and
y is an integer selected from the group consisting of 2, 3, 4 and 5.

The compounds of formula (Ia) and (Ib) may also be referred to as “prodrugs”. They release unmodified PTH in the form of D-H.

In certain embodiments x of formula (Ia) is an integer ranging from 1 to 8. In certain embodiments x of formula (Ia) is an integer ranging from 1 to 6. In certain embodiments x of formula (Ia) is an integer ranging from 1 to 4. In certain embodiments x of formula (Ia) is 1. In certain embodiments x of formula (Ia) is 2. In certain embodiments x of formula (Ia) is 3. In certain embodiments x of formula (Ia) is 4. In certain embodiments x of formula (Ia) is 5. In certain embodiments x of formula (Ia) is 6. In certain embodiments x of formula (Ia) is 7. In certain embodiments x of formula (Ia) is 8. In certain embodiments x of formula (Ia) is 9. In certain embodiments x of formula (Ia) is 10. In certain embodiments x of formula (Ia) is 11. In certain embodiments x of formula (Ia) is 12. In certain embodiments x of formula (Ia) is 13. In certain embodiments x of formula (Ia) is 14. In certain embodiments x of formula (Ia) is 15. In certain embodiments x of formula (Ia) is 16.

In certain embodiments y of formula (Ib) is 2. In certain embodiments y of formula (Ib) is 3.

In certain embodiments y of formula (Ib) is 4. In certain embodiments y of formula (Ib) is 5.

In certain embodiments y of formula (Ib) is 6. In certain embodiments y of formula (Ib) is 7.

In certain embodiments y of formula (Ib) is 8. In certain embodiments y of formula (Ib) is 9.

In certain embodiments y of formula (Ib) is 10. In certain embodiments y of formula (Ib) is 11.

In certain embodiments y of formula (Ib) is 12. In certain embodiments y of formula (Ib) is 13.

In certain embodiments y of formula (Ib) is 14. In certain embodiments y of formula (Ib) is 15.

In certain embodiments y of formula (Ib) is 16. In certain embodiments y of formula (Ib) is 17.

In certain embodiments y of formula (Ib) is 18. In certain embodiments y of formula (Ib) is 19 In certain embodiments y of formula (Ib) is 20.

In certain embodiments the first PTH compound and/or the second PTH compound is/are independently a compound of formula (Ia). In certain embodiments the first PTH compound and/or the second PTH compound is/are independently a compound of formula (Ia) with x=1.

Specific embodiments for -D, -L¹-, -L²- and Z are as described elsewhere herein.

In certain embodiments the first and/or second PTH compound is/are independently a water-insoluble compound, which in certain embodiments is selected from the group of crystals, nanoparticles, microparticles, nanospheres and microspheres. In certain embodiments the first and/or second PTH compound may independently be a crystal comprising at least one PTH molecule. In certain embodiments the first and/or second PTH compound may independently be a nanoparticle comprising at least one PTH molecule. In certain embodiments the first and/or second PTH compound may independently be a microparticle comprising at least one PTH molecule. In certain embodiments the first and/or second PTH compound may independently be a nanosphere comprising at least one PTH compound. In certain embodiments the first and/or second PTH compound may independently be a microsphere comprising at least one PTH compound. In certain embodiments the first and/or second PTH compound may independently be vesicle comprising at least one PTH compound, such as a micelle, liposome or polymersome.

In certain embodiments the first and/or second PTH compound is/are a water-insoluble PTH compound comprising at least one PTH molecule non-covalently embedded in a water-insoluble polymer. In certain embodiments such water-insoluble polymer comprises a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof. In certain embodiments the water-insoluble polymer is poly(lactic-co-glycolic acid) (PLGA).

In certain embodiments the first PTH compound and/or the second PTH compound is/are independently of each other a conjugate or its pharmaceutically acceptable salt comprising a carrier moiety Z′ to which one or more moieties -L²-L¹-D are conjugated, wherein each -L²- is independently a chemical bond or a spacer moiety; each -L¹- is independently a linker moiety to which -D is reversibly and covalently conjugated; each -D is independently a PTH moiety; and Z′ is a hydrogel. Such long-acting PTH compound is a sustained-release PTH compound. Specific embodiments for -D, -L¹-, -L²- and Z′ are as described elsewhere herein.

In certain embodiments -D is a PTH moiety comprising a sequence with at least 90% homology, such as with at least 91% homology, at least 92% homology, at least 93% homology, at least 94% homology, at least 95% homology, at least 96% homology, at least 97% homology, at least 98% homology or at least 99% homology, to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO: 117, SEQ ID NO:118, SEQ ID NO:119, SEQ ID NO:120, SEQ ID NO:121 and SEQ ID NO: 122.

In certain embodiments -D is a PTH moiety comprising a sequence with at least 95% homology to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO: 117, SEQ ID NO:118, SEQ ID NO:119, SEQ ID NO:120, SEQ ID NO:121 and SEQ ID NO: 122.

In certain embodiments -D is a PTH moiety comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121 and SEQ ID NO:122.

In certain embodiments -D is a PTH moiety comprising a sequence with at least 90% homology, such as with at least 91% homology, at least 92% homology, at least 93% homology, at least 94% homology, at least 95% homology, at least 96% homology, at least 97% homology, at least 98% homology or at least 99% homology, to a sequence selected from the group consisting of SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120 and SEQ ID NO: 122.

In certain embodiments -D is a PTH moiety comprising a sequence with at 95% homology to a sequence selected from the group consisting of SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120 and SEQ ID NO:122.

In certain embodiments -D is selected from the group consisting of SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO: 116, SEQ ID NO:117, SEQ ID NO:118, SEQ ID NO:119, SEQ ID NO:120 and SEQ ID NO: 122.

In certain embodiments -D is a PTH moiety comprising a sequence with at least 90% homology, such as with at least 91% homology, at least 92% homology, at least 93% homology, at least 94% homology, at least 95% homology, at least 96% homology, at least 97% homology, at least 98% homology or at least 99% homology, to a sequence selected from the group consisting of SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:107, SEQ ID NO:108, SEQ ID NO:109, SEQ ID NO:110, SEQ ID NO:111, SEQ ID NO:112, SEQ ID NO:113, SEQ ID NO:114 and SEQ ID NO:115.

In certain embodiments -D is a PTH moiety comprising a sequence with at 95% homology to a sequence selected from the group consisting SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:107, SEQ ID NO:108, SEQ ID NO:109, SEQ ID NO:110, SEQ ID NO:111, SEQ ID NO:112, SEQ ID NO:113, SEQ ID NO:114 and SEQ ID NO:115.

In certain embodiments -D is a PTH moiety comprising a sequence selected from the group consisting of SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:107, SEQ ID NO:108, SEQ ID NO:109, SEQ ID NO:110, SEQ ID NO:111, SEQ ID NO:112, SEQ ID NO:113, SEQ ID NO:114 and SEQ ID NO:115.

In certain embodiments -D is a PTH moiety comprising a sequence with at least 90% homology, such as with at least 91% homology, at least 92% homology, at least 93% homology, at least 94% homology, at least 95% homology, at least 96% homology, at least 97% homology, at least 98% homology or at least 99% homology, to a sequence selected from the group consisting of SEQ ID NO:50, SEQ ID NO:51, SEQ ID NO:52, SEQ ID NO:110, SEQ ID NO:111 and SEQ ID NO:112.

In certain embodiments -D is a PTH moiety comprising a sequence with at 95% homology to a sequence selected from the group consisting of SEQ ID NO:50, SEQ ID NO:51, SEQ ID NO:52, SEQ ID NO:110, SEQ ID NO:111 and SEQ ID NO:112 In certain embodiments -D is selected from the group consisting of SEQ ID NO:50, SEQ ID NO:51, SEQ ID NO:52, SEQ ID NO:110, SEQ ID NO:111 and SEQ ID NO:112.

In certain embodiments -D is of SEQ ID NO:50. In certain embodiments -D is of SEQ ID NO:52. In certain embodiments -D is of SEQ ID NO:110. In certain embodiments -D is of SEQ ID NO:111. In certain embodiments -D is of SEQ ID NO: 112.

In certain embodiments -D is of SEQ ID NO:51.

In certain embodiments -D is of SEQ ID NO:122: AVAEIQLMHQRAKWIQDARR RAFLHKLIAEIHTAEI

A moiety -L¹- is either conjugated to a functional group of the side chain of an amino acid residue of -D, to the N-terminal amine functional group or to the C-terminal carboxyl functional group of -D or to a nitrogen atom in the backbone polypeptide chain of -D. Attachment to either the N-terminus or C-terminus can either be directly through the corresponding amine or carboxyl functional group, respectively, or indirectly, wherein a spacer moiety is first conjugated to the amine or carboxyl functional group to which spacer moiety -L¹- is conjugated.

In certain embodiments the amino acid residue of -D to which -L¹- is conjugated comprises a functional group selected from the group consisting carboxylic acid, primary amine, secondary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, sulfate, disulfide, vinyl sulfone, vinyl ketone, diazoalkane, oxirane, guanidine and aziridine. In certain embodiments the amino acid residue of -D to which -L¹- is conjugated comprises a functional group selected from the group consisting of hydroxyl, primary amine, secondary amine and guanidine. In certain embodiments the amino acid residue of -D to which -L¹- is conjugated comprises a primary or secondary amine functional group. In certain embodiments the amino acid residue of -D to which -L¹- is conjugated comprises a primary amine functional group.

If the moiety -L¹- is conjugated to a functional group of the side chain of an amino acid residue of -D, said amino acid residue is selected from the group consisting of proteinogenic amino acid residues and non-proteinogenic amino acid residues. In certain embodiments -L¹- is conjugated to a functional group of the side chain of a proteinogenic amino acid residue of -D. In certain embodiments -L¹- is conjugated to a functional group of the side chain of a non-proteinogenic amino acid residue of -D.

In certain embodiments -L¹- is conjugated to a functional group of the side chain of a proteinogenic amino acid residue of -D. In certain embodiments said proteinogenic amino acid is selected from the group consisting of histidine, lysine, tryptophan, serine, threonine, tyrosine, aspartic acid, glutamic acid and arginine. In certain embodiments said proteinogenic amino acid is selected from the group consisting of lysine, aspartic acid, arginine and serine. In certain embodiments said proteinogenic amino acid is selected from the group consisting of lysine, arginine and serine. In certain embodiments -L¹- is conjugated to a functional group of the side chain of a histidine of -D. In certain embodiments -L¹- is conjugated to a functional group of the side chain of a lysine of -D. In certain embodiments -L¹- is conjugated to a functional group of the side chain of a tryptophan of -D. In certain embodiments -L¹- is conjugated to a functional group of the side chain of a serine of -D. In certain embodiments -L¹- is conjugated to a functional group of the side chain of a threonine of -D. In certain embodiments -L¹- is conjugated to a functional group of the side chain of a tyrosine of -D. In certain embodiments -L¹- is conjugated to a functional group of the side chain of an aspartic acid of -D. In certain embodiments -L¹- is conjugated to a functional group of the side chain of a glutamic acid of -D. In certain embodiments -L¹- is conjugated to a functional group of the side chain of an arginine of -D. It is understood that not every -D moiety may comprise all of these amino acid residues.

In certain embodiments -L¹- is conjugated to the N-terminal amine functional group of -D, either directly through the corresponding amine functional group or indirectly wherein a spacer moiety is first conjugated to the amine functional group to which spacer moiety -L¹- is conjugated. In certain embodiments -L¹- is directly conjugated to the N-terminal amine functional group of -D. In certain embodiments -L¹- is conjugated to the C-terminal functional group of -D, either directly through the corresponding carboxyl functional group or indirectly wherein a spacer moiety is first conjugated to the carboxyl functional group to which spacer moiety -L¹- is conjugated. In certain embodiments -L¹- is directly conjugated to the N-terminal amine functional group of -D.

The moiety -L¹- can be connected to -D through any type of linkage, provided that it is reversible. In certain embodiments -L¹- is connected to -D through a linkage selected from the group consisting of amide, ester, carbamate, acetal, aminal, imine, oxime, hydrazone, disulfide and acylguanidine. In certain embodiments -L¹- is connected to -D through a linkage selected from the group consisting of amide, ester, carbamate and acylguanidin. It is understood that some of these linkages are not reversible per se, but that in the present invention neighboring groups comprised in -L¹- render these linkages reversible. In certain embodiments -L¹- is connected to -D through an ester linkage. In certain embodiments -L¹- is connected to -D through a carbamate linkage. In certain embodiments -L¹- is connected to -D through an acylguanidine. In certain embodiments -L¹- is connected to -D through an amide linkage.

The moiety -L¹- is a reversible prodrug linker from which the drug, i.e. PTH, is released in its free form, i.e. it is a traceless prodrug linker. Suitable prodrug linkers are known in the art, such as for example the reversible prodrug linker moieties disclosed in WO 2005/099768 A2, WO 2006/136586 A2, WO 2011/089216 A1 and WO 2013/024053 A1, which are incorporated by reference herewith.

In certain embodiments -L¹- is a reversible prodrug linker as described in WO 2011/012722 A1, WO 2011/089214 A1, WO 2011/089215 A1, WO 2013/024052 A1 and WO 2013/160340 A1 which are incorporated by reference herewith.

In certain embodiments -L¹- is disclosed in WO 2009/095479 A2. Accordingly, in certain embodiments the moiety -L¹- is of formula (II):

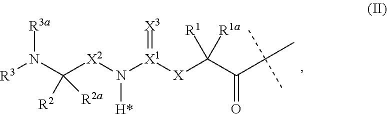

wherein the dashed line indicates attachment to a nitrogen, hydroxyl or thiol of -D; —X— is selected from the group consisting of —C(R⁴R^{4a})—; —N(R⁴)—; —O—; —C(R⁴R^{4a})—C(R⁵R^{5a})—; —C(R⁵R^{5a})—C(R⁴R^{4a})—; —C(R⁴R^{4a})—N(R⁶)—; —N(R⁶)—C(R⁴R^{4a})—.
C(R⁴R^{4a})—O—; —O—C(R⁴R^{4a})—; and —C(R⁷R^{7a})—;
X¹ is selected from the group consisting of C; and S(O);
—X²— is selected from the group consisting of —C(R⁸R^{8a})—; and —C(R⁸R^{8a})—C(R⁹R^{9a})—;
═X³ is selected from the group consisting of ═O; ═S; and ═N—CN;
—R¹, —R^{1a}, —R², —R^{2a}, —R⁴, —R⁵, —R^{5a}, —R⁶, —R⁸, —R^{8a}, —R⁹, and —R^{9a} are independently selected from the group consisting of —H; and C₁₋₆ alkyl;
—R³, and —R^{3a} are independently selected from the group consisting of —H; and C₁₋₆ alkyl, provided that in case one of —R³, —R^{3a} or both are other than —H they are connected to N to which they are attached through an SP³-hybridized carbon atom;
—R⁷ is selected from the group consisting of —N(R¹⁰R^{10a}); and —NR¹⁰—(C═O)—R¹¹;
—R^{7a}, —R¹⁰, —R^{10a}, and —R¹¹ are independently of each other selected from the group consisting of —H; and C₁₋₆ alkyl;
optionally, one or more of the pairs —R^{1a}/—R^{4a}, —R^{1a}/—R^{5a}, —R^{1a}/—R^{7a}, —R^{4a}/—R^{5a}, and —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R⁵/—R^{5a}, and —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R¹/—R⁹, and —R²/—R³ are joined together with the atoms to which they are attached to form a ring A;
optionally, R³/R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with at least one -L²-Z or -L²-Z′ and wherein -L¹- is optionally further substituted, provided that the hydrogen marked with the asterisk in formula (II) is not replaced by -L²-Z or -L²-Z′ or a substituent.

In certain embodiments -L¹- of formula (II) is substituted with one moiety -L²-Z or -L²-Z′.

In certain embodiments -L¹- of formula (II) is not further substituted.

It is understood that if —R³/—R^{3a} of formula (II) are joined together with the nitrogen atom to which they are attached to form a 3- to 10-membered heterocycle, only such 3- to 10-membered heterocycles may be formed in which the atoms directly attached to the nitrogen are SP³-hybridized carbon atoms. In other words, such 3- to 10-membered heterocycle formed by —R³/—R^{3a} together with the nitrogen atom to which they are attached has the following structure:

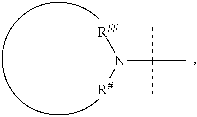

wherein
the dashed line indicates attachment to the rest of -L¹-;
the ring comprises 3 to 10 atoms comprising at least one nitrogen; and
R^{#} and R^{##} represent an sp³-hydridized carbon atom.

It is also understood that the 3- to 10-membered heterocycle may be further substituted.

Exemplary embodiments of suitable 3- to 10-membered heterocycles formed by —R³/—R^{3a} of formula (II) together with the nitrogen atom to which they are attached are the following:

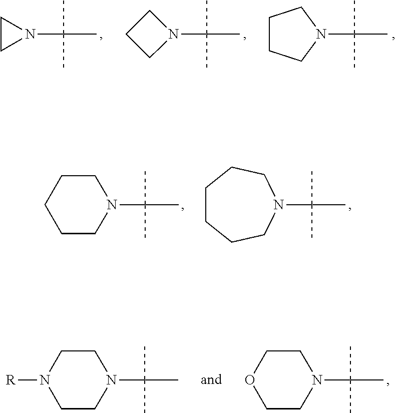

wherein
dashed lines indicate attachment to the rest of the molecule; and
—R is selected from the group consisting of —H and C₁₋₆ alkyl.

-L¹- of formula (II) may optionally be further substituted. In general, any substituent may be used as far as the cleavage principle is not affected, i.e., the hydrogen marked with the asterisk in formula (II) is not replaced and the nitrogen of the moiety

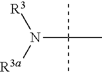

of formula (II) remains part of a primary, secondary or tertiary amine, i.e., —R³ and —R^{3a} are independently of each other —H or are connected to —N<through an sp³-hybridized carbon atom.

In one embodiment —R¹ or —R^{1a} of formula (II) is substituted with -L²-Z or -L²-Z′. In another embodiment —R² or —R^{2a} of formula (II) is substituted with -L²-Z or -L²-Z′. In another embodiment —R³ or —R^{3a} of formula (II) is substituted with -L²-Z or -L²-Z′. In another embodiment —R⁴ of formula (II) is substituted with -L²-Z or -L²-Z′. In another embodiment —R⁵ or —R^{a} of formula (II) is substituted with -L²-Z or -L²-Z′. In another embodiment —R⁶ of formula (II) is substituted with -L²-Z or -L²-Z′. In another embodiment —R⁷ or —R^{7a} of formula (II) is substituted with -L²-Z or -L²-Z′. In another embodiment —R⁸ or —R^{8a} of formula (II) is substituted with -L²-Z or -L²-Z′. In another embodiment —R⁹ or —R^{9a} of formula (II) is substituted with -L²-Z or -L²-Z′. In another embodiment —R¹⁰ is substituted with -L²-Z or -L²-Z′. In another embodiment —R¹¹ is substituted with -L²-Z or -L²-Z′. In certain embodiments —R³ of formula (II) is substituted with -L²-Z or -L²-Z′.

In certain embodiments —X— of formula (II) is selected from the group consisting of —C(R⁴R^{4a})—, —N(R⁴)— and —C(R⁷R^{7a})—. In certain embodiments —X— of formula (II) is —C(R⁴R^{4a})—. In certain embodiments —X— of formula (II) is —C(R⁷R^{7a})—.

In certain embodiments —R⁷ of formula (II) is —NR¹⁰—(C═O)—R¹¹.

In certain embodiments —R^{7a} of formula (II) is selected from —H, methyl and ethyl. In certain embodiments —R^{7a} of formula (II) is —H.

In certain embodiments —R¹⁰ is selected from —H, methyl and ethyl. In certain embodiments —R¹⁰ is methyl.

In certain embodiments —R¹¹ is selected from —H, methyl and ethyl. In certain embodiments —R¹¹ is —H. In certain embodiments —R¹¹ is substituted with -L²-Z or -L²-Z′.

In certain embodiments —X— of formula (II) is —N(R⁴)—.

In certain embodiments —R⁴ is selected from the group consisting of —H, methyl and ethyl. In certain embodiments —R⁴ is —H.

In certain embodiments X¹ of formula (II) is C.

In certain embodiments ═X³ of formula (II) is ═O.

In certain embodiments —X²— of formula (II) is —C(R⁸R^{8a})—.

In certain embodiments —R⁸ and —R^{8a} of formula (II) are independently selected from the group consisting of —H, methyl and ethyl. In certain embodiments at least one of —R⁸ and —R^{8a} of formula (II) is —H. In certain embodiments both —R⁸ and —R^{8a} of formula (II) are —H.

In certain embodiments —R¹ and —R^{1a} of formula (II) are independently selected from the group consisting of —H, methyl and ethyl.

In certain embodiments at least one of —R¹ and —R^{1a} of formula (II) is —H. In certain embodiments —R¹ and —R^{1a} of formula (II) are —H.

In certain embodiments at least one of —R¹ and —R^{1a} of formula (II) is methyl. In certain embodiments both —R¹ and —R^{1a} of formula (II) are methyl.

In certain embodiments —R² and —R^{2a} of formula (II) are independently selected from the group consisting of —H, methyl and ethyl. In certain embodiments at least one of —R² and —R^{2a} of formula (II) is —H. In certain embodiments both —R² and —R^{2a} of formula (II) are H.

In certain embodiments —R³ and —R^{3a} of formula (II) are independently selected from the group consisting of —H, methyl, ethyl, propyl and butyl.

In certain embodiments at least one of —R³ and —R^{3a} of formula (II) is methyl. In certain embodiments —R³ of formula (II) is methyl and —R^{3a} of formula (II) is —H.

In certain embodiments —R³ and —R^{3a} of formula (II) are both —H.

In certain embodiments -D is connected to -L¹- through a nitrogen by forming an amide bond.

In certain embodiments the moiety -L¹- is of formula (IIa-i):

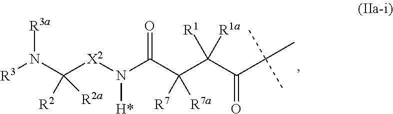

wherein
the dashed line indicates attachment to a nitrogen of -D through an amide bond;
—R¹, —Ria, —R², —R^{2a}, —R³, —R^{3a}, —R⁷, —R^{7a} and —X²— are used as defined in formula (II); and wherein -L¹- is substituted with at least one -L²-Z or -L²-Z′ and wherein -L¹- is optionally further substituted, provided that the hydrogen marked with the asterisk in formula (IIa-i) is not replaced by -L²-Z or -L²-Z′ or a substituent.

It is understood that in case one of —R³, —R^{3a} of formula (IIa-i) or both are other than —H they are connected to N to which they are attached through an sp³-hybridized carbon atom.

In certain embodiments -L¹- of formula (IIa-i) is substituted with one moiety -L²-Z or -L²-Z′.

In certain embodiments the moiety -L¹- of formula (IIa-i) is not further substituted.

In certain embodiments —R¹ and —R^{1a} of formula (IIa-i) are independently selected from the group consisting of —H, methyl and ethyl. In certain embodiments at least one of —R¹ and —R^{1a} of formula (IIa-i) is —H. In certain embodiments both —R¹ and —R^{1a} of formula (IIa-i) are —H.

In certain embodiments —R⁷ of formula (IIa-i) is —NR¹⁰—(C═O)—R¹¹.

In certain embodiments —R^{7a} of formula (II-i) is selected from —H, methyl and ethyl. In certain embodiments —R^{7a} of formula (II-i) is —H.

In certain embodiments —R¹⁰ of formula (IIa-i) is selected from —H, methyl and ethyl. In certain embodiments —R¹⁰ of formula (IIa-i) is methyl.

In certain embodiments —R¹¹ of formula (IIa-i) is selected from —H, methyl and ethyl. In certain embodiments —R¹¹ of formula (IIa-i) is —H.

In certain embodiments —R¹¹ of formula (IIa-i) is substituted with -L²-Z or -L²-Z′.

In certain embodiments —X²— of formula (IIa-i) is —C(R⁸R^{8a})—.

In certain embodiments —R⁸ and —R^{8a} of formula (IIa-i) are independently selected from the group consisting of —H, methyl and ethyl. In certain embodiments at least one of —R⁸ and —R^{8a} of formula (IIa-i) is —H. In certain embodiments both —R⁸ and —R^{8a} of formula (IIa-i) are —H.

In certain embodiments R² and —R^{2a} of formula (IIa-i) are independently selected from the group consisting of —H, methyl and ethyl. In certain embodiments at least one of —R² and —R^{2a} of formula (IIa-i) is —H. In certain embodiments both —R² and —R^{2a} of formula (IIa-i) are H.

In certain embodiments —R³ and —R^{3a} of formula (IIa-i) are independently selected from the group consisting of —H, methyl, ethyl, propyl and butyl. In certain embodiments at least one of —R³ and —R^{3a} of formula (IIa-i) is methyl.

In certain embodiments —R³ of formula (IIa-i) is —H and —R^{3a} of formula (IIa-i) is methyl.

In certain embodiments the moiety -L¹- is of formula (IIa-ii):

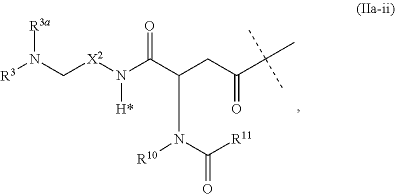

wherein the dashed line indicates attachment to a nitrogen of -D through an amide bond;
—R², —R^{2a}, —R¹⁰, —R¹¹ and —X²— are used as defined in formula (II); and
wherein -L¹- is substituted with at least one -L²-Z or -L²-Z′ and wherein -L¹- is optionally further substituted, provided that the hydrogen marked with the asterisk in formula (IIa-ii) is not replaced by -L²-Z or -L²-Z′ or a substituent.

It is understood that in case one of —R³, —R^{3a} of formula (IIa-ii) or both are other than —H they are connected to N to which they are attached through an sp³-hybridized carbon atom.

In certain embodiments -L¹- of formula (IIa-ii) is substituted with one moiety -L²-Z or -L²-Z′.

In certain embodiments the moiety -L¹- of formula (IIa-ii) is not further substituted.

In certain embodiments —X²— of formula (IIa-ii) is —C(R⁸R^{8a})—.

In certain embodiments —R⁸ and —R^{8a} of formula (IIa-ii) are independently selected from the group consisting of —H, methyl and ethyl. In certain embodiments at least one of —R⁸ and —R^{8a} of formula (IIa-ii) is —H. In certain embodiments both —R⁸ and —R^{8a} of formula (IIa-ii) are —H.

In certain embodiments —R³ and —R^{3a} of formula (IIa-ii) are independently selected from the group consisting of —H, methyl, ethyl, propyl and butyl. In certain embodiments at least one of —R³ and —R^{3a} of formula (IIa-ii) is methyl.

In certain embodiments —R³ of formula (IIa-ii) is —H and —R^{3a} of formula (IIa-ii) is methyl.

In certain embodiments —R¹⁰ of formula (IIa-ii) is selected from —H, methyl and ethyl. In certain embodiments —R¹⁰ of formula (IIa-ii) is methyl.

In certain embodiments —R¹¹ of formula (IIa-ii) is selected from —H, methyl and ethyl. In certain embodiments —R¹¹ of formula (IIa-ii) is —H.

In certain embodiments —R¹¹ of formula (IIa-ii) is substituted with -L²-Z or -L²-Z′.

In certain embodiments the moiety -L¹- is of formula (IIa-ii′):

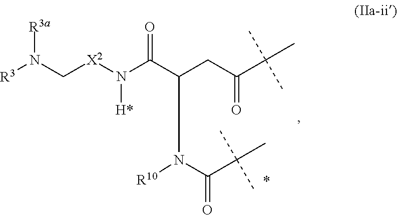

wherein
wherein the dashed line indicates attachment to a nitrogen of -D through an amide bond;
the dashed line marked with the asterisk indicates attachment to -L²-;
—R³, —R^{3a}, —R¹⁰ and —X²— are used as defined in formula (II); and
wherein -L¹- is optionally further substituted, provided that the hydrogen marked with the asterisk in formula (IIa-ii′) is not replaced by a substituent.

It is understood that in case one of —R³, —R^{3a} of formula (IIa-ii′) or both are other than —H they are connected to N to which they are attached through a sp³-hybridized carbon atom.

In certain embodiments —X²— of formula (IIa-ii′) is —C(R⁸R^{8a})—.

In certain embodiments —R⁸ and —R^{8a} of formula (IIa-ii′) are independently selected from the group consisting of —H, methyl and ethyl. In certain embodiments at least one of —R⁸ and —R^{8a} of formula (IIa-ii′) is —H. In certain embodiments both —R⁸ and —R^{8a} of formula (IIa-ii′) are —H.

In certain embodiments —R³ and —R^{3a} of formula (IIa-ii′) are independently selected from the group consisting of —H, methyl, ethyl, propyl and butyl. In certain embodiments at least one of —R³ and —R^{3a} of formula (IIa-ii′) is methyl.

In certain embodiments —R³ of formula (IIa-ii′) is —H and —R^{3a} of formula (IIa-ii′) is methyl.

In certain embodiments —R¹⁰ of formula (IIa-ii′) is selected from —H, methyl and ethyl. In certain embodiments —R¹⁰ of formula (IIa-ii′) is methyl.

In certain embodiments the moiety -L¹- is of formula (IIa-iii):

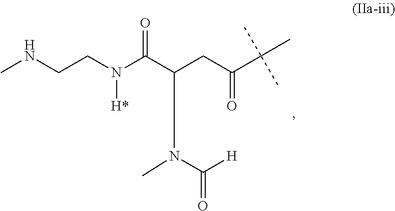

wherein the dashed line indicates attachment to a nitrogen of through an amide bond; and wherein -L¹- is substituted with -L²-Z or -L²-Z′ and wherein -L¹- is optionally further substituted, provided that the hydrogen marked with the asterisk in formula (IIa-iii) is not replaced by -L²-Z or -L²-Z′ or a substituent.

It is understood that in case one of —R³, —R^{3a} of formula (IIa-iii) or both are other than —H they are connected to N to which they are attached through an sp³-hybridized carbon atom.

In certain embodiments the moiety -L¹- of formula (IIa-iii) is not further substituted.

In certain embodiments the moiety -L¹- is of formula (IIa-iii′):

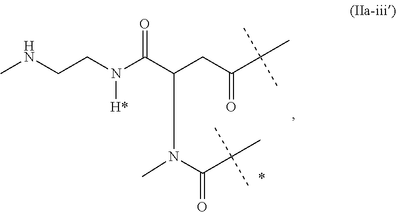

wherein
wherein the dashed line indicates attachment to a nitrogen of -D through an amide bond; the dashed line marked with the asterisk indicates attachment to -L²-;
—R², —R^{2a}, —R³, —R^{3a} and —X²— are used as defined in formula (II); and
wherein -L¹- is optionally further substituted, provided that the hydrogen marked with the asterisk in formula (IIa-iii′) is not replaced by a substituent.

It is understood that in case one of —R³, —R^{3a} of formula (IIa-iii′) or both are other than —H they are connected to N to which they are attached through an sp³-hybridized carbon atom.

In certain embodiments the moiety -L¹- of formula (IIa-iii′) is not further substituted.

In certain embodiments the moiety -L¹- is of formula (IIb-i)

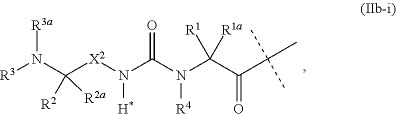

wherein
the dashed line indicates attachment to a nitrogen of -D through an amide bond; —R¹, —Ria, —R², —R^{2a}, —R³, —R^{3a}, —R⁴ and —X²— are used as defined in formula (II); and wherein -L¹- is substituted with at least one -L²-Z or -L²-Z′ and wherein -L¹- is optionally further substituted, provided that the hydrogen marked with the asterisk in formula (IIb-i) is not replaced by -L²-Z or -L²-Z′ or a substituent.

It is understood that in case one of —R³, —R^{3a} of formula (IIb-i) or both are other than —H they are connected to N to which they are attached through an sp³-hybridized carbon atom.

In certain embodiments -L¹- of formula (IIb-i) is substituted with one moiety -L²-Z or -L²-Z′.

In certain embodiments the moiety -L¹- of formula (IIb-i) is not further substituted.

In certain embodiments —R¹ and —R^{1a} of formula (IIb-i) are independently selected from the group consisting of —H, methyl and ethyl. In certain embodiments at least one of —R¹ and —R^{1a} of formula (IIb-i) is methyl. In certain embodiments both —R¹ and —R^{1a} of formula (IIb-i) are methyl.

In certain embodiments —R⁴ of formula (IIb-i) is selected from the group consisting of —H, methyl and ethyl. In certain embodiments —R⁴ of formula (IIb-i) is —H.

In certain embodiments —X²— of formula (IIb-i) is —C(R⁸R^{8a})—.

In certain embodiments —R⁸ and —R^{8a} of formula (IIb-i) are independently selected from the group consisting of —H, methyl and ethyl. In certain embodiments at least one of —R⁸ and —R^{8a} of formula (IIb-i) is —H. In certain embodiments both —R⁸ and —R^{8a} of formula (IIb-i) are —H.

In certain embodiments —R² and —R^{2a} of formula (IIb-i) are independently selected from the group consisting of —H, methyl and ethyl. In certain embodiments at least one of —R² and —R^{2a} of formula (IIb-i) is —H. In certain embodiments both —R² and —R^{2a} of formula (IIb-i) are H.

In certain embodiments —R³ and —R^{3a} of formula (IIb-i) are independently selected from the group consisting of —H, methyl, ethyl, propyl and butyl. In certain embodiments at least one of —R³ and —R^{3a} of formula (IIb-i) is —H. In certain embodiments both —R³ and —R^{3a} of formula (IIb-i) are —H.

In certain embodiments the moiety -L¹- is of formula (IIb-ii):

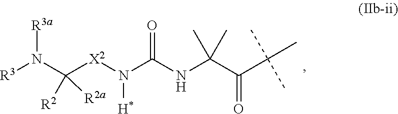

wherein the dashed line indicates attachment to a nitrogen of -D through an amide bond; —R², —R^{2a}, —R³, —R^{3a} and —X²— are used as defined in formula (II); and
wherein -L¹- is substituted with at least one -L²-Z or -L²-Z′ and wherein -L¹- is optionally further substituted, provided that the hydrogen marked with the asterisk in formula (IIb-ii) is not replaced by -L²-Z or -L²-Z′ or a substituent.

It is understood that in case one of —R³, —R^{3a} of formula (IIb-ii) or both are other than —H they are connected to N to which they are attached through an sp³-hybridized carbon atom.

In certain embodiments -L¹- of formula (IIb-ii) is substituted with one moiety -L²-Z or -L²-Z′.

In certain embodiments the moiety -L¹- of formula (IIb-ii) is not further substituted.

In certain embodiments —X²— of formula (IIb-ii) is —C(R⁸R^{8a})—.

In certain embodiments —R⁸ and —R^{8a} of formula (IIb-ii) are independently selected from the group consisting of —H, methyl and ethyl. In certain embodiments at least one of —R⁸ and —R^{8a} of formula (IIb-ii) is —H. In certain embodiments both —R⁸ and —R^{8a} of formula (IIb-ii) are —H.

In certain embodiments —R² and —R^{2a} of formula (IIb-ii) are independently selected from the group consisting of —H, methyl and ethyl. In certain embodiments at least one of —R² and —R^{2a} of formula (IIb-ii) is —H. In certain embodiments both —R² and —R^{2a} of formula (IIb-ii) are H.

In certain embodiments —R³ and —R^{3a} of formula (IIb-ii) are independently selected from the group consisting of —H, methyl, ethyl, propyl and butyl. In certain embodiments at least one of —R³ and —R^{3a} of formula (IIb-ii) is —H. In certain embodiments both —R³ and —R^{3a} of formula (IIb-ii) are —H.

In certain embodiments the moiety -L¹- is of formula (IIb-ii′):

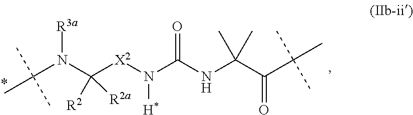

wherein
the dashed line indicates attachment to a nitrogen of -D through an amide bond;
the dashed line marked with the asterisk indicates attachment to -L²-;
—R², —R^{a}, —R^{3a} and —X²— are used as defined in formula (II); and
wherein -L¹- is optionally further substituted, provided that the hydrogen marked with the asterisk in formula (IIb-ii′) is not replaced by a substituent.

It is understood that in case —R^{3a} of formula (IIb-ii′) is other than —H it is connected to N to which it is attached through an sp³-hybridized carbon atom.

In certain embodiments the moiety -L¹- of formula (IIb-ii′) is not further substituted.

In certain embodiments —X²— of formula (IIb-ii′) is —C(R⁸R^{8a})—.

In certain embodiments —R⁸ and —R^{8a} of formula (IIb-ii′) are independently selected from the group consisting of —H, methyl and ethyl. In certain embodiments at least one of —R⁸ and —R^{8a} of formula (IIb-ii′) is —H. In certain embodiments both —R⁸ and —R^{8a} of formula (IIb-ii′) are —H.

In certain embodiments —R² and —R^{2a} of formula (IIb-ii′) are independently selected from the group consisting of —H, methyl and ethyl. In certain embodiments at least one of —R² and —R^{2a} of formula (IIb-ii′) is —H. In certain embodiments both —R² and —R^{2a} of formula (IIb-ii′) are H.

In certain embodiments —R^{3a} of formula (IIb-ii′) is selected from the group consisting of —H, methyl, ethyl, propyl and butyl. In one embodiment —R^{3a} of formula (IIb-ii′) is —H.

In certain embodiments the moiety -L¹- is of formula (IIb-iii):

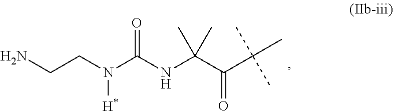

wherein
the dashed line indicates attachment to a nitrogen of -D through an amide bond; and wherein -L¹- is substituted with at least one -L²-Z or -L²-Z′ and wherein -L¹- is optionally further substituted, provided that the hydrogen marked with the asterisk in formula (IIb-iii) is not replaced by -L²-Z or -L²-Z′ or a substituent.

It is understood that in case one of —R³, —R^{3a} of formula (IIb-iii) or both are other than —H they are connected to N to which they are attached through an sp³-hybridized carbon atom.

In certain embodiments -L¹- of formula (IIb-iii) is substituted with one moiety -L²-Z or -L²-Z′.

In certain embodiments the moiety -L¹- of formula (IIb-iii) is not further substituted.

In certain embodiments the moiety -L¹- is of formula (IIb-iii′):

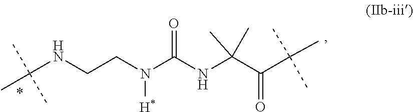

wherein
the dashed line indicates attachment to a nitrogen of -D through an amide bond;
the dashed line marked with the asterisk indicates attachment to -L²-; and
wherein -L¹- is optionally further substituted, provided that the hydrogen marked with the asterisk in formula (IIb-iii′) is not replaced by a substituent.

It is understood that the nitrogen adjacent to the dashed line marked with the asterisk in formula (IIb-iii′) is attached to -L²- through an sp³-hybridized carbon atom.

In certain embodiments the moiety -L¹- of formula (IIb-iii′) is not further substituted.

In certain embodiments -L¹- is disclosed in WO2016/020373A1. Accordingly, in certain embodiments the moiety -L¹- is of formula (III):

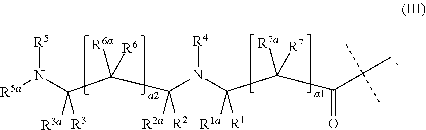

wherein
the dashed line indicates attachment to a primary or secondary amine or hydroxyl of -D through an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R⁸, —C≡N, —C(═NR⁸)R^{8a}, —CR⁸(═CR^{8a}R^{8b}), —C≡CR⁸ and -T;
—R⁴, —R⁵ and —R^{5a} are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R⁸, —R^{8a}, —R^{8b}, —R⁹, —R^{9a}, and —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O) N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}),—OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—,—S—, —N(R¹²)—, —OC(OR¹²)(R^{12a})—, —N(R¹²)C(O)N(R^{12a})—, and —OC(O)N(R¹²)—; each —R¹⁰, —R^{10a}, and —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³, —R^{13a}, and —R^{13b} is independently selected from the group consisting of —H, and C₁₋₆ alkyl; wherein C₁₋₆ alkyl is optionally substituted with one or more halogen, which are the same or different;
optionally, one or more of the pairs —R/—R^{1a}, —R²/—R^{2a}, —R³/—R^{3a}, —R⁶/—R^{6a}, and —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, and —R⁶/—R⁷ are joint together with
the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein -L¹- is substituted with at least one -L²-Z or -L²-Z′ and wherein -L¹- is optionally further substituted.

The optional further substituents of -L¹- of formula (III) are in certain embodiments as described above.

In certain embodiments -L¹- of formula (III) is substituted with one moiety -L²-Z or -L²-Z′.

In certain embodiments -L¹- of formula (III) is not further substituted.

In certain embodiments -L¹- is as disclosed in EP1536334B1, WO2009/009712A1, WO2008/034122A1, WO2009/143412A2, WO2011/082368A2, and U.S. Pat. No. 8,618,124B2, which are herewith incorporated by reference in their entirety.

In certain embodiments -L¹- is as disclosed in U.S. Pat. No. 8,946,405B2 and U.S. Pat. No. 8,754,190B2, which are herewith incorporated by reference in their entirety. Accordingly, in certain embodiments -L¹- is of formula (IV):

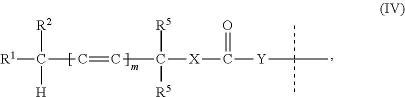

wherein
the dashed line indicates attachment to -D and wherein attachment is through a functional group of -D selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴, one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and
optionally, both —R⁹ together with the nitrogen to which they are attached form a heterocyclic ring;
wherein -L¹- is substituted with at least one -L²-Z or -L²-Z′ and wherein -L¹- is optionally further substituted.

Only in the context of formula (IV) the terms used have the following meaning:

The term “alkyl” as used herein includes linear, branched or cyclic saturated hydrocarbon groups of 1 to 8 carbons, or in certain embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, such as 6 to 10 carbons, including groups such as phenyl, naphthyl, and anthracenyl. The term “heteroaryl” includes aromatic rings comprising 3 to 15 carbons containing at least one N, O or S atom, such as 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted. Optional substituents on any group, including the above, include halo, nitro, cyano, —OR, —SR, —NR₂, —OCOR, —NRCOR, —COOR, —CONR₂, —SOR, —SO₂R, —SONR₂, —SO₂N R₂, wherein each R is independently alkyl, alkenyl, alkynyl, aryl or heteroaryl, or two R groups taken together with the atoms to which they are attached form a ring.

In certain embodiments -L¹- of formula (IV) is substituted with one moiety -L²-Z or -L²-Z′.

In certain embodiments -L¹- of formula (IV) is not further substituted.

In certain embodiments -L¹- is as disclosed in WO2013/036857A1, which is herewith incorporated by reference in its entirety. Accordingly, in certain embodiments -L¹- is of formula (V):

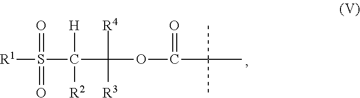

wherein
the dashed line indicates attachment to -D through an amine functional group of -D;
—R¹ is selected from the group consisting of optionally substituted C₁-C₆ linear, branched, or cyclic alkyl; optionally substituted aryl; optionally substituted heteroaryl; alkoxy; and —NR⁵₂;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl;
wherein -L¹- is substituted with at least one -L²-Z or -L²-Z′ and wherein -L¹- is optionally further substituted.

Only in the context of formula (V) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, such as 6-10 carbons, including groups such as phenyl, naphthyl, and anthracene “Heteroaryl” includes aromatic rings comprising 3-15 carbons containing at least one N, O or S atom, such as 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

In certain embodiments -L¹- of formula (V) is substituted with one moiety -L²-Z or -L²-Z′.

In certain embodiments -L¹- of formula (V) is not further substituted.

In certain embodiments -L¹- is as disclosed in U.S. Pat. No. 7,585,837B2, which is herewith incorporated by reference in its entirety. Accordingly, in certain embodiments -L¹- is of formula (VI):

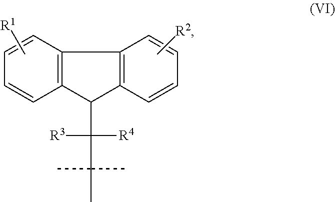

wherein
the dashed line indicates attachment to -D through an amine functional group of -D; R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with at least one -L²-Z or -L²-Z′ and wherein -L¹- is optionally further substituted.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (VI) the terms used have the following meaning: The terms “alkyl”, “alkoxy”, “alkoxyalkyl”, “aryl”, “alkaryl” and “aralkyl” mean alkyl radicals of 1-8, such as 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

In certain embodiments -L¹- of formula (VI) is substituted with one moiety -L²-Z or -L²-Z′.

In certain embodiments -L¹- of formula (VI) is not further substituted.

A further preferred embodiment for -L¹- is disclosed in WO2002/089789A1, which is herewith incorporated by reference in its entirety. Accordingly, a preferred moiety -L¹- is of formula (VII):

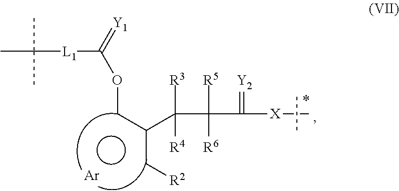

wherein
the dashed line indicates attachment to -D through an amine functional group of -D;
L₁ is a bifunctional linking group,
Y₁ and Y₂ are independently O, S or NR₇;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with at least one -L²-Z or -L²-Z′ and wherein -L¹- is optionally further substituted.

Only in the context of formula (VII) the terms used have the following meaning:

The term “alkyl” shall be understood to include, e.g., straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moeities such as methoxy; and phenoxy includes moieties such as 3-nitrophenoxy. Halo- shall be understood to include fluoro, chloro, iodo and bromo.

In certain embodiments -L¹- of formula (VII) is substituted with one moiety -L²-Z or -L²-Z′.

In certain embodiments -L¹- of formula (VII) is not further substituted.

In certain embodiments -L¹- comprises a substructure of formula (VIII)

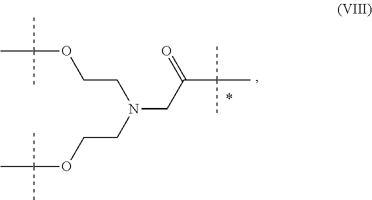

wherein
the dashed line marked with the asterisk indicates attachment to a nitrogen of -D through an amide bond;
the unmarked dashed lines indicate attachment to the remainder of -L¹-; and
wherein -L¹- is substituted with at least one -L²-Z or -L²-Z′ and wherein -L¹- is optionally further substituted.

In certain embodiments -L¹- of formula (VIII) is substituted with one moiety -L²-Z or -L²-Z′.

In certain embodiments -L¹- of formula (VIII) is not further substituted.

In certain embodiments -L¹- comprises a substructure of formula (IX)

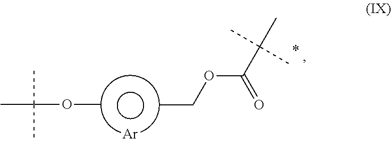

wherein
the dashed line marked with the asterisk indicates attachment to a nitrogen of -D through a carbamate bond;
the unmarked dashed lines indicate attachment to the remainder of -L¹-; and
wherein -L¹- is substituted with at least one -L²-Z or -L²-Z′ and wherein -L¹- is optionally further substituted.

In certain embodiments -L¹- of formula (IX) is substituted with one moiety -L²-Z or -L²-Z′.

In certain embodiments -L¹- of formula (IX) is not further substituted.

In certain embodiments -L¹- has a structure as disclosed in WO2020/206358 A1. Accordingly, in certain embodiments the moiety -L¹- is of formula (X):

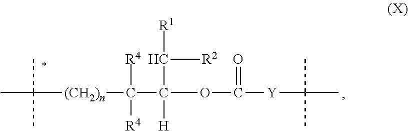

wherein
the unmarked dashed line indicates attachment to -D;
the dashed line marked with the asterisk indicates attachment to -L²-Z or -L²-Z′;
n is an integer selected from the group consisting of 0, 1, 2, 3, 4, 5 and 6;
—R¹ and —R² are independently an electron-withdrawing group, alkyl, or —H, and wherein at least one of —R¹ or —R² is an electron-withdrawing group;
each —R⁴ is independently C₁-C₃ alkyl or the two —R⁴ are taken together with the carbon atom to which they are attached to form a 3- to 6-membered ring; and
—Y— is absent when -D is a drug moiety connected through an amine, or —Y— is —N(R⁶)CH₂— when -D is a drug moiety connected through a phenol, alcohol, thiol, thiophenol, imidazole, or non-basic amine; wherein —R⁶ is optionally substituted C₁-C₆ alkyl, optionally substituted aryl, or optionally substituted heteroaryl.

In certain embodiments n of formula (X) is an integer selected from 1, 2, 3, 4, 5 and 6. In certain embodiments n of formula (X) is an integer selected from 1, 2 and 3. In certain embodiments n of formula (X) is an integer from 0, 1, 2 and 3. In certain embodiments n of formula (X) is 1. In certain embodiments n of formula (X) is 2. In certain embodiments n of formula (X) is 3.

In certain embodiments the electron-withdrawing group of —R¹ and —R² of formula (X) is selected from the group consisting of —CN; —NO₂; optionally substituted aryl; optionally substituted heteroaryl; optionally substituted alkenyl; optionally substituted alkynyl; —COR³, —SOR³, or —SO₂R³, wherein —R³ is —H, optionally substituted alkyl, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl, or both —R⁸ groups are taken together with the nitrogen to which they are attached to form a heterocyclic ring; or —SR⁹, wherein —R⁹ is optionally substituted alkyl, optionally substituted aryl, optionally substituted arylalkyl, optionally substituted heteroaryl, or optionally substituted heteroarylalkyl.

In certain embodiments the electron-withdrawing group of —R¹ and —R² of formula (X) is —CN. In certain embodiments the electron-withdrawing group of —R¹ and —R² of formula (X) is —NO₂. In certain embodiments the electron-withdrawing group of —R¹ and —R² of formula (X) is optionally substituted aryl comprising 6 to 10 carbons. In certain embodiments the electron-withdrawing group of —R¹ and —R² of formula (X) is optionally substituted phenyl, naphthyl, or anthracenyl. In certain embodiments the electron-withdrawing group of —R¹ and —R² of formula (X) is optionally substituted heteroaryl comprising 3 to 7 carbons and comprising at least one N, O, or S atom. In certain embodiments the electron-withdrawing group of —R¹ and —R² of formula (X) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments the electron-withdrawing group of —R¹ and —R² of formula (X) is optionally substituted alkenyl containing 2 to 20 carbon atoms. In certain embodiments the electron-withdrawing group of —R¹ and —R² of formula (X) is optionally substituted alkynyl comprising 2 to 20 carbon atoms. In certain embodiments the electron-withdrawing group of —R¹ and —R² of formula (X) is —COR³, —SOR³, or —SO₂R³, wherein —R³ is —H, optionally substituted alkyl comprising 1 to 20 carbon atoms, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl comprising 1 to 20 carbon atoms, or both —R⁸ groups are taken together with the nitrogen to which they are attached to form a heterocyclic ring. In certain embodiments the electron-withdrawing group of —R¹ and —R² of formula (X) is —SR⁹, wherein —R⁹ is optionally substituted alkyl comprising 1 to 20 carbon atoms, optionally substituted aryl, optionally substituted arylalkyl, optionally substituted heteroaryl, or optionally substituted heteroarylalkyl.

In certain embodiments at least one of —R¹ or —R² of formula (X) is —CN, —SOR³ or —SO₂R³. In certain embodiments at least one of —R¹ and —R² of formula (X) is —CN or —SO₂R³. In certain embodiments at least one of —R¹ and —R² of formula (X) is —CN or —SO₂R³, wherein —R³ is optionally substituted alkyl, optionally substituted aryl, or —NR⁸₂. In certain embodiments at least one of —R¹ and —R² of formula (X) is —CN, —SO₂N(CH₃)₂, —SO₂CH₃, phenyl substituted with —SO₂, phenyl substituted with —SO₂ and —Cl, —SO₂N(CH₂CH₂)₂O, —SO₂CH(CH₃)₂, —SO₂N(CH₃)(CH₂CH₃), or —SO₂N(CH₂CH₂OCH₃)₂.

In certain embodiments each —R⁴ of formula (X) is independently C₁-C₃ alkyl. In certain embodiments both —R⁴ are methyl.

In certain embodiments —Y— of formula (X) is absent. In certain embodiments —Y— of formula (X) is —N(R⁶)CH₂—.

In certain embodiments -L¹- is of formula (X), wherein n is 1, —R¹ is —CN, —R² is —H, and —R⁴ is —CH₃. In certain embodiments -L¹- is of formula (X), wherein n is 1, —R¹ is —SO₂N(CH₃)₂, —R² is —H, and —R⁴ is —CH₃. In certain embodiments -L¹- is of formula (X), wherein n is 1, —R¹ is SO₂CH₃, —R² is —H, and —R⁴ is —CH₃. In certain embodiments -L¹- is of formula (X), wherein n is 1, —R¹ is —SO₂N(CH₂CH₂)₂CHCH₃, —R² is —H, and —R⁴ is —CH₃. In certain embodiments -L¹- is of formula (X), wherein n is 1, —R¹ is phenyl substituted with —SO₂, —R² is —H, and —R⁴ is —CH₃.

In certain embodiments -L¹- is of formula (X), wherein n is 1, —R¹ is phenyl substituted with —SO₂ and —Cl, —R² is —H, and —R⁴ is —CH₃. In certain embodiments -L¹- is of formula (X), wherein n is 1, —R¹ is —SO₂N(CH₂CH₂)₂O, —R² is —H, and —R⁴ is —CH₃. In certain embodiments -L¹- is of formula (X), wherein n is 1, —R¹ is —SO₂CH(CH₃)₂, —R² is —H, and —R⁴ is —CH₃. In certain embodiments -L¹- is of formula (X), wherein n is 1, —R¹ is —SO₂N(CH₃)(CH₂CH₃), —R² is —H, and —R⁴ is —CH₃. In certain embodiments -L¹- is of formula (X), wherein n is 1, —R¹ is —SO₂N(CH₂CH₂OCH₃)₂, —R² is —H, and —R⁴ is —CH₃. In certain embodiments -L¹- is of formula (X), wherein n is 1, —R¹ is phenyl substituted with —SO₂ and —CH₃, —R² is —H, and —R⁴ is —CH₃.

In certain embodiments -L¹- is of formula (X), wherein n is 2, —R¹ is —CN, —R² is —H, and —R⁴ is —CH₃. In certain embodiments -L¹- is of formula (X), wherein n is 2, —R¹ is —SO₂N(CH₃)₂, —R² is —H, and —R⁴ is —CH₃. In certain embodiments -L¹- is of formula (X), wherein n is 2, —R¹ is SO₂CH₃, —R² is —H, and —R⁴ is —CH₃. In certain embodiments -L¹- is of formula (X), wherein n is 2, —R¹ is —SO₂N(CH₂CH₂)₂CHCH₃, —R² is —H, and —R⁴ is —CH₃. In certain embodiments -L¹- is of formula (X), wherein n is 2, —R¹ is phenyl substituted with —SO₂, —R² is —H, and —R⁴ is —CH₃.

In certain embodiments -L¹- is of formula (X), wherein n is 2, —R¹ is phenyl substituted with —SO₂ and —Cl, —R² is —H, and —R⁴ is —CH₃. In certain embodiments -L¹- is of formula (X), wherein n is 2, —R¹ is —SO₂N(CH₂CH₂)₂O, —R² is —H, and —R⁴ is —CH₃. In certain embodiments -L¹- is of formula (X), wherein n is 2, —R¹ is —SO₂CH(CH₃)₂, —R² is —H, and —R⁴ is —CH₃. In certain embodiments -L¹- is of formula (X), wherein n is 2, —R¹ is —SO₂N(CH₃)(CH₂CH₃), —R² is —H, and —R⁴ is —CH₃. In certain embodiments -L¹- is of formula (X), wherein n is 2, —R¹ is —SO₂N(CH₂CH₂OCH₃)₂, —R² is —H, and —R⁴ is —CH₃. In certain embodiments -L¹- is of formula (X), wherein n is 2, —R¹ is phenyl substituted with —SO₂ and —CH₃, —R² is —H, and —R⁴ is —CH₃.

In certain embodiments -L¹- is of formula (X), wherein n is 3, —R¹ is —CN, —R² is —H, and —R⁴ is —CH₃. In certain embodiments -L¹- is of formula (X), wherein n is 3, —R¹ is —SO₂N(CH₃)₂, —R² is —H, and —R⁴ is —CH₃. In certain embodiments -L¹- is of formula (X), wherein n is 3, —R¹ is SO₂CH₃, —R² is —H, and —R⁴ is —CH₃. In certain embodiments -L¹- is of formula (X), wherein n is 3, —R¹ is —SO₂N(CH₂CH₂)₂CHCH₃, —R² is —H, and —R⁴ is —CH₃. In certain embodiments -L¹- is of formula (X), wherein n is 3, —R¹ is phenyl substituted with —SO₂, —R² is —H, and —R⁴ is —CH₃.

In certain embodiments -L¹- is of formula (X), wherein n is 3, —R¹ is phenyl substituted with —SO₂ and —Cl, —R² is —H, and —R⁴ is —CH₃. In certain embodiments -L¹- is of formula (X), wherein n is 3, —R¹ is —SO₂N(CH₂CH₂)₂O, —R² is —H, and —R⁴ is —CH₃. In certain embodiments -L¹- is of formula (X), wherein n is 3, —R¹ is —SO₂CH(CH₃)₂, —R² is —H, and —R⁴ is —CH₃. In certain embodiments -L¹- is of formula (X), wherein n is 3, —R¹ is —SO₂N(CH₃)(CH₂CH₃), —R² is —H, and —R⁴ is —CH₃. In certain embodiments -L¹- is of formula (X), wherein n is 3, —R¹ is —SO₂N(CH₂CH₂OCH₃)₂, —R² is —H, and —R⁴ is —CH₃. In certain embodiments -L¹- is of formula (X), wherein n is 3, —R¹ is phenyl substituted with —SO₂ and —CH₃, —R² is —H, and —R⁴ is —CH₃.

Only in the context of formula (X) the terms used have the following meaning: The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n- octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments alkenyl, alkynyl, aryl or heteroaryl moieties may be coupled to the 30 remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments a heterocyclic ring or heterocyclyl is non-aromatic. In certain embodiments a heterocyclic ring or heterocyclyl is aromatic.

The term “optionally substituted” refers to a group may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each —R^{cc} is independently alkyl, alkenyl, alkynyl, halogen, heterocyclyl, heteroaryl, aryl, —CN, or —NO₂.

In certain embodiments -L²- is a chemical bond. In certain embodiments -L²- is a spacer moiety, such as a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3}) S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R⁵R^{y5a}), —S(O)₂N(R⁵R^{y5a}), —S(O)N(R⁵R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R⁵R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5}a and —R^{y5b} is independently selected from the group consisting of —H, and C₁₋₆ alkyl, wherein C₁₋₆ alkyl is optionally substituted with one or more halogen, which are the same or different.

In certain embodiments -L²- is selected from -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, oxo (═O), —COOR^{y5}, —OR^{y5}, —C(O)R^{y5}, —C(O)N(R_{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R⁵R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;
each —R^{y2} is independently selected from the group consisting of halogen, and C₁₋₆ alkyl; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6}a are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments -L²- has a molecular weight in the range of from 14 g/mol to 750 g/mol.

In certain embodiments -L²- comprises a moiety selected from

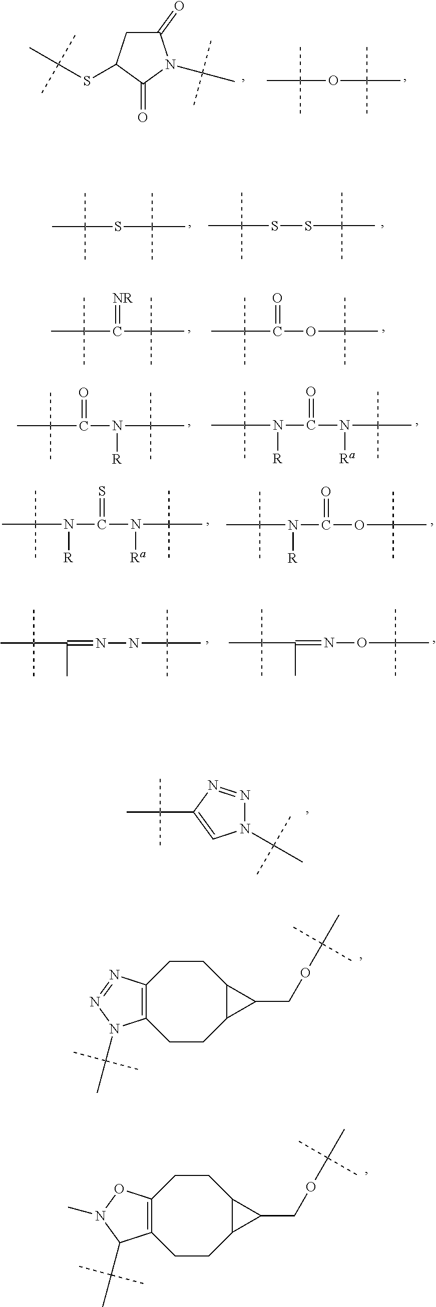

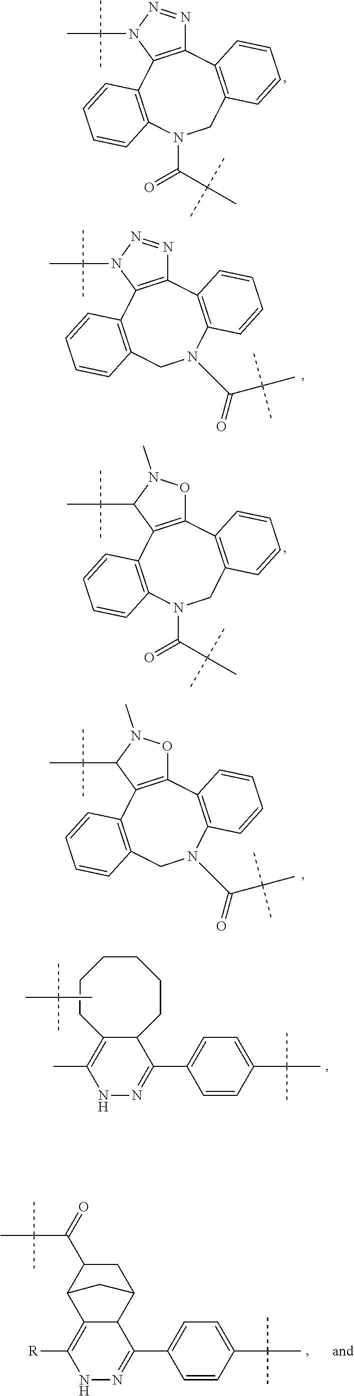

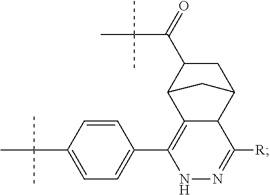

wherein
dashed lines indicate attachment to the remainder of -L²-, -L¹-, —Z and/or Z′, respectively; and —R and —R^{a} are independently of each other selected from the group consisting of —H, methyl, ethyl, propyl, butyl, pentyl and hexyl.

In certain embodiments -L²- has a chain length of 1 to 20 atoms.

As used herein the term “chain length” with regard to the moiety -L²- refers to the number of atoms of -L²- present in the shortest connection between -L¹- and —Z.

In certain embodiments -L²- is of formula (i)

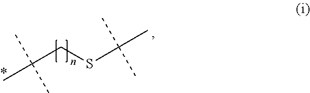

wherein
the dashed line marked with the asterisk indicates attachment to -L¹-;
the unmarked dashed line indicates attachment to —Z or —Z′;
n is selected from the group consisting of 0, 1, 2, 3, 4, 5, 6, 7, 8, 9, 10, 11, 12, 13, 14, 15, 16, 17 and 18; and
wherein the moiety of formula (i) is optionally further substituted.

In certain embodiments n of formula (i) is selected from the group consisting of 3, 4, 5, 6, 7, 8, and 9. In certain embodiments n of formula (i) is 4, 5, 6, or 7. In certain embodiments n of formula (i) is 4. In certain embodiments n of formula (i) is 5. In certain embodiments n of formula (i) is 6.

In certain embodiments the moiety -L¹-L²- is selected from the group consisting of

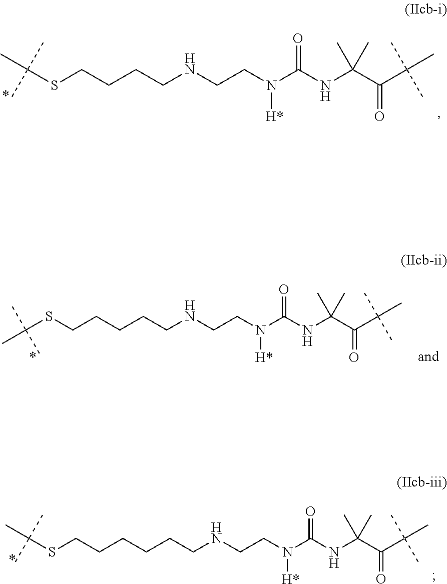

wherein
the unmarked dashed line indicates the attachment to a nitrogen of -D by forming an amide bond; and
the dashed line marked with the asterisk indicates attachment to —Z or Z′.

In certain embodiments the moiety -L¹-L²- is of formula (IIca-ii). In certain embodiments the moiety -L¹-L²- is of formula (IIcb-iii).

In certain embodiments the moiety -L¹-L²- is selected from the group consisting of

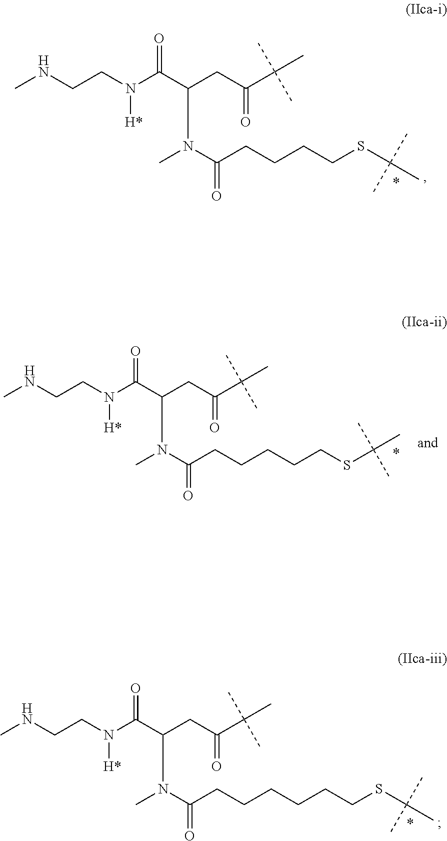

wherein
the unmarked dashed line indicates the attachment to a nitrogen of -D by forming an amide bond; and
the dashed line marked with the asterisk indicates attachment to —Z or Z′.

The carrier —Z comprises a C₈₋₂₄ alkyl or a polymer. In certain embodiments —Z comprises a polymer, such as a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof. In certain embodiments —Z comprises poly(ethylene glycol) (PEG).

In certain embodiments —Z has a molecular weight ranging from 5 to 200 kDa. In certain embodiments —Z has a molecular weight ranging from 8 to 100 kDa, such as ranging from 10 to 80 kDa, from 12 to 60 kDa, or from 15 to 40 kDa. In certain embodiments —Z has a molecular weight of about 20 kDa. In certain embodiments —Z has a molecular weight of about 40 kDa.

In certain embodiments —Z comprises PEG and has a molecular weight ranging from 5 to 200 kDa. In certain embodiments —Z comprises PEG and has a molecular weight ranging from 8 to 100 kDa, such as ranging from 10 to 80 kDa, from 12 to 60 kDa, or from 15 to 40 kDa. In certain embodiments —Z comprises PEG and has a molecular weight of about 20 kDa. In certain embodiments —Z comprises PEG and has a molecular weight of about 40 kDa.

In certain embodiments —Z comprises a protein, such as a protein selected from the group consisting of carboxyl-terminal polypeptide of the chorionic gonadotropin as described in US 2012/0035101 A1 which are herewith incorporated by reference; albumin; XTEN sequences as described in WO 2011123813 A2 which are herewith incorporated by reference; proline/alanine random coil sequences as described in WO 2011/144756 A1 which are herewith incorporated by reference; proline/alanine/serine random coil sequences as described in WO 2008/155134 A1 and WO 2013/024049 A1 which are herewith incorporated by reference; and Fc fusion proteins. In certain embodiments —Z is a polysarcosine. In certain embodiments —Z comprises a poly(N-methylglycine). In certain embodiments —Z comprises a random coil protein moiety.

In certain embodiments —Z comprises a fatty acid derivate, such as a derivative as disclosed in WO 2005/027978 A2 and WO 2014/060512 A1 which are herewith incorporated by reference. In certain embodiments —Z is a hyaluronic acid-based polymer. In certain embodiments —Z is a carrier as disclosed in WO 2012/02047 A1 which is herewith incorporated by reference. In certain embodiments —Z is a carrier as disclosed in WO 2013/024048 A1 which is herewith incorporated by reference. In certain embodiments —Z is a PEG-based polymer, such as a linear, branched or multi-arm PEG-based polymer. In certain embodiments —Z is a linear PEG-based polymer. In certain embodiments —Z is a multi-arm PEG-based polymer. In certain embodiments —Z is a multi-arm PEG-based polymer having at least 4 PEG-based arms.

In certain embodiments such multi-arm PEG-based polymer —Z is connected to a multitude of moieties -L²-L¹-D, wherein each moiety -L²-L¹-D is in certain embodiments connected to the end of an arm. In certain embodiments such multi-arm PEG-based polymer —Z is connected to 2, 3, 4, 5, 6, 7, 8, 9, 10, 11, 12, 13, 14, 15 or 16 moieties -L²-L¹-D. In certain embodiments such multi-arm PEG-based polymer —Z is connected to 2, 3, 4, 6 or 8 moieties -L²-L¹-D. In certain embodiments such multi-arm PEG-based polymer —Z is connected to 2, 4 or 6 moieties -L²-L¹-D. In certain embodiments such multi-arm PEG-based polymer —Z is connected to 4 or 6 moieties -L²-L¹-D. In certain embodiments such multi-arm PEG-based polymer —Z is connected to 4 moieties -L²-L¹-D.

In certain embodiments —Z is a branched PEG-based polymer. In certain embodiments —Z is a branched PEG-based polymer having one, two, three, four, five or six branching points. In certain embodiments —Z is a branched PEG-based polymer having one, two or three branching points. In certain embodiments —Z is a branched PEG-based polymer having one branching point. In certain embodiments —Z is a branched PEG-based polymer having two branching points. In certain embodiments —Z is a branched PEG-based polymer having three branching points. In certain embodiments a branching point may be selected from the group consisting of —N<, —CH< and >C<.

In certain embodiments —Z is a branched PEG-based polymer with one branching point and a molecular weight ranging from 5 to 200 kDa. In certain embodiments —Z is a branched PEG-based polymer with one branching point and a molecular weight ranging from 8 to 100 kDa. In certain embodiments —Z is a branched PEG-based polymer with one branching point and a molecular weight ranging from 10 to 80 kDa. In certain embodiments —Z is a branched PEG-based polymer with one branching point and a molecular weight ranging from 12 to 60 kDa. In certain embodiments —Z is a branched PEG-based polymer with one branching point and a molecular weight ranging from 15 to 40 kDa. In certain embodiments —Z is a branched PEG-based polymer with one branching point and a molecular weight of approx. 20 kDa. In certain embodiments —Z is a branched PEG-based polymer with one branching point and a molecular weight of approx. 40 kDa.

In certain embodiments —Z is a branched PEG-based polymer with one branching point, which branching point is —CH<, and a molecular weight of approx. 40 kDa.

In certain embodiments —Z or Z′ comprises a moiety

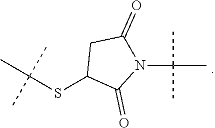

In certain embodiments —Z or Z′ comprises an amide bond.

In certain embodiments-Z comprises a moiety of formula (a)

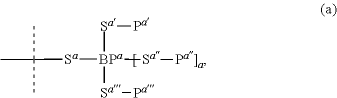

wherein
the dashed line indicates attachment to -L²- or to the remainder of —Z;
BP^{a} is a branching point selected from the group consisting of —N<, —CR< and >C<; —R is selected from the group consisting of —H and C₁₋₆ alkyl;
a is 0 if BP^{a} is —N< or —CR< and n is 1 if BP^{a} is >C<; —S^{a}—, —S^{a′}—, —S^{a″}— and —S^{a′″}— are independently of each other a chemical bond or are selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different;
each —R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR³a, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different; and
—P^{a′}, —P^{a″} and —P^{a′″} are independently a polymeric moiety.

In certain embodiments BP^{a} of formula (a) is —N<. In certain embodiments BP^{a} of formula (a) is >C<. In certain embodiments BP^{a} of formula (a) is —CR<. In certain embodiments —R is —H. Accordingly, a of formula (a) is 0.

In certain embodiments —S^{a}—of formula (a) is a chemical bond. In certain embodiments —S^{a}—of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of -T-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein -T- is a 3- to 10-membered heterocyclyl; and —R⁴ and —R^{4a} are independently selected from the group consisting of —H, methyl, ethyl, propyl and butyl. In certain embodiments —S^{a}—of formula (a) is selected from the group consisting of C₁₋₁₀ alkyl which is interrupted by one or more chemical groups selected from the group consisting of -T-, —C(O)N(R⁴)—and —O—.

In certain embodiments —S^{a′}—of formula (a) is a chemical bond. In certain embodiments —S^{a′}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments —S^{a′}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments —S^{a″}— of formula (a) is a chemical bond. In certain embodiments —S^{a″}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments —S^{a′″}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments —S^{a′″}—of formula (a) is a chemical bond. In certain embodiments —S^{a′″}—of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments —S^{a′″}—of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments —P^{a′}, —P^{a″} and —P^{a′″} of formula (a) independently comprise a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments —P^{a′}, —P^{a″} and —P^{a′″} of formula (a) independently comprise a PEG-based moiety. In certain embodiments —P^{a′}, —P^{a″} and —P^{a′″} of formula (a) independently comprise a PEG-based moiety comprising at least 20% PEG, such as at least 30%, such as at least 40% PEG, such as at least 50% PEG, such as at least 60% PEG, such as at least 70% PEG, such as at least 80% PEG or such as at least 90% PEG.

In certain embodiments —P^{a′}, —P^{a″} and —P^{a′″} of formula (a) independently have a molecular weight ranging from and including 5 kDa to 50 kDa, such as from and including 5 kDa to 40 kDa, such as from and including 7.5 kDa to 35 kDa, such as from and 7.5 to 30 kDa or such as from and including 10 to 30 kDa. In certain embodiments —P^{a′}, —P^{a″} and —P^{a′″} of formula (a) have a molecular weight of about 5 kDa. In certain embodiments —P^{a′}, —P^{a″} and —P^{a′″} of formula (a) have a molecular weight of about 7.5 kDa. In certain embodiments —P^{a′}, —P^{a″} and —P^{a′″} of formula (a) have a molecular weight of about 10 kDa. In certain embodiments —P^{a′}, —P^{a″} and —P^{a′″} of formula (a) have a molecular weight of about 12.5 kDa. In certain embodiments —P^{a′}, —P^{a″} and —P^{a′″} of formula (a) have a molecular weight of about 15 kDa. In certain embodiments —P^{a′}, —P^{a″} and —P^{a′″} of formula (a) have a molecular weight of about 20 kDa.

In certain embodiments —Z comprises one moiety of formula (a). In certain embodiments —Z comprises two moieties of formula (a). In certain embodiments —Z comprises three moieties of formula (a). In certain embodiments —Z is a moiety of formula (a).

In certain embodiments —Z comprises a moiety of formula (b)

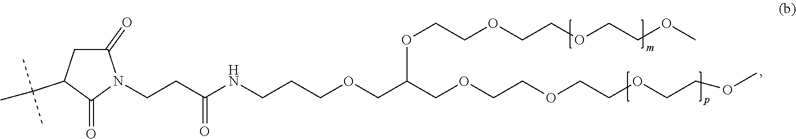

wherein
the dashed line indicates attachment to -L²- or to the remainder of —Z; and m and p are independently of each other an integer ranging from and including 150 to 1000; such as an integer ranging from and including 150 to 500; such as an integer ranging from and including 200 to 500; or such as an integer ranging from and including 400 to 500.

In certain embodiments m and p of formula (b) are the same integer. In certain embodiments m and p of formula (b) are about 450.

In certain embodiments —Z is a moiety of formula (b).

In certain embodiments, Z′ is a hydrogel.

In certain embodiments, Z′ is a PEG-based or hyaluronic acid-based hydrogel. In certain embodiments, Z′ is a PEG-based hydrogel. In certain embodiments, Z′ is a hyaluronic acid-based hydrogel.

In certain embodiments, Z′ is a hydrogel as described in WO 2006/003014 A2, WO 2011/012715 A1, WO 2014/056926 A1, WO2020/064846 or WO2020/064847 which are herewith incorporated by reference in their entirety.

In certain embodiments, Z′ is a hydrogel as disclosed in WO 2013/036847 A1. In particular, in certain embodiments, Z′ is a hydrogel produced by a method comprising the step of reacting at least a first reactive polymer with a cleavable crosslinker compound, wherein said cleavable crosslinker compound comprises a first functional group —Y¹ that reacts with the first reactive polymer and further comprises a moiety that is cleaved by elimination under physiological conditions wherein said moiety comprises a second functional group —Y² that reacts with a second reactive polymer. In certain embodiments, the cleavable crosslinker compound is of formula (PL-1):

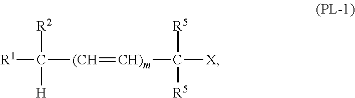

wherein
m is 0 or 1;
—X comprises a functional group capable of connecting to a reactive polymer that is amenable to elimination under physiological conditions and said second functional group —Y²
at least one of —R¹, —R² and —R⁵ comprises said first functional group —Y¹ capable of connecting to a polymer;
one and only one of —R¹ and —R² is selected from the group consisting of —H, alkyl, arylalkyl, and heteroarylalkyl;
optionally, —R¹ and —R² may be joined to form a 3- to 8-membered ring;
at least one or both of —R¹ and —R² is independently selected from the group consisting of —CN, —NO₂, aryl, heteroaryl, alkenyl, alkynyl, —COR³, —SOR³, —SO₂R³ and —SR⁴;
—R³ is selected from the group consisting of —H, alkyl, aryl, arylalkyl, heteroaryl, heteroarylalkyl, —OR⁹ and —NR⁹²;
—R⁴ is selected from the group consisting of alkyl, aryl, arylalkyl, heteroaryl and heteroarylalkyl;
each —R⁵ is independently selected from the group consisting of —H, alkyl, alkenylalkyl, alkynylalkyl, (OCH₂CH₂)ₚO-alkyl with p being an integer ranging from 1 to 1000, aryl, arylalkyl, heteroaryl and heteroarylalkyl;
each —R⁹ is independently selected from the group consisting of —H and alkyl or both —R⁹ together with the nitrogen to which they are attached form a heterocyclic ring; and wherein the moiety of formula (PL-1) is optionally further substituted.

The following paragraphs describe such hydrogel in more detail.

In certain embodiments, —X of formula (PL-1) is selected from the group consisting of succinimidyl carbonate, sulfosuccinimidyl carbonate halides, thioethers, esters, nitrophenyl carbonate, chloroformate, fluoroformate, optionally substituted phenols and formula (PL-2):

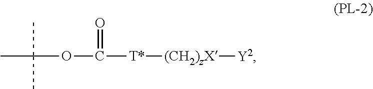

wherein
the dashed line indicates attachment to the remainder of formula (PL-1);
-T*- is selected from the group consisting of —O—, —S— and —NR⁶—;
z is an integer selected from the group consisting of 1, 2, 3, 4, 5 and 6;
—X′— is absent or is selected from the group consisting of —OR⁷— and —SR⁷—;
—Y² is a functional group capable of connecting with a reactive polymer;
—R⁶ is selected from the group consisting of —H, alkyl, aryl, heteroaryl, arylalkyl, and heteroarylalkyl; and
—R⁷ is selected from the group consisting of alkylene, phenylene and (OCH₂CH₂)ₚ, with p being an integer ranging from 1 to 1000.

In certain embodiments, —X of formula (PL-1) comprises an activated carbonate such as succinimidyl carbonate, sulfosuccinimidyl carbonate, or nitrophenyl carbonate. In certain embodiments, —X of formula (PL-1) comprises a carbonyl halide such as O(C═0)C₁ or O(C═0)F. In certain embodiments, —X of formula (PL-1) has the formula (PL-2). In certain embodiments, —X of formula (PL-1) is OR⁷ or SR⁷, wherein R⁷ is optionally substituted alkylene, optionally substituted phenylene or (OCH₂CH₂)ₚ, wherein p is 1 to 1000.

In certain embodiments, p of formula (PL-2) is an integer ranging from 1 to 100. In certain embodiments, p of formula (PL-2) is an integer ranging from 1 to 10.

In certain embodiments, —Y¹ of formula (PL-1) and —Y² of formula (PL-2) independently comprise N₃, NH₂, NH—CO₂^{t}Bu, SH, S^{t}Bu, maleimide, CO₂H, CO₂^{t}Bu, 1,3-diene, cyclopentadiene, furan, alkyne, cyclooctyne, acrylate or acrylamide, wherein ^{t}Bu is tert-butyl, and wherein when one of —Y¹ or —Y² comprises N₃ the other does not comprise alkyne or cyclooctyne; when one of —Y¹ or —Y² comprises SH the other does not comprise maleimide, acrylate or acrylamide; when one of —Y¹ or —Y² comprises NH₂ the other does not comprise CO₂H; and when one of —Y¹ or —Y² comprises 1,3-diene or cyclopentadiene the other does not comprise furan.

In certain embodiments, the cleavable crosslinker compound is of formula (PL-3):

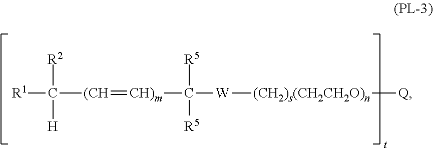

wherein
m is 0 or 1;
n is an integer selected from 1 to 1000;
s is 0, 1 or 2;
t is selected from the group consisting of 2, 4, 8, 16 and 32;
—W— is selected from the group consisting of —O(C═O)O—, —O(C═O)NH—, —O(C═O)S—, —O(C═O)NR⁶CH₂O—and —O(C═O)NR⁶S—;
-Q is a core group having a valency=t; which connects the multiple arms of the cleavable crosslinking compound;
wherein t is an integer selected from 2, 4, 8, 16 and 32; and
wherein —R¹, —R² and —R⁵ are defined as in formula (PL-1).

In certain embodiments, t of formula (PL-3) is 2. In certain embodiments, t of formula (PL-3) is 4. In certain embodiments, t of formula (PL-3) is 8. In certain embodiments, t of formula (PL-3) is 16. In certain embodiments, t of formula (PL-3) is 32.

In certain embodiments, -Q of formula (PL-3) has a structure selected from the group consisting of:

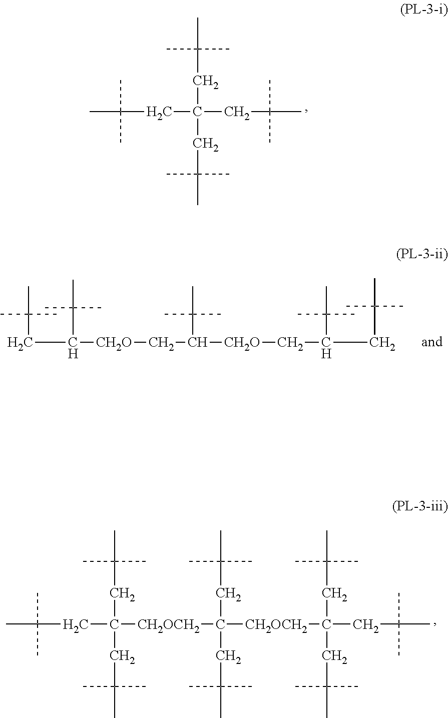

wherein the dashed lines indicate attachment to the remainder of the cleavable crosslinker compound.

In certain embodiments, -Q of formula (PL-3) has the structure of (PL-3-i). In certain embodiments, -Q of formula (PL-3) has the structure of (PL-3-ii). In certain embodiments, -Q of formula (PL-3) has the structure of (PL-3-iii).

In certain embodiments, the cleavable crosslinker compound is of formula (PL-3), wherein m is 0, n is approximately 100, s is 0, t is 4, —W— is —O(C═O)NH—, -Q has the structure of (PL-3i), —R² is H, one —R⁵ is —H and the other —R⁵ is (CH₂)₅N₃, and —R¹ is (4-chlorophenyl)SO₂, phenyl substituted with —SO₂, morpholino-SO₂, or —CN.

In certain embodiments, —Y¹ of formula (PL-3) comprises N₃, NH₂, NH—CO₂′Bu, SH, S^{t}Bu, maleimide, CO₂H, CO₂′Bu, 1,3-diene, cyclopentadiene, furan, alkyne, cyclooctyne, acrylate or acrylamide, wherein ^{t}Bu is tert-butyl.

In certain embodiments, each —Y¹ of formula (PL-1) or (PL-3) and —Y² of formula (PL-2) independently comprises N₃, NH₂, NH—CO₂′Bu, SH, S^{t}Bu, maleimide, CO₂H, CO₂^{t}Bu, 1,3-diene, cyclopentadiene, furan, alkyne, cyclooctyne, acrylate or acrylamide.

In certain embodiments, one of —Y¹ and —Y² is azide and the other is a reactive functional group selected from the group consisting of acetylene, cyclooctyne, and maleimide. In certain embodiments, one of —Y¹ and —Y² is thiol and the other is a reactive functional group selected from the group consisting of maleimide, acrylate, acrylamide, vinylsulfone, vinylsulfonamide, and halocarbonyl. In certain embodiments, one of —Y¹ and —Y² is amine and the other is a selective reactive functional group selected from carboxylic acid and activated carboxylic acid. In certain embodiments, one of —Y¹ and —Y² is maleimide and the other is a selective reactive functional group selected from the group consisting of 1,3-diene, cyclopentadiene, and furan.

In certain embodiments, the first and any second polymer is selected from the group consisting of homopolymeric or copolymeric polyethylene glycols, polypropylene glycols, poly(N-vinylpyrrolidone), polymethacrylates, polyphosphazenes, polylactides, polyacrylamides, polyglycolates, polyethylene imines, agaroses, dextrans, gelatins, collagens, polylysines, chitosans, alginates, hyaluronans, pectins and carrageenans that either comprise suitable reactive functionalities or is of formula [Y³—(CH₂)ₛ(CH₂CH₂O)ₙ]ₜQ, wherein —Y³ is a reactive functional group, s is 0, 1 or 2, n is an integer selected from the group ranging from 10 to 1000, -Q is a core group having valency t, and t is an integer selected from the group consisting of 2, 4, 8, 16 and 32.

In certain embodiments, the first polymer comprises a multi-arm polymer. In certain embodiments, the first polymer comprises at least three arms. In certain embodiments, the first polymer comprises at least four arms. In certain embodiments, the first polymer comprises at least five arms. In certain embodiments, the first polymer comprises at least six arms. In certain embodiments, the first polymer comprises at least seven arms. In certain embodiments, the first polymer comprises at least eight arms.

In certain embodiments, the second polymer comprises a multi-arm polymer. In certain embodiments, the second polymer comprises at least three arms. In certain embodiments, the second polymer comprises at least four arms. In certain embodiments, the second polymer comprises at least five arms. In certain embodiments, the second polymer comprises at least six arms. In certain embodiments, the second polymer comprises at least seven arms. In certain embodiments, the second polymer comprises at least eight arms.

In certain embodiments, the first polymer comprises a 2-arm polyethylene glycol polymer. In certain embodiments, the first polymer comprises a 4-arm polyethylene glycol polymer. In certain embodiments, the first polymer comprises an 8-arm polyethylene glycol polymer. In certain embodiments, the first polymer comprises a 16-arm polyethylene glycol polymer. In certain embodiments, the first polymer comprises a 32-arm polyethylene glycol polymer.

In certain embodiments, the second polymer comprises a 2-arm polyethylene glycol polymer. In certain embodiments, the second polymer comprises a 4-arm polyethylene glycol polymer. In certain embodiments, the second polymer comprises an 8-arm polyethylene glycol polymer. In certain embodiments, the second polymer comprises a 16-arm polyethylene glycol polymer. In certain embodiments, the second polymer comprises a 32-arm polyethylene glycol polymer.

In certain embodiments, the first and a second reactive polymer are reacted with said cleavable crosslinker compound, either sequentially or simultaneously.

In certain embodiments, the first and second functional groups are the same.

Only in the context of formulas (PL-1), (PL-2) and (PL-3) the terms used have the following meaning:

The term “a moiety capable of being cleaved by elimination under physiological conditions” refers to a structure comprising a group H—C—(CH═CH)ₘ—C—X′ wherein m is 0 or 1 and X′ is a leaving group, wherein an elimination reaction as described above to remove the elements of HX′ can occur at a rate such that the half-life of the reaction is between 1 and 10,000 hours under physiological conditions of pH and temperature. Preferably, the half-life of the reaction is between 1 and 5,000 hours, and more preferably between 1 and 1,000 hours, under physiological conditions of pH and temperature. By physiological conditions of pH and temperature is meant a pH of between 7 and 8 and a temperature between 30 and 40 degrees centigrade

The term “reactive polymer and reactive oligomer” refers to a polymer or oligomer comprising functional groups that are reactive towards other functional groups, most preferably under mild conditions compatible with the stability requirements of peptides, proteins, and other biomolecules. Suitable functional groups found in reactive polymers include maleimides, thiols or protected thiols, alcohols, acrylates, acrylamides, amines or protected amines, carboxylic acids or protected carboxylic acids, azides, alkynes including cycloalkynes, 1,3-dienes including cyclopentadienes and furans, alpha-halocarbonyls, and N-hydroxysuccinimidyl, N-hydroxysulfosuccinimidyl, or nitrophenyl esters or carbonates.

The term “functional group capable of connecting to a reactive polymer” refers to a functional group that reacts to a corresponding functional group of a reactive polymer to form a covalent bond to the polymer. Suitable functional groups capable of connecting to a reactive polymer include maleimides, thiols or protected thiols, acrylates, acrylamides, amines or protected amines, carboxylic acids or protected carboxylic acids, azides, alkynes including cycloalkynes, 1,3-dienes including cyclopentadienes and furans, alpha-halocarbonyls, and N-hydroxysuccinimidyl, N-hydroxysulfosuccinimidyl, or nitrophenyl esters or carbonates.

The term “substituted” refers to an alkyl, alkenyl, alkynyl, aryl, or heteroaryl group comprising one or more substituent groups in place of one or more hydrogen atoms. Substituent groups may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched, and cyclic; amino, alkylamino, dialkylamino, silyl including alkylsilyl, alkoxysilyl, and arylsilyl; nitro; cyano; carbonyl; carboxylic acid, carboxylic ester, carboxylic amide; aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

The properties of R¹ and R² may be modulated by the optional addition of electron-donating or electron-withdrawing substituents. The term “electron-donating group” refers to a substituent resulting in a decrease in the acidity of the R¹R²CH; electron-donating groups are typically associated with negative Hammett a or Taft σ* constants and are well-known in the art of physical organic chemistry (Hammett constants refer to aryl/heteroaryl substituents, Taft constants refer to substituents on non-aromatic moieties). Examples of suitable electron-donating substituents include lower alkyl, lower alkoxy, lower alkylthio, amino, alkylamino, dialkylamino, and silyl.

The term “electron-withdrawing group” refers to a substituent resulting in an increase in the acidity of the R¹R²CH group; electron-withdrawing groups are typically associated with positive Hammett a or Taft σ* constants and are well-known in the art of physical organic chemistry. Examples of suitable electron-withdrawing substituents include halogen, difluoromethyl, trifluoromethyl, nitro, cyano, C(═O)—R^{x}, wherein —R^{x} is H, lower alkyl, lower alkoxy, or amino, or S(O)ₘR^{Y}, wherein m is 1 or 2 and —R^{Y} is lower alkyl, aryl, or heteroaryl. As is well-known in the art, the electronic influence of a substituent group may depend upon the position of the substituent. For example, an alkoxy substituent on the ortho- or para-position of an aryl ring is electron-donating, and is characterized by a negative Hammett a constant, while an alkoxy substituent on the meta-position of an aryl ring is electron-withdrawing and is characterized by a positive Hammett a constant.

The terms “alkyl”, “alkenyl”, and “alkynyl” include linear, branched or cyclic hydrocarbon groups of 1 to 8 carbons or 1 to 6 carbons or 1 to 4 carbons, wherein alkyl is a saturated hydrocarbon, alkenyl includes one or more carbon-carbon double bonds and alkynyl includes one or more carbon-carbon triple bonds. Unless otherwise specified these contain 1 to 6 carbons.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

The term “halogen” includes fluoro, chloro, bromo and iodo.

The term “maleimide” is a group of the formula

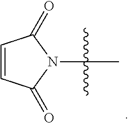 [SMILES: *N1C(=O)C=CC1=O]

In certain embodiments, Z′ is a hydrogel as disclosed in WO 2020/206358 A1. In particular, in certain embodiments, Z′ is a hydrogel produced by a method comprising the steps of
(a) providing a first prepolymer comprising a multi-arm polymer —P², wherein said first prepolymer is of formula (PL-4)

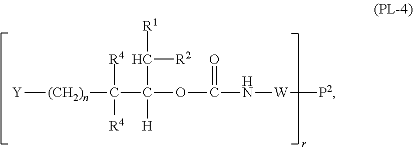

wherein
n is an integer selected from 0, 1, 2, 3, 4, 5 and 6;
r is an integer higher than 2;
—Y is a reactive functional group for connecting said first prepolymer to a second prepolymer;
—R¹ and —R² are independently an electron-withdrawing group, alkyl, or —H, and wherein at least one of —R¹ and —R² is an electron-withdrawing group;
each —R⁴ is independently C₁-C₃ alkyl or the two —R⁴ form together with the carbon atom to which they are attached a 3- to 6-membered ring;
—W— is absent or is

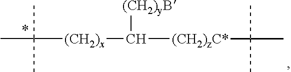

wherein the dashed line marked with the asterisk indicates the attachment to —NH—and the unmarked dashed line indicates the attachment to —P²;
each of x, y, and z is independently an integer selected from 0, 1, 2, 3, 4, 5 and 6;
—B′ is —NH₂, —ONH₂, ketone, aldehyde, —SH, —OH, —CO₂H, carboxamide group, or a group comprising a cyclooctyne or bicyclononyne; and
—C* is carboxamide, thioether, thiosuccinimidyl, triazole, or oxime;
(b) providing the second prepolymer comprising a multi-arm polymer —P¹ wherein each arm is terminated by a reactive functional group —Y″ that reacts with —Y of step (a);
(c) mixing the two prepolymers of steps (a) and (b) under conditions wherein —Y and —Y″ react to form a linkage —Y*—; and optionally
(d) isolating the resulting hydrogel.

Accordingly, Z′ is a hydrogel obtainable from the method described above. In certain embodiments, the hydrogel produced by the preceding method is degradable.

In certain embodiments, —Y and —Y″ react under step (c) to form an insoluble hydrogel matrix comprising crosslinks of formula (PL-4′):

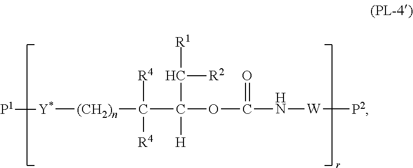

wherein n, r, —P¹, —Y*—, —R⁴, —R¹, —R², —W— and —P² are as defined above.

In certain embodiments, n of formula (PL-4) or (PL-4′) is an integer selected from 1, 2, 3, 4, 5 and 6. In certain embodiments, n of formula (PL-4) or (PL-4′) is an integer selected from 1, 2 and 3. In certain embodiments, n of formula (PL-4) or (PL-4′) is an integer selected from 0, 1, 2 and 3. In certain embodiments, n of formula (PL-4) or (PL-4′) is 1. In certain embodiments, n of formula (PL-4) is 2. In certain embodiments, n of formula (PL-4) or (PL-4′) is 3.

In certain embodiments, the multi-arm —P² of formula (PL-4) or (PL-4′) is an r-armed polymer, wherein r is an integer selected from 2, 3, 4, 5, 6, 7, 8, 9, 10, 11 and 12. In certain embodiments, r of formula (PL-4) or (PL-4′) is an integer selected from 2, 3, 4, 5, 6, 7 and 8. In certain embodiments, r of formula (PL-4) or (PL-4′) is an integer selected from 2, 4, 6 and 8. In certain embodiments, r of formula (PL-4) or (PL-4′) is 2. In certain embodiments, r of formula (PL-4) or (PL-4′) is 4. In certain embodiments, r of formula (PL-4) or (PL-4′) is 6. In certain embodiments, r of formula (PL-4) or (PL-4′) is 8.

In certain embodiments, —P² of formula (PL-4) or (PL-4′) has a molecular weight of at least 1 kDa. In certain embodiments, —P² of formula (PL-4) or (PL-4′) has a molecular weight of 1 to 100 kDa. In certain embodiments, —P² of formula (PL-4) or (PL-4′) has a molecular weight of 1 to 80 kDa. In certain embodiments, —P² of formula (PL-4) or (PL-4′) has a molecular weight of 1 to 60 kDa. In certain embodiments, —P² of formula (PL-4) or (PL-4′) has a molecular weight of 1 to 40 kDa. In certain embodiments, —P² of formula (PL-4) or (PL-4′) has a molecular weight of 1 to 20 kDa. In certain embodiments, —P² of formula (PL-4) or (PL-4′) has a molecular weight of 1 to 10 kDa. In certain embodiments, —P² of formula (PL-4) or (PL-4′) has a molecular weight of 1 to 5 kDa. In certain embodiments, —P² of formula (PL-4) or (PL-4′) has a molecular weight of about 20 kDa. In certain embodiments, —P² of formula (PL-4) or (PL-4′) has a molecular weight of about 40 kDa. In certain embodiments, —P² of formula (PL-4) or (PL-4′) has a molecular weight of about 60 kDa. In certain embodiments, —P² of formula (PL-4) or (PL-4′) has a molecular weight of about 80 kDa.

In certain embodiments, the multi-arm polymer —P¹ of step (b) is an r-armed polymer, wherein r is an integer selected from 2, 3, 4, 5, 6, 7, 8, 9, 10, 11 and 12. In certain embodiments, the multi-arm —P¹ of step (b) is an r-armed polymer, wherein r is an integer selected from 2, 3, 4, 5, 6, 7 and 8. In certain embodiments, the multi-arm —P¹ of step (b) is an r-armed polymer, wherein r is an integer selected from 2, 4, 6 and 8. In certain embodiments, the multi-arm —P¹ of step (b) is an r-armed polymer, wherein r is 2. In certain embodiments, the multi-arm —P¹ of step (b) is an r-armed polymer, wherein r is 4. In certain embodiments, the multi-arm —P¹ of step (b) is an r-armed polymer, wherein r is 6. In certain embodiments, the multi-arm —P¹ of step (b) is an r-armed polymer, wherein r is 8.

In certain embodiments, —P¹ of step (b) has a molecular weight of at least 1 kDa. In certain embodiments, the multi-arm polymer —P¹ of step (b) has a molecular weight of 1 to 100 kDa. In certain embodiments, the multi-arm polymer —P¹ of step (b) has a molecular weight of 1 to 80 kDa. In certain embodiments, the multi-arm polymer —P¹ of step (b) has a molecular weight of 1 to 60 kDa. In certain embodiments, the multi-arm polymer —P¹ of step (b) has a molecular weight of 1 to 40 kDa. In certain embodiments, the multi-arm polymer —P¹ of step (b) has a molecular weight of 1 to 20 kDa. In certain embodiments, the multi-arm polymer —P¹ of step (b) has a molecular weight of 1 to 10 kDa. In certain embodiments, the multi-arm polymer —P¹ of step (b) has a molecular weight of 1 to 5 kDa. In certain embodiments, the multi-arm polymer —P¹ of step (b) has a molecular weight of about 20 kDa. In certain embodiments, the multi-arm polymer —P¹ of step (b) has a molecular weight of about 40 kDa. In certain embodiments, the multi-arm polymer —P¹ of step (b) has a molecular weight of about 60 kDa. In certain embodiments, the multi-arm polymer —P¹ of step (b) has a molecular weight of about 80 kDa.

In certain embodiments, —P¹ of step (b) and —P² of formula (PL-4) or (PL-4′) comprise poly(ethylene glycol) (PEG), poly(ethylene oxide) (PEO), poly(ethylene imine) (PEI), dextrans, hyaluronic acids, or co-polymers thereof. In certain embodiments, —P¹ of step (b) and P² of formula (PL-4) or (PL-4′) are PEG-based polymers. In certain embodiments, —P¹ of step (b) and —P² of formula (PL-4) or (PL-4′) are hyaluronic acid-based polymers.

In certain embodiments, —R¹ and —R² of formula (PL-4) or (PL-4′) are independently electron-withdrawing groups, alkyl, or —H, and wherein at least one of —R¹ and —R² is an electron-withdrawing group.

In certain embodiments, the electron-withdrawing group of —R¹ and —R² of formula (PL-4) or (PL-4′) is —CN, —NO₂, optionally substituted aryl, optionally substituted heteroaryl, optionally substituted alkenyl, optionally substituted alkynyl, —COR³, —SOR³, or —SO₂R³, wherein —R³ is —H, optionally substituted alkyl, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl, or both —R⁸ groups are taken together with the nitrogen to which they are attached to form a heterocyclic ring; or —SR⁹, wherein —R⁹ is optionally substituted alkyl, optionally substituted aryl, optionally substituted arylalkyl, optionally substituted heteroaryl, or optionally substituted heteroarylalkyl.

In certain embodiments, the electron-withdrawing group of —R¹ and —R² of formula (PL-4) or (PL-4′) is —CN. In certain embodiments, the electron-withdrawing group of —R¹ and —R² of formula (PL-4) or (PL-4′) is —NO₂. In certain embodiments, the electron-withdrawing group of —R¹ and —R² of formula (PL-4) or (PL-4′) is optionally substituted aryl containing 6 to 10 carbons. In certain embodiments, the electron-withdrawing group of —R¹ and —R² of formula (PL-4) or (PL-4′) is optionally substituted phenyl, naphthyl, or anthracenyl. In certain embodiments, the electron-withdrawing group of —R¹ and —R² of formula (PL-4) or (PL-4′) is optionally substituted heteroaryl comprising 3 to 7 carbons and containing at least one N, O, or S atom. In certain embodiments, the electron-withdrawing group of —R¹ and —R² of formula (PL-4) or (PL-4′) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments, the electron-withdrawing group of —R¹ and —R² of formula (PL-4) or (PL-4′) is optionally substituted alkenyl containing 2 to 20 carbon atoms. In certain embodiments, the electron-withdrawing group of —R¹ and —R² of formula (PL-4) or (PL-4′) is optionally substituted alkynyl containing 2 to 20 carbon atoms. In certain embodiments, the electron-withdrawing group of —R¹ and —R² of formula (PL-4) or (PL-4′) is —COR³, —SOR³, or —SO₂R³, wherein R³ is —H, optionally substituted alkyl containing 1 to 20 carbon atoms, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl containing 1 to 20 carbon atoms, or both —R⁸ groups are taken together with the nitrogen to which they are attached to form a heterocyclic ring. In certain embodiments, the electron-withdrawing group of —R¹ and —R² of formula (PL-4) or (PL-4′) is —SR⁹, wherein —R⁹ is optionally substituted alkyl containing 1 to 20 carbon atoms, optionally substituted aryl, optionally substituted arylalkyl, optionally substituted heteroaryl, or optionally substituted heteroarylalkyl. In certain embodiments, at least one of —R¹ and —R² is —CN or —SO₂R³.

In certain embodiments, at least one of —R¹ and —R² of formula (PL-4) or (PL-4′) is —CN, —SOR³ or —SO₂R³. In certain embodiments, at least one of —R¹ and —R² of formula (PL-4) or (PL-4′) is —CN or —SO₂R³. In certain embodiments, at least one of —R¹ and —R² of formula (PL-4) or (PL-4′) is —CN or —SO₂R³, wherein —R³ is optionally substituted alkyl, optionally substituted aryl, or —NR⁸₂. In certain embodiments, at least one of —R¹ and —R² of formula (PL-4) or (PL-4′) is —CN, —SO₂N(CH₃)₂, —SO₂CH₃, phenyl substituted with —SO₂, phenyl substituted with —SO₂ and —Cl, —SO₂N(CH₂CH₂)₂O, —SO₂CH(CH₃)₂, —SO₂N(CH₃)(CH₂CH₃), or —SO₂N(CH₂CH₂OCH₃)₂.

In certain embodiments, each —R⁴ of formula (PL-4) or (PL-4′) is independently C₁-C₃ alkyl or taken together may form a 3- to 6-membered ring. In certain embodiments, each —R⁴ of formula (PL-4) or (PL-4′) is independently C₁-C₃ alkyl. In certain embodiments, both —R⁴ of formula (PL-4) or (PL-4′) are methyl.

In certain embodiments, —Y and —Y″ are independently selected from the group consisting of amine, aminooxy, ketone, aldehyde, maleimidyl, thiol, alcohol, azide, 1,2,4,6-tetrazinyl, trans-cyclooctenyl, bicyclononynyl, cyclooctynyl, and protected variants thereof.

In certain embodiments, Y and Y″ may react with each other such as in a selective way. For example, when —Y is amine, —Y″ is carboxylic acid, active ester, or active carbonate to yield a residual connecting functional group —Y*—that is amide or carbamate. As another example, when —Y is azide, —Y″ is alkynyl, bicyclononynyl, or cyclooctynyl to yield a residual connecting functional group —Y*—that is 1,2,3-triazole. As another example, when —Y is NH₂O, —Y″ is ketone or aldehyde to yield a residual connecting functional group —Y*—that is oxime. As another example, when —Y is SH, —Y″ is maleimide or halocarbonyl to yield a residual connecting functional group —Y*—that is thiosuccinimidyl or thioether. Similarly, these roles of —Y and —Y″ can be reversed to yield —Y*—of opposing orientation.

In certain embodiments, —Y*—comprises an amide, oxime, 1,2,3-triazole, thioether, thiosuccinimide, or ether. In certain embodiments, —Y*—is -L²-.

These conjugation reactions may be performed under conditions known in the art, for example when —Y is azide and —Y″ is cyclooctyne the conjugation occurs in any solvent wherein both components show adequate solubility, although it is known that aqueous solutions show more favorable reaction rates. When mixed in an appropriate solvent, typically an aqueous buffer at a pH of 2 to 7 when —Y and —Y″ are azide/cyclooctyne, or at a pH of 6 to 9 when —Y and —Y″ are an activated ester and an amine, the —Y and —Y″ groups react to form an insoluble hydrogel matrix comprising crosslinks of formula (PL-4′). This process may be carried out in bulk phase, or under conditions of emulsification in a mixed organic/aqueous system so as to form microparticle suspensions such as microspheres that are suitable for injection.

Only in the context of formulas (PL-4) and (PL-4′) the terms used have the following meaning: The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n- octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments, alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro or iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments, a heterocyclic ring or heterocyclyl is non-aromatic. In certain embodiments, a heterocyclic ring or heterocyclyl is aromatic.

The term “optionally substituted” refers to a group that may be unsubstituted or substituted by one or more (e.g., 1, 2, 3, 4 or 5) of the substituents which may be the same or different.

Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each —R^{cc} is independently alkyl, alkenyl, alkynyl, halogen, heterocyclyl, heteroaryl, aryl, —CN, or —NO₂.

In certain embodiments the first PTH compound is a compound of formula (I)

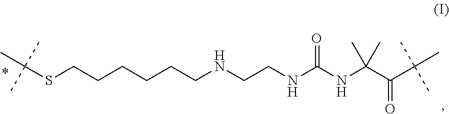

wherein
the unmarked dashed line indicates the attachment to the nitrogen of the N-terminal amine group of a PTH moiety of SEQ ID NO:51; and
the dashed line marked with the asterisk indicates attachment to a moiety

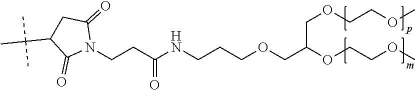

wherein
m and p are independently an integer ranging from approx. 400 to 500.

In certain embodiments m and p of formula (I) are independently an integer ranging from 400 to 500. In certain embodiment m and p of formula (I) are independently an integer ranging from approx. 450 to 500. In certain embodiment m and p of formula (I) are independently an integer ranging from 450 to 500.

The compound of formula (I) is also known as palopegteriparatide, TransCon PTH or ACP-014.

It is understood that the nitrogen of the N-terminal amine group of the PTH moiety of formula (I) and the carbonyl (—(C═O)—) group to the left of the unmarked dashed line form an amide bond.

The PTH compound of formula (I) releases PTH of SEQ ID NO:51, PTH 1-34, meaning that its active PTH is PTH 1-34.

In certain embodiments the first PTH compound is a compound of formula (I) and the first average interval is approx. one day. In certain embodiments the first PTH compound is a compound of formula (I) and the first average interval is one day. In certain embodiments the first PTH compound is a compound of formula (I), the first average interval is approx. one day and administration is via subcutaneous injection. In certain embodiments the first PTH compound is a compound of formula (I), the first average interval is one day and administration is via subcutaneous injection. In certain embodiments the first PTH compound is a compound of formula (I), the first average interval is approx. one day and administration is via subcutaneous injection with a pen injector. In certain embodiments the first PTH compound is a compound of formula (I), the first average interval is one day and administration is via subcutaneous injection with a pen injector. In certain embodiments the first PTH compound is a compound of formula (I) with m and p independently being an integer ranging from approx. 450 to 500, the first average interval is one day and administration is via subcutaneous injection with a pen injector. In certain embodiments the first PTH compound is a compound of formula (I) with m and p independently being an integer ranging from 450 to 500, the first average interval is one day and administration is via subcutaneous injection with a pen injector.

In certain embodiments the first PTH compound is a compound of formula (I) and the first interval is approx. one day. In certain embodiments the first PTH compound is a compound of formula (I) and the first interval is one day. In certain embodiments the first PTH compound is a compound of formula (I), the first interval is approx. one day and administration is via subcutaneous injection. In certain embodiments the first PTH compound is a compound of formula (I), the first interval is one day and administration is via subcutaneous injection. In certain embodiments the first PTH compound is a compound of formula (I), the first interval is approx. one day and administration is via subcutaneous injection with a pen injector. In certain embodiments the first PTH compound is a compound of formula (I), the first interval is one day and administration is via subcutaneous injection with a pen injector. In certain embodiments the first PTH compound is a compound of formula (I) with m and p independently being an integer ranging from approx. 450 to 500, the first interval is one day and administration is via subcutaneous injection with a pen injector. In certain embodiments the first PTH compound is a compound of formula (I) with m and p independently being an integer ranging from 450 to 500, the first interval is one day and administration is via subcutaneous injection with a pen injector.

In certain embodiments the first PTH compound is PTH 1-34 (SEQ ID NO:51), also known as Forteo®. The active PTH of Forteo is PTH 1-34.

In certain embodiments the first PTH compound is PTH 1-34 and the first average interval is approx. 8 hours. In certain embodiments the first PTH compound is PTH 1-34 and the first average interval is 8 hours. In certain embodiments the first PTH compound is PTH 1-34, the first average interval is approx. 8 hours and administration is via subcutaneous injection. In certain embodiments the first PTH compound is PTH 1-34, the first average interval is 8 hours and administration is via subcutaneous injection. In certain embodiments the first PTH compound is PTH 1-34, the first average interval is approx. 8 hours and administration is via subcutaneous injection with a pen injector. In certain embodiments the first PTH compound is PTH 1-34, the first average interval is 8 hours and administration is via subcutaneous injection with a pen injector.

In certain embodiments the first PTH compound is PTH 1-34 and the first interval is approx. 8 hours. In certain embodiments the first PTH compound is PTH 1-34 and the first interval is 8 hours. In certain embodiments the first PTH compound is PTH 1-34, the first interval is approx. 8 hours and administration is via subcutaneous injection. In certain embodiments the first PTH compound is PTH 1-34, the first interval is 8 hours and administration is via subcutaneous injection. In certain embodiments the first PTH compound is PTH 1-34, the first interval is approx. 8 hours and administration is via subcutaneous injection with a pen injector. In certain embodiments the first PTH compound is PTH 1-34, the first interval is 8 hours and administration is via subcutaneous injection with a pen injector.

In certain embodiments the first PTH compound is PTH 1-34 and the first average interval is approx. 12 hours. In certain embodiments the first PTH compound is PTH 1-34 and the first average interval is 12 hours. In certain embodiments the first PTH compound is PTH 1-34, the first average interval is approx. 12 hours and administration is via subcutaneous injection. In certain embodiments the first PTH compound is PTH 1-34, the first average interval is 12 hours and administration is via subcutaneous injection. In certain embodiments the first PTH compound is PTH 1-34, the first average interval is approx. 12 hours and administration is via subcutaneous injection with a pen injector. In certain embodiments the first PTH compound is PTH 1-34, the first average interval is 12 hours and administration is via subcutaneous injection with a pen injector.

In certain embodiments the first PTH compound is PTH 1-34 and the first interval is approx. 12 hours. In certain embodiments the first PTH compound is PTH 1-34 and the first interval is 12 hours. In certain embodiments the first PTH compound is PTH 1-34, the first interval is approx. 12 hours and administration is via subcutaneous injection. In certain embodiments the first PTH compound is PTH 1-34, the first interval is 12 hours and administration is via subcutaneous injection. In certain embodiments the first PTH compound is PTH 1-34, the first interval is approx. 12 hours and administration is via subcutaneous injection with a pen injector. In certain embodiments the first PTH compound is PTH 1-34, the first interval is 12 hours and administration is via subcutaneous injection with a pen injector.

In certain embodiments the first PTH compound is PTH 1-34 and the first average interval is approx. one day. In certain embodiments the first PTH compound is PTH 1-34 and the first average interval is one day. In certain embodiments the first PTH compound is PTH 1-34, the first average interval is approx. one day and administration is via subcutaneous injection. In certain embodiments the first PTH compound is PTH 1-34, the first average interval is one day and administration is via subcutaneous injection. In certain embodiments the first PTH compound is PTH 1-34, the first average interval is approx. one day and administration is via subcutaneous injection with a pen injector. In certain embodiments the first PTH compound is PTH 1-34, the first average interval is one day and administration is via subcutaneous injection with a pen injector.

In certain embodiments the first PTH compound is PTH 1-34 and the first interval is approx. one day. In certain embodiments the first PTH compound is PTH 1-34 and the first interval is one day. In certain embodiments the first PTH compound is PTH 1-34, the first interval is approx. one day and administration is via subcutaneous injection. In certain embodiments the first PTH compound is PTH 1-34, the first interval is one day and administration is via subcutaneous injection. In certain embodiments the first PTH compound is PTH 1-34, the first interval is approx. one day and administration is via subcutaneous injection with a pen injector.

In certain embodiments the first PTH compound is PTH 1-34, the first interval is one day and administration is via subcutaneous injection with a pen injector.

In certain embodiments the first PTH compound is PTH 1-84 (SEQ ID NO:1), which is also known as Natpara® or Natpar®. The active PTH of Natpara/Natpar is PTH 1-84.

In certain embodiments the first PTH compound is PTH 1-84 and the first average interval is approx. one day. In certain embodiments the first PTH compound is PTH 1-84 and the first average interval is one day. In certain embodiments the first PTH compound is PTH 1-84, the first average interval is approx. one day and administration is via subcutaneous injection. In certain embodiments the first PTH compound is PTH 1-84, the first average interval is one day and administration is via subcutaneous injection. In certain embodiments the first PTH compound is PTH 1-84, the first average interval is approx. one day and administration is via subcutaneous injection with a pen injector. In certain embodiments the first PTH compound is PTH 1-84, the first average interval is one day and administration is via subcutaneous injection with a pen injector.

In certain embodiments the first PTH compound is PTH 1-84 and the first interval is approx. one day. In certain embodiments the first PTH compound is PTH 1-84 and the first interval is one day. In certain embodiments the first PTH compound is PTH 1-84, the first interval is approx. one day and administration is via subcutaneous injection. In certain embodiments the first PTH compound is PTH 1-84, the first interval is one day and administration is via subcutaneous injection. In certain embodiments the first PTH compound is PTH 1-84, the first interval is approx. one day and administration is via subcutaneous injection with a pen injector.

In certain embodiments the first PTH compound is PTH 1-84, the first interval is one day and administration is via subcutaneous injection with a pen injector.

In certain embodiments the first PTH compound is a compound having the sequence of SEQ ID NO:122, which is also known as AZP-3601:

(SEQ ID NO: 122)
AVAEIQLMHQRAKWIQDARRRAFLHKLIAEIHTAEI

The active PTH of AZP-3601 is the protein of SEQ ID NO:122.

In certain embodiments the first PTH compound is a compound having the sequence of SEQ ID NO:122 and the first average interval is approx. one day. In certain embodiments the first PTH compound is a compound having the sequence of SEQ ID NO:122 and the first average interval is one day. In certain embodiments the first PTH compound is a compound having the sequence of SEQ ID NO:122, the first average interval is approx. one day and administration is via subcutaneous injection. In certain embodiments the first PTH compound is a compound having the sequence of SEQ ID NO:122, the first average interval is one day and administration is via subcutaneous injection. In certain embodiments the first PTH compound is a compound having the sequence of SEQ ID NO:122, the first average interval is approx. one day and administration is via subcutaneous injection with a pen injector. In certain embodiments the first PTH compound is a compound having the sequence of SEQ ID NO:122, the first average interval is one day and administration is via subcutaneous injection with a pen injector.

In certain embodiments the first PTH compound is a compound having the sequence of SEQ ID NO:122 and the first interval is approx. one day. In certain embodiments the first PTH compound is a compound having the sequence of SEQ ID NO:122 and the first interval is one day. In certain embodiments the first PTH compound is a compound having the sequence of SEQ ID NO:122, the first interval is approx. one day and administration is via subcutaneous injection. In certain embodiments the first PTH compound is a compound having the sequence of SEQ ID NO:122, the first interval is one day and administration is via subcutaneous injection. In certain embodiments the first PTH compound is a compound having the sequence of SEQ ID NO:122, the first interval is approx. one day and administration is via subcutaneous injection with a pen injector. In certain embodiments the first PTH compound is a compound having the sequence of SEQ ID NO:122, the first interval is one day and administration is via subcutaneous injection with a pen injector.

In certain embodiments step (a) is administering to a patient having chronic hypoparathyroidism for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a patient having chronic hypoparathyroidism for a first treatment period ranging from one week to 5 years the compound of formula (I) with m and p independently being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a patient having chronic hypoparathyroidism for a first treatment period ranging from one week to 5 years the compound of formula (I) with m and p independently being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism for a first treatment period ranging from one week to 5 years the compound of formula (I) with m and p being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism for a first treatment period ranging from one week to 5 years the compound of formula (I) with m and p being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism for a first treatment period ranging from one week to 5 years the compound of formula (I) with m and p being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism for a first treatment period ranging from one week to 5 years the compound of formula (I) with m and p being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to surgery, a genetic cause, immune system-related damage of the parathyroid glands or having idiopathic hypoparathyroidism for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to surgery for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to surgery for a first treatment period ranging from one week to 5 years the compound of formula (I) with m and p being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to surgery for a first treatment period ranging from one week to 5 years the compound of formula (I) with m and p being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to surgery for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to surgery for a first treatment period ranging from one week to 5 years the compound of formula (I) with m and p independently being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to surgery for a first treatment period ranging from one week to 5 years the compound of formula (I) with m and p independently being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to a genetic cause for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to a genetic cause for a first treatment period ranging from one week to 5 years the compound of formula (I) with m and p independently being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to a genetic cause for a first treatment period ranging from one week to 5 years the compound of formula (I) with m and p independently being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to a genetic cause for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to a genetic cause for a first treatment period ranging from one week to 5 years the compound of formula (I) with m and p independently being an integer ranging from approx. 450 to approx. 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to a genetic cause for a first treatment period ranging from one week to 5 years the compound of formula (I) with m and p independently being an integer ranging from 450 to approx. 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to immune-related damage of the parathyroid glands for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to immune-related damage of the parathyroid glands for a first treatment period ranging from one week to 5 years the compound of formula (I) with m and p independently being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to immune-related damage of the parathyroid glands for a first treatment period ranging from one week to 5 years the compound of formula (I) with m and p independently being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to immune-related damage of the parathyroid glands for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to immune-related damage of the parathyroid glands for a first treatment period ranging from one week to 5 years the compound of formula (I) with m and p being independently being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to immune-related damage of the parathyroid glands for a first treatment period ranging from one week to 5 years the compound of formula (I) with m and p being independently being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to surgery, a genetic cause, immune system-related damage of the parathyroid glands or having idiopathic hypoparathyroidism for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to surgery for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to surgery for a first treatment period ranging from one week to 5 years the compound of formula (I) with m and p independently being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to surgery for a first treatment period ranging from one week to 5 years the compound of formula (I) with m and p independently being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to surgery for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to surgery for a first treatment period ranging from one week to 5 years the compound of formula (I) with m and p independently being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to surgery for a first treatment period ranging from one week to 5 years the compound of formula (I) with m and p independently being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to a genetic cause for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to a genetic cause for a first treatment period ranging from one week to 5 years the compound of formula (I) with m and p independently being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to a genetic cause for a first treatment period ranging from one week to 5 years the compound of formula (I) with m and p independently being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to a genetic cause for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to a genetic cause for a first treatment period ranging from one week to 5 years the compound of formula (I) with m and p independently being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to a genetic cause for a first treatment period ranging from one week to 5 years the compound of formula (I) with m and p independently being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to immune-related damage of the parathyroid glands for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to immune-related damage of the parathyroid glands for a first treatment period ranging from one week to 5 years the compound of formula (I) with m and p independently being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to immune-related damage of the parathyroid glands for a first treatment period ranging from one week to 5 years the compound of formula (I) with m and p independently being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to immune-related damage of the parathyroid glands for a first treatment period ranging from one week to 5 years the compound of formula (I) via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to immune-related damage of the parathyroid glands for a first treatment period ranging from one week to 5 years the compound of formula (I) with m and p independently being an integer ranging from approx. 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to immune-related damage of the parathyroid glands for a first treatment period ranging from one week to 5 years the compound of formula (I) with m and p independently being an integer ranging from 450 to 500 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a patient having chronic hypoparathyroidism for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to surgery for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to surgery for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to a genetic cause for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to a genetic cause for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to immune-related damage of the parathyroid glands for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to immune-related damage of the parathyroid glands for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to surgery for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to surgery for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to a genetic cause for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to a genetic cause for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to immune-related damage of the parathyroid glands for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to immune-related damage of the parathyroid glands for a first treatment period ranging from one week to 5 years PTH 1-34 via subcutaneous injection with a first interval between administrations of eight hours, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a patient having chronic hypoparathyroidism for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to surgery for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to surgery for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to a genetic cause for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to a genetic cause for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to immune-related damage of the parathyroid glands for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to immune-related damage of the parathyroid glands for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to surgery for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to surgery for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to a genetic cause for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to a genetic cause for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to immune-related damage of the parathyroid glands for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to immune-related damage of the parathyroid glands for a first treatment period ranging from one week to 5 years PTH 1-84 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a patient having chronic hypoparathyroidism for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to surgery for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to surgery for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to a genetic cause for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to a genetic cause for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to immune-related damage of the parathyroid glands for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to a pediatric patient having chronic hypoparathyroidism due to immune-related damage of the parathyroid glands for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement.

In certain embodiments step (a) is administering to an adult patient having chronic hypoparathyroidism due to surgery for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to surgery for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to a genetic cause for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to a genetic cause for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to an adult patient having chronic hypoparathyroidism due to immune-related damage of the parathyroid glands for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments step (a) is administering to a pediatric patient having chronic hypoparathyroidism due to immune-related damage of the parathyroid glands for a first treatment period ranging from one week to 5 years a compound comprising the sequence of SEQ ID NO:122 via subcutaneous injection with a first interval between administrations of one day, adjusting the dose if needed, to determine for the patient a dose at which the patient's serum calcium level remains with normal range in the absence of active vitamin D and calcium supplement, and wherein the patient has been a stable patient with no dose adjustments for the last two, three, four, five, six, seven, eight, nine, ten, eleven, twelve, thirteen or fourteen intervals between administrations of the first treatment period before administration of the first dose of the second PTH compound.

In certain embodiments the dose of the first PTH compound is increased between at least two consecutive administrations in response to hypocalcemia. Upon increasing the dose, the patient may remain on said increased dose for one or more consecutive administrations or may remain on the increased dose for all administrations in the first treatment period. In certain embodiments the patient may after one or a few, such as two, three, four or five increased doses return to the dose prior to the increase, i.e., to the lower dose. In certain embodiments the dose of the first PTH compound is further increased between at least two consecutive administrations in response to persistent hypocalcemia and, if necessary, further dose increases may be made.

In certain embodiments the dose of the first PTH compound is decreased between at least two consecutive administrations responsive to hypercalcemia. Upon decreasing the dose, the patient may remain on said decreased dose for one or more consecutive administrations or may remain on the decreased dose for all administrations in the first treatment period. In certain embodiments the patient may after one or a few, such as two, three, four or five decreased doses return to the dose prior to the decrease, i.e., to the higher dose. In certain embodiments the dose of the first PTH compound is further decreased between at least two consecutive administrations in response to persistent hypercalcemia and, if necessary, further dose decreases may be made.

It is understood that step (a) may comprise any combination of increases and decreases of the dos of the first PTH compound.

In certain embodiments the starting dose for the compound of formula (I) ranges from 3 to 5 nmol per day. In certain embodiments the starting dose for the compound of formula (I) ranges from 3.5 to 4.5 nmol per day. In certain embodiments the starting dose for the compound of formula (I) is 4.4±0.3 nmol per day. In certain embodiments the starting dose for the compound of formula (I) is 4.4±0.2 nmol per day. In certain embodiments the starting dose for the compound of formula (I) is 4.4±0.1 nmol per day.

In certain embodiments the starting dose for the compound of formula (I) ranges from 12 to 24 μg PTH 1-34 per day. In certain embodiments the starting dose for the compound of formula (I) ranges from 15 to 21 μg PTH 1-34 per day. In certain embodiments the starting dose for the compound of formula (I) is 18±3 μg PTH 1-34 per day. In certain embodiments the starting dose for the compound of formula (I) is 18 μg PTH 1-34 per day.

In the context of the compound of formula (I) a dose is generally given as μg PTH 1-34, meaning that for the calculation of the dose only the weight of the PTH moiety, i.e., PTH 1-34, is considered, but not the weight of the remainder of the compound, i.e., the moiety to the left of the unmarked dashed line in formula (I).

In certain embodiments the first PTH compound is the compound of formula (I) and is administered to the patient in a pharmaceutical composition. In certain embodiments 1 ml of such pharmaceutical composition comprises 3456 μg of the compound of formula (I), which corresponds to 300 μg of PTH 1-34, 1.18 mg succinic acid, 41.7 mg mannitol, 2.5 mg metacresol, 0.13 mg sodium hydroxide and water for injection and has a pH of 3.7 to 4.3. In certain embodiments 1 ml of such pharmaceutical composition comprises 3456 μg of the PTH compound of formula (I), which corresponds to 300 μg of PTH 1-34, 1.18 mg succinic acid, 41.7 mg mannitol, 2.5 mg metacresol, 0.13 mg sodium hydroxide and water for injection and has a pH of 3.7 to 4.3.

In certain embodiments the number of presentations of such pharmaceutical composition ranges from 2 to 8, each presentation comprising a different dose of the PTH compound of formula (I). In certain embodiments the number of presentations of the pharmaceutical composition ranges from 2 to 6, each presentation comprising a different dose of the PTH compound of formula (I). In certain embodiments the number of presentations of the pharmaceutical composition ranges from 2 to 4, each presentation comprising a different dose of the PTH compound of formula (I). In certain embodiments the pharmaceutical composition is provided in 3 presentations, each presentation comprising a different dose of the PTH compound of formula (I). In certain embodiments each presentation is a prefilled pen.

In certain embodiments the pharmaceutical composition is provided in 3 presentations, wherein the first presentation is a prefilled pen comprising 168 μg PTH 1-34/0.56 mL, the second presentation is a prefilled pen comprising 294 μg PTH 1-34/0.98 mL and the third presentation is a prefilled pen comprising 420 μg PTH 1-34/1.4 ml, wherein the PTH 1-34 is provided in the form of the PTH compound of formula (I).

The first of these three presentations may be used to administer doses of 6, 9 or 12 μg PTH 1-34/day, the second presentation may be used to administer doses of 15, 18 or 21 μg PTH 1-34/day and the third presentation may be used to administer doses of 24, 27 or 30 μg PTH 1-34/day. Doses higher than 30 and up to and including 60 μg PTH 1-34/day may be administered as two sequential administrations. For example, a dose of 33 μg PTH 1-34/day may be administered as a combination of a dose of 15 μg PTH 1-34/day+a dose of 18 μg PTH 1-34/day; a dose of 36 μg PTH 1-34/day may be administered as a combination of a dose of 18 μg PTH 1-34/day+a dose of 18 μg PTH 1-34/day; a dose of 39 μg PTH 1-34/day may be administered as a combination of a dose of 18 μg PTH 1-34/day+a dose of 21 μg PTH 1-34/day; a dose of 42 μg PTH 1-34/day may be administered as a combination of a dose of 21 μg PTH 1-34/day+a dose of 21 μg PTH 1-34/day; a dose of 45 μg PTH 1-34/day may be administered as a combination of a dose of 21 μg PTH 1-34/day+a dose of 24 μg PTH 1-34/day; a dose of 48 μg PTH 1-34/day may be administered as a combination of a dose of 24 μg PTH 1-34/day+a dose of 24 μg PTH 1-34/day; a dose of 51 μg PTH 1-34/day may be administered as a combination of a dose of 24 μg PTH 1-34/day+a dose of 27 μg PTH 1-34/day; a dose of 54 μg PTH 1-34/day may be administered as a combination of a dose of 27 μg PTH 1-34/day+a dose of 27 μg PTH 1-34/day; a dose of 57 μg PTH 1-34/day may be administered as a combination of a dose of 27 μg PTH 1-34/day+a dose of 30 μg PTH 1-34/day; and a dose of 60 μg PTH 1-34/day may be administered as a combination of a dose of 30 μg PTH 1-34/day+a dose of 30 μg PTH 1-34/day, wherein the PTH 1-34 is provided by the compound of formula (I).

In a patient having hypoparathyroidism the treatment of step (a) may be initiated by
(i-a) confirming that the serum 25(OH) vitamin D of the patient is within the normal range within two weeks before the first dose of the compound of formula (I) is administered to the patient and serum calcium is >7.8 mg/dL at the initiation of treatment;
(i-b) if the patient is taking active vitamin D at the time treatment with the compound of formula (I) is initiated:
   (i-b-i) maintaining the same dose of calcium supplements and discontinue active vitamin D on the same day as the first dose of the compound of formula (I) is administered, if serum calcium is >8.3 mg/dL; or
   (i-b-ii) reducing the dose of active vitamin D by >50% on the same day as the first dose of the compound of formula (I) is administered and maintaining the same dose of calcium supplements, if serum calcium is <8.3 mg/dL; or
if the patient is not taking active vitamin D at the time treatment with the compound of formula (I) is initiated:
   (i-b-iii) decreasing calcium supplements by at least 1500 mg on the same day as the first dose of the compound of formula (I) is administered; and
(i-c) optionally continuing dietary calcium supplements at doses of <600 mg/day as indicated to meet dietary calcium requirements.

In certain embodiments serum 25(OH) vitamin D is within the normal range if its concentration is between 20 and 80 ng/ml.

If the patient is not taking active vitamin D and if calcium supplement doses of 1500 mg calcium/day are taken, calcium supplement doses are discontinued entirely in step (i-b-iii).

In certain embodiments step (a) may in addition to steps (i-a) to (i-c) further comprise the steps of
(i-d) administering a starting dose of 18 μg PTH 1-34/day in the form of the compound of formula (I), followed by daily administrations of the same dose;
(i-e) measuring serum calcium within 7 to 14 days of the first administration of the compound of formula (I); and
(i-f) adjusting the dose of the compound of formula (I), active vitamin D and/or calcium supplement.

In certain embodiments the compound of formula (I) is administered in step (i-d) as a pharmaceutical composition, of which each ml comprises 3456 μg of the compound of formula (I), which corresponds to 300 μg of PTH 1-34, 1.18 mg succinic acid, 41.7 mg mannitol, 2.5 mg metacresol, 0.13 mg sodium hydroxide, and water for injection. In certain embodiments the pharmaceutical composition has a pH of 3.7 to 4.3.

Adjusting the dose of the compound of formula (I), active vitamin D and/or calcium supplement in step (i-f) is performed based on the serum calcium levels from step (i-e).

In certain embodiments adjustments in the dose of the compound of formula (I), active vitamin D and/or calcium supplement of step (i-f) are performed as follows:
(i-f-i) if serum calcium is <8.3 mg/dL:
if >7 days have passed since treatment with the compound of formula (I) was started or the dose of the compound of formula (I) was changed, the same calcium supplement and active vitamin D doses are continued, and the dose of the compound of formula (I) is increased by 3 μg; or
if fewer than 7 days have passed since treatment with the compound of formula (I) has started or the dose of the compound of formula (I) was changed, calcium supplements and/or active vitamin D are increased toward prior doses based on physician's clinical judgement and the same dose of the compound of formula (I) is continued;
(i-f-ii) if serum calcium ranges from 8.3 to 10.6 mg/dL:
   if ≥7 days have passed since treatment with the compound of formula (I) was started or since the dose of the compound of formula (I) was changed and the patient is still taking active vitamin D, active vitamin D is discontinued, and the dose of the compound of formula (I) is increased by 3 μg;
   if ≥7 days have passed since treatment with the compound of formula (I) was started or since the dose of the compound of formula (I) was changed and the patient is no longer taking active vitamin D, but is taking calcium supplements, wherein the calcium supplement is ≥1500 mg/day, the calcium supplement is decreased by ≥1500 mg and the dose of the compound of formula (I) is increased by 3 μg;
   if ≥7 days have passed since treatment with the compound of formula (I) was started or since the dose of the compound of formula (I) was changed and the patient is no longer taking active vitamin D, but is taking calcium supplements, wherein the calcium supplement is less than 1500 mg/day, calcium supplements are discontinued, and the dose of the compound of formula (I) is increased by 3 μg;
   if ≥7 days have passed since treatment with the compound of formula (I) was started or since the dose of the compound of formula (I) was changed and the patient is no longer taking active vitamin D and is no longer taking calcium supplements, the same dose of the compound of formula (I) is continued; or
   if fewer than 7 days have passed since treatment with the compound of formula (I) was started or since the dose of the compound of formula (I) was changed, the same dose of the compound of formula (I), of the calcium supplement and active vitamin D is continued;
(i-f-iii) if serum calcium ranges from 10.7 to 11.9 mg/dL:
   if the patient is still taking active vitamin D, active vitamin D is discontinued and the same doses of the compound of formula (I) and calcium supplement are continued;
   if the patient is not taking active vitamin D but takes calcium supplements at a dose of ≥1500 mg/day, calcium supplements are decreased by >1500 mg and the same dose of the compound of formula (I) is continued;
   if the patient is not taking active vitamin D but takes calcium supplements, which calcium supplements are less than 1500 mg per day, calcium supplements are discontinued, and the same dose of the compound of formula (I) is continued; or
   if the patient is not taking active vitamin D and does not take calcium supplements, the dose of the compound of formula (I) is decreased by 3 μg; or
(i-f-iv) if serum calcium is >12 mg/dL:
   withhold the compound of formula (I) for 2 to 3 days, recheck serum calcium and
   if subsequent serum calcium is <12 mg/dL, resume titration of the compound of formula (I), active vitamin D and calcium supplements as described in steps (i-f-i) to (i-f-iii) using the most recent serum calcium value obtained; and
   if serum calcium remains ≥12 mg/dL, withhold the compound of formula (I) for an additional 2 to 3 days, recheck serum calcium and proceed as described in the previous step.

In certain embodiments the dosage range of the compound of formula (I) administered to the patient ranges from and includes 6 to 60 μg PTH 1-34/day, which doses may be provided in prefilled pens of 168 μg PTH 1-34/0.56 mL (delivering doses of 6, 9 or 12 μg PTH 1-34); 294 μg PTH 1-34/0.98 mL (delivering doses of 15, 18, or 21 μg PTH 1-34); and 420 μg PTH 1-34/1.4 ml (delivering doses of 24, 27, or 30 μg PTH 1-34).

In certain embodiments the compound of formula (I) or the pharmaceutical composition comprising the compound of formula (I) is inspected visually for particulate matter and discoloration prior to administration.

In certain embodiments dose adjustments of the first PTH compound of formula (I), active vitamin D and calcium supplement are made on the same day. After a dose change in the compound of first PTH compound of formula (I), active vitamin D or calcium supplements, serum calcium is in certain embodiments measured within 7 to 14 days and the patient may be monitored for clinical symptoms of hypocalcemia or hypercalcemia and doses of the first PTH compound of formula (I), active vitamin D and/or calcium supplements may be adjusted as described above.

The dose of the first PTH compound of formula (I) may be increased as described above in increments of 3 μg if at least 7 days have elapsed since a prior dose change of the first PTH compound of formula (I). In certain embodiments the dose of the first PTH compound of formula (I) administered to the patient is no more often than every 7 days adjusted. The dose of the first PTH compound of formula (I) may be reduced no more often than every 3 days in 3 μg increments in response to hypercalcemia as described above.

The maintenance dose should be the dose of the first PTH compound of formula (I) that achieves serum calcium within the normal range, without the need for active vitamin D and calcium supplement. Optionally, calcium supplementation sufficient to meet dietary requirements may be continued. Serum calcium may be measured per standard of care once a maintenance dose is achieved.

In certain embodiments step (a) may in addition to steps (i-a) to (i-f) further comprise the steps of
(i-g) administration of a daily maintenance dose; and
(i-h) measuring serum calcium per standard of care.

Optionally, step (i-h) is followed by repeating steps (i-f) to (i- h), if serum calcium levels are not within the normal range, such as within a range from 8.3 to 10.6 mg/dL.

If a dose of the first PTH compound of formula (I) is missed by less than 12 hours, it may be taken as soon as possible. If a dose is missed by more than 12 hours, it may be skipped, and the next dose is then taken as scheduled. If administration of 3 or more consecutive doses is missed, it is recommended to monitor for signs and symptoms of hypocalcemia and to consider measuring serum calcium. If indicated, treatment with active vitamin D and calcium supplement may be resumed. In certain embodiments administration of the first PTH compound of formula (I) is resumed at the prescribed dose as soon as possible after an interruption, which prescribed dose may be the maintenance dose or not. When resuming treatment after an interruption, serum calcium should be measured and doses of the first PTH compound of formula (I), active vitamin D and calcium supplement may be adjusted as described above.

Pharmaceutical compositions and presentations for the first PTH compound of formula (I) as used in steps (i-a) to (i-g) are as described elsewhere herein.

In certain embodiments the first PTH compound is AZP-3601, which may be administered at a dose ranging from 20 μg to 60 μg per day.

In step (b) the dose of the first PTH compound at which the patient's serum calcium level remains within normal range in the absence of active vitamin D and calcium supplement is used as the basis for determining the initial dose of the second PTH compound. This may be done in various ways and in the following some examples are described.

In the simplest case the active PTH of the first PTH compound is the same as the active PTH of the second PTH compound. In such case the dose of the first PTH compound at which the patient's serum calcium level remains within normal range in the absence of active vitamin D and calcium supplement, D₁, may be multiplied with the length of the second interval between administrations, T₂, divided by the lengths of the first interval between administrations, T₁, to obtain a dose D₂ of the second PTH compound:

$${D}_{2} = {D}_{1} \times \frac{{T}_{2}}{{T}_{1}}$$

D₁ may be given in mol or in PTH equivalents and D₂ will accordingly be in mol or in PTH equivalents.

The initial dose of the second PTH compound is D₂±25%. For practical reasons the initial dose is provided as a range, because the initial dose of the second PTH compound needs to fit the presentations of the second PTH compound available to the patient. For example, if D₂ is 152 μg and the available presentations of the second PTH compound only allow for administration in multiples of 10 μg, the initial dose of the second PTH compound may be either 150 μg or 160 μg. In certain embodiments D₂ is rounded to the closest administrable dose to give the initial dose of the second PTH compound, which may be higher or lower than D₂. In certain embodiments D₂ is rounded to the closest administrable dose lower than D₂ to give the initial dose of the second PTH compound. In certain embodiments D₂ is rounded to the closest administrable dose higher than D₂ to give the initial dose of the second PTH compound. Accordingly, in certain embodiments the initial dose of the second PTH compound administered in step (c) is D₂±25%. In certain embodiments the initial dose of the second PTH compound administered in step (c) is D₂±20. In certain embodiments the initial dose of the second PTH compound administered in step (c) falls into the range of D₂±15%. In certain embodiments the initial dose of the second PTH compound administered in step (c) falls is D₂±10%. In certain embodiments the initial dose of the second PTH compound administered in step (c) is D₂±5%. In certain embodiments the initial dose of the second PTH compound administered in step (c) is D₂.

It is understood that “D₂±10%” means that the initial dose of the second PTH compound administered in step (c) lies in the range from “D₂−10%” to “D₂+10%”. If for example D2 is calculated as 100 μg, the initial dose of the second PTH compound may in such case ranges from 90 μg to 110 μg.

If the active PTH of the first PTH compound is different from the active PTH of the second PTH compound, a conversion factor CF will first be determined to compensate for different potencies of the active PTH of the first and of the second PTH compound.

One way to obtain CF is by determining in a population of healthy volunteers the dose of the first PTH compound Dₛᵤₚₚ₁ that fully suppresses secretion of endogenous PTH 1-84 following administration of the first PTH compound on multiple occasions at the first average interval Tₛᵤₚₚ₁ for a first period of time and in addition determining in a population of healthy volunteers the dose of the second PTH compound Dₛᵤₚₚ₂ that fully suppresses secretion of endogenous PTH 1-84 following administration on multiple occasions of the second PTH compound at the second interval Tₛᵤₚₚ₂ for a second period of time. CF may be calculated by dividing Dₛᵤₚₚ₂ by Dₛᵤₚₚ₁ and multiplying with Tₛᵤₚₚ₁ divided by Tₛᵤₚₚ₂:

$$CF = \frac{{D}_{supp 2}}{{D}_{supp 1}} \times \frac{{T}_{supp 1}}{{T}_{supp 2}}$$

Dₛᵤₚₚ₁ and Dₛᵤₚₚ₂ may be given in mol or in PTH equivalents.

Dₛᵤₚₚ₁ and Dₛᵤₚₚ₂ are determined at steady state.

Determining Dₛᵤₚₚ₁ and Dₛᵤₚₚ₂ is part of the normal clinical trial program required for approval of a first or second PTH compound and thus does not require additional human trials.

If CF is available, D₂ may be calculated by multiplying D₁ with CF and T₁ divided by T₂:

$${D}_{2} = {D}_{1} \times C F \times \frac{{T}_{2}}{{T}_{1}}$$

In certain embodiments Ti corresponds to Tₛᵤₚₚ₁ and T₂ corresponds to Tₛᵤₚₚ₂.

If the doses of the first and the second PTH compound at which endogenous secretion of PTH 1-84 is suppressed are not available, D₂ may alternatively be determined by prediction human PK based on allometric scaling. This method works if the active PTH of the first PTH compound is the same as the active PTH for the second PTH compound. Accordingly, in certain embodiments D₂ may be calculated in step (b) by
(i) determining clearance and half-life of the second PTH compound in a non-human primate, such as for example in cynomolgus monkey, and using these values to predict the corresponding clearance and half-life of the second PTH compound in human using equation 1 and 2, respectively:

$$CL 2 human = CL 2 animal * {( \frac{Human body weight}{Animal body weight} )}^{SF CL} \qquad 1$$

$${t}_{1 / 2} 2 human = {t}_{1 / 2} 2 animal * {( \frac{Human body weight}{Animal body weight} )}^{SF T 1 / 2} \qquad 2$$

wherein
CL 2 human=clearance of the second PTH compound in human,
CL 2 animal=clearance of the second PTH compound in a non-human primate, such as for example in cynomolgus monkeys,
t_{1/2} 2 human=half-life of the second PTH compound in human, and
t_{1/2} 2 animal=half-life of the second PTH compound in a non-human primate, such as for example in cynomolgus monkeys,
SF CL=Scaling factor non-human primate for clearance, and
SF T½=Scaling factor non-human primate for half-life;
(ii) determining the dose of the first PTH compound at which the patient's serum calcium level remains within normal range in the absence of active vitamin D and calcium supplement (“Dose 1”) and measuring the respective concentration of the first PTH compound in blood (“C_{ave,ss}”); and
(iii) calculating Dose 2 of the second PTH compound using equation 3

$$Dose 2 = {C}_{ave , ss} * CL 2 human * S econd interval \qquad ( 3 )$$

wherein
C_{ave,ss}=concentration of the first PTH compound in blood,
CL 2 human=clearance of the second PTH compound in human,

$$Second interval = second interval between administrators .$$

As with the calculation described above, the initial dose of the second PTH compound is D₂ 25%. In certain embodiments the initial dose of the second PTH compound administered in step (c) is D₂+20%. In certain embodiments the initial dose of the second PTH compound administered in step (c) falls into the range of D₂+15%. In certain embodiments the initial dose of the second PTH compound administered in step (c) falls is D₂+10%. In certain embodiments the initial dose of the second PTH compound administered in step (c) is D₂+5%. In certain embodiments the initial dose of the second PTH compound administered in step (c) is D₂.

The scaling factors SF CL and SF T½ in equation 1 are available in the art. For example, SF CL is 0.85 and SF T½ is 0.15 (Antibodies 2022, 11(2), 42; https://www.mdpi.com/2073-4468/11/2/42).

Using the method based on the dose of the first and second compound that fully suppresses secretion of endogenous PTH in healthy volunteers provides more accurate calculation, because input values originate from human and not non-human primate.

In certain embodiments the time between the last dose of the first PTH compound and the first dose of the second PTH compound corresponds to the average interval between two administrations of the first PTH compound, i.e., there is no break between these two administrations. In certain embodiments the time between the last dose of the first PTH compound and the first dose of the second PTH compound corresponds to the interval between two administrations of the first PTH compound. In certain embodiments there is a break between the last dose of the first PTH compound and the first dose of second PTH compound, meaning the interval between the last dose of the first PTH compound and the first dose of the second PTH compound is larger than the first average interval between two administrations or larger than the first interval between two administrations of the first PTH compound. Such break may range from 6 hours to one week. In certain embodiments such break may range from 12 hours to 6 six days. In certain embodiments such break may range from one day to 5 days. In certain embodiments such break last for 12 hours. In certain embodiments such break lasts for one day. In certain embodiments such break lasts for two days. In certain embodiments such break lasts for three days. In certain embodiments such break lasts for four days. In certain embodiments such break lasts for five days. In certain embodiments such break lasts for six days. In certain embodiments such break lasts for seven days. Whenever there is a break between the last administration of the first PTH compound and the first administration of the second PTH compound the total time between these two administrations corresponds to the length of the average interval between two administrations for the first PTH compound or of the interval between two administrations for the first PTH compound extended by the time of the break.

In certain embodiments the second PTH compound may be administered to a patient by various modes of administration, such as via topical, enteral or parenteral administration and by methods of external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection and infusion, direct delivery to the brain via implanted device allowing delivery of the invention or the like to brain tissue or brain fluids (e.g., Ommaya Reservoir), direct intracerebroventricular injection or infusion, injection or infusion into brain or brain associated regions, injection into the subchoroidal space, retro-orbital injection and ocular instillation. In certain embodiments the second PTH compound is administered by subcutaneous, intramuscular or oral administration. In certain embodiments the second PTH compound is administered by subcutaneous administration, such as by subcutaneous injection.

In certain embodiments the second PTH compound is administered using a pen injector. In certain embodiments the second PTH compound is administered using a needle and syringe. In certain embodiments the second PTH compound is administered via subcutaneous injection using a pen injector. In certain embodiments the second PTH compound is administered via subcutaneous injection using a needle and syringe.

In certain embodiments the mode of administration for the first PTH compound and the second PTH compound are identical. In certain embodiments both the first PTH compound and the second PTH compound are administered via subcutaneous administration, such as subcutaneous injection. In certain embodiments both the first PTH compound and the second PTH compound are administered via subcutaneous injection with a pen injector. In certain embodiments both the first PTH compound and the second PTH compound are administered via subcutaneous injection with a needle and syringe.

In certain embodiments the second PTH compound is the only PTH-comprising drug administered to the patient during step (c).

In certain embodiments the first PTH compound is the only PTH-comprising drug administered to the patient during step (a) and the second PTH compound is the only PTH-comprising drug administered to the patient during step (c).

In certain embodiments the second average interval is at least 2-fold longer than the first average interval. In certain embodiments the second average interval is at least 3-fold longer than the first average interval. In certain embodiments the second average interval is at least 4-fold longer than the first average interval. In certain embodiments the second average interval is at least 5-fold longer than the first average interval.

In certain embodiments the second interval is at least 2-fold longer than the first interval. In certain embodiments the second interval is at least 3-fold longer than the first interval. In certain embodiments the second interval is at least 4-fold longer than the first interval. In certain embodiments the second interval is at least 5-fold longer than the first interval.

In certain embodiments the second average interval ranges from 2 days to one month. In certain embodiments the second average interval ranges from 2 days to one week. In certain embodiments the second average interval is approx. 2 days. In certain embodiments the second average interval is 2 days. In certain embodiments the second average interval is approx. 3 days. In certain embodiments the second average interval is 3 days. In certain embodiments the second average interval is approx. 4 days. In certain embodiments the second average interval is 4 days. In certain embodiments the second average interval is approx. 5 days. In certain embodiments the second average interval 5 days. In certain embodiments the second average interval is approx. 6 days. In certain embodiments the second average interval is 6 days. In certain embodiments the first average interval is approx. one week. In certain embodiments the second average interval is one week. In certain embodiments the second average interval in is approx. two weeks. In certain embodiments the second average interval is two weeks. In certain embodiments the second average interval is approx. three weeks. In certain embodiments the second average interval is three weeks. In certain embodiments the second average interval is approx. one month. In certain embodiments the second average interval is one month.

In certain embodiments the intervals between administrations in step (c) are of varying length.

In certain embodiments all intervals between administrations in step (c) have the same length. If all intervals between administrations have the same length, the length of such interval is referred to as “second interval between administration” or short “second interval”.

In certain embodiments the second interval ranges from days to one month. In certain embodiments the second interval ranges from days to one week. In certain embodiments the second interval is approx. 2 days. In certain embodiments the second interval is 2 days. In certain embodiments the second interval is approx. 3 days. In certain embodiments the second interval is 3 days. In certain embodiments the second interval is approx. 4 days. In certain embodiments the second interval is 4 days. In certain embodiments the second interval is approx. 5 days. In certain embodiments the second interval is 5 days. In certain embodiments the second interval is approx. 6 days. In certain embodiments the second is 6 days. In certain embodiments the first interval is approx. one week. In certain embodiments the second interval is one week. In certain embodiments the second interval is approx. two weeks. In certain embodiments the second interval is two weeks. In certain embodiments the second interval is approx. three weeks. In certain embodiments the second interval is three weeks. In certain embodiments the second interval is approx. one month. In certain embodiments the second interval is one month.

In certain embodiments the first average interval is approx. one day and the second average interval is approx. one week. In certain embodiments the first average interval is approx. 8 hours and the second average interval is approx. one week. In certain embodiments the first average interval is approx. 12 hours and the second average interval is approx. one week. In certain embodiments the first average interval is approx. one week and the second average interval is approx. one month. In certain embodiments the first average interval is one day and the second average interval is one week. In certain embodiments the first average interval is 8 hours and the second average interval is one week. In certain embodiments the first average interval is 12 hours and the second average interval is one week. In certain embodiments the first average interval is one week and the second average interval is one month.

In certain embodiments the first interval is approx. one day and the second interval is approx. one week. In certain embodiments the first interval is approx. 8 hours and the second interval is approx. one week. In certain embodiments the first interval is approx. 12 hours and the second interval is approx. one week. In certain embodiments the first interval is approx. one week and the second interval is approx. one month. In certain embodiments the first interval is one day and the second interval is one week. In certain embodiments the first interval is 8 hours and the second interval is one week. In certain embodiments the first interval is 12 hours and the second interval is one week. In certain embodiments the first interval is one week and the second interval is one month.

The second PTH compound is administered to the patient on multiple occasions in step (c) and the period of time from the first to the last occasion of administration of the second PTH compound in step (c) is referred to as “second treatment period”.

In certain embodiments the second treatment period lasts for as long as the patient benefits from administration of the second PTH compound. In certain embodiments the second treatment period lasts until the disease the can be treated with PTH is cured. In certain embodiments the second treatment period lasts until the end of the patient's life. In certain embodiments the second treatment period lasts for at least six months. In certain embodiments the second treatment period lasts for at least one year. In certain embodiments the second treatment period lasts for at least two years. In certain embodiments the second treatment period lasts for at least five years. In certain embodiments the second treatment period lasts for at least seven years. In certain embodiments the second treatment period lasts for at least ten years. In certain embodiments the second treatment period lasts for at least 15 years. In certain embodiments the second treatment period lasts for at least 20 years. In certain embodiments the second treatment period lasts for at least 25 years. In all cases treatment ends at the latest upon death of the patient.

In certain embodiments the dose of the second PTH compound is increased between at least two consecutive administrations in response to hypocalcemia. Upon increasing the dose, the patient may remain on said increased dose for one or more consecutive administrations or may remain on the increased dose for all administrations in the second treatment period. In certain embodiments the patient may after one or a few, such as two, three, four or five increased doses return to the prior, i.e., lower dose. In certain embodiments the dose of the second PTH compound is further increased between at least two consecutive administrations in response to persistent hypocalcemia. If necessary, further dose increases may be made.

In certain embodiments the dose of the second PTH compound is decreased between at least two consecutive administrations in response to hypercalcemia. Upon decreasing the dose, the patient may remain on said decreased dose for one or more consecutive administrations or may remain on the decreased dose for all administrations in the second treatment period. In certain embodiments the patient may after one or a few, such as two, three, four or five decreased doses return to the prior, i.e., higher dose. In certain embodiments the dose of the second PTH compound is further decreased between at least two consecutive administrations in response to persistent hypercalcemia. If necessary, further dose decreases may be made.

It is understood that step (c) may comprise any combination of increases and decreases of the dose of the second PTH compound.

It is understood that once the patient in step (c) has become a stable patient on the second PTH compound, treatment with the second PTH compound continues for as long as the patient may benefit from such treatment. The dose of the second PTH compound on which the patient is stable may also be referred to as “maintenance dose”. Accordingly, once the patient in step (c) has become a stable patient on the second PTH compound, treatment with the second PTH compound continues with the maintenance dose. Optionally, the maintenance dose is increased in response to hypocalcemia and/or the maintenance dose is decreased in response to hypercalcemia.

In certain embodiments the second PTH compound is a compound of formula (II-a)

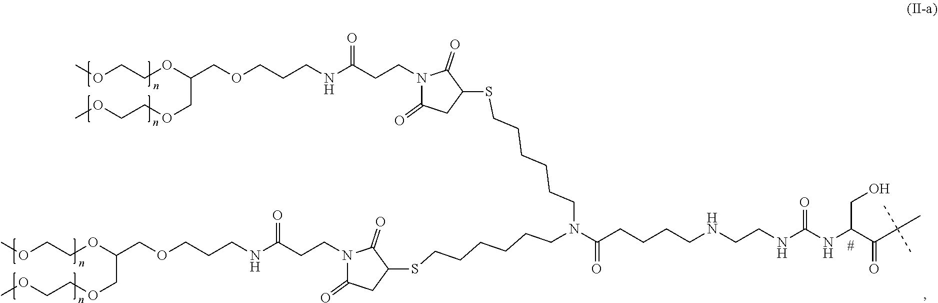

wherein
the dashed line indicates attachment to the nitrogen of the N-terminal amine group of a PTH moiety having the sequence of SEQ ID NO:51; and
each n is independently an integer ranging from approx. 200 to 250.

The chiral center marked with “#” in formula (II-a) may be in L- or D-configuration or may be a mixture of both L- and D-configuration. In certain embodiments said chiral center is in L-configuration.

In certain embodiments the initial dose of compound (II-a) in step (c) ranges from 100 μg to 250 μg/week.

In certain embodiments the dose at which the patient's serum calcium level remains within normal range in the absence of active vitamin D and calcium supplement in step (a) is 18 μg and the initial dose of compound (II-a) in step (c) ranges from 101.25 μg to 168.75 μg/week.

In certain embodiments the dose at which the patient's serum calcium level remains within normal range in the absence of active vitamin D and calcium supplement in step (a) is 18 μg and the initial dose of compound (II-a) in step (c) ranges from 108 μg to 162 μg/week. In certain embodiments the dose at which the patient's serum calcium level remains within normal range in the absence of active vitamin D and calcium supplement in step (a) is 18 μg and the initial dose of compound (II-a) in step (c) ranges from 114.75 μg to 155.25 μg/week. In certain embodiments the dose at which the patient's serum calcium level remains within normal range in the absence of active vitamin D and calcium supplement in step (a) is 18 μg and the initial dose of compound (II-a) in step (c) ranges from 121.5 μg to 148.5 μg/week. In certain embodiments the dose at which the patient's serum calcium level remains within normal range in the absence of active vitamin D and calcium supplement in step (a) is 18 μg and the initial dose of compound (II-a) in step (c) ranges from 128.25 μg to 141.75 μg/week. In certain embodiments the dose at which the patient's serum calcium level remains within normal range in the absence of active vitamin D and calcium supplement in step (a) is 18 μg and the initial dose of compound (II-a) in step (c) is 135 μg/week.

In certain embodiments the dose at which the patient's serum calcium level remains within normal range in the absence of active vitamin D and calcium supplement in step (a) is 21 μg and the initial dose of compound (II-a) in step (c) ranges from 118.5 μg to 197.5 μg/week. In certain embodiments the dose at which the patient's serum calcium level remains within normal range in the absence of active vitamin D and calcium supplement in step (a) is 21 μg and the initial dose of compound (II-a) in step (c) ranges from 126.4 μg to 189.6 μg/week. In certain embodiments the dose at which the patient's serum calcium level remains within normal range in the absence of active vitamin D and calcium supplement in step (a) is 21 μg and the initial dose of compound (II-a) in step (c) ranges from 134.3 μg to 181.7 μg/week. In certain embodiments the dose at which the patient's serum calcium level remains within normal range in the absence of active vitamin D and calcium supplement in step (a) is 21 μg and the initial dose of compound (II-a) in step (c) ranges from 142.2 μg to 173.8 μg/week. In certain embodiments the dose at which the patient's serum calcium level remains within normal range in the absence of active vitamin D and calcium supplement in step (a) is 21 μg and the initial dose of compound (II-a) in step (c) ranges from 150.1 μg to 165.9 μg/week. In certain embodiments the dose at which the patient's serum calcium level remains within normal range in the absence of active vitamin D and calcium supplement in step (a) is 21 μg and the initial dose of compound (II-a) in step (c) is 158 μg/week.

In certain embodiments the dose at which the patient's serum calcium level remains within normal range in the absence of active vitamin D and calcium supplement in step (a) is 24 μg and the initial dose of compound (II-a) in step (c) ranges from 135 μg to 225 μg/week. In certain embodiments the dose at which the patient's serum calcium level remains within normal range in the absence of active vitamin D and calcium supplement in step (a) is 24 μg and the initial dose of compound (II-a) in step (c) ranges from 144 μg to 216 μg/week. In certain embodiments the dose at which the patient's serum calcium level remains within normal range in the absence of active vitamin D and calcium supplement in step (a) is 24 μg and the initial dose of compound (II-a) in step (c) ranges from 153 μg to 207 μg/week. In certain embodiments the dose at which the patient's serum calcium level remains within normal range in the absence of active vitamin D and calcium supplement in step (a) is 24 μg and the initial dose of compound (II-a) in step (c) ranges from 162 μg to 198 μg/week. In certain embodiments the dose at which the patient's serum calcium level remains within normal range in the absence of active vitamin D and calcium supplement in step (a) is 24 μg and the initial dose of compound (II-a) in step (c) ranges from 172 μg to 189 μg/week. In certain embodiments the dose at which the patient's serum calcium level remains within normal range in the absence of active vitamin D and calcium supplement in step (a) is 24 μg and the initial dose of compound (II-a) in step (c) is 180 μg/week.

In certain embodiments the second PTH compound is a compound of formula (II-a′)

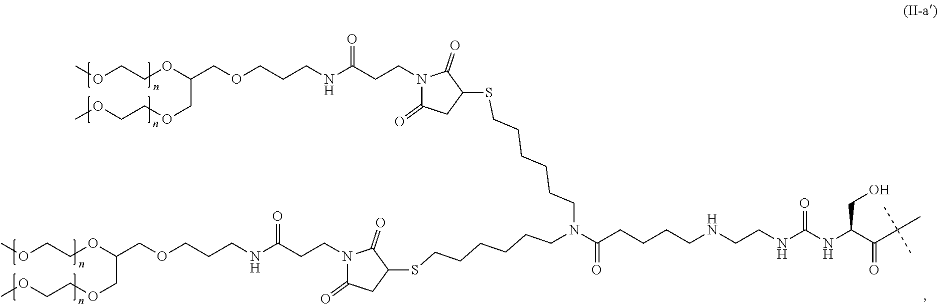

wherein
   the dashed line indicates attachment to the nitrogen of the N-terminal amine group of a PTH moiety having the sequence of SEQ ID NO:51; and
each n is independently an integer ranging from approx. 200 to 250.

In certain embodiments the initial dose of compound (II-a′) in step (c) ranges from 100 μg to 250 μg/week.

In certain embodiments the dose at which the patient's serum calcium level remains within normal range in the absence of active vitamin D and calcium supplement in step (a) is 18 μg and the initial dose of compound (II-a′) in step (c) ranges from 101.25 μg to 168.75 μg/week. In certain embodiments the dose at which the patient's serum calcium level remains within normal range in the absence of active vitamin D and calcium supplement in step (a) is 18 μg and the initial dose of compound (II-a′) in step (c) ranges from 108 μg to 162 μg/week. In certain embodiments the dose at which the patient's serum calcium level remains within normal range in the absence of active vitamin D and calcium supplement in step (a) is 18 μg and the initial dose of compound (II-a′) in step (c) ranges from 114.75 μg to 155.25 μg/week. In certain embodiments the dose at which the patient's serum calcium level remains within normal range in the absence of active vitamin D and calcium supplement in step (a) is 18 μg and the initial dose of compound (II-a′) in step (c) ranges from 121.5 μg to 148.5 μg/week. In certain embodiments the dose at which the patient's serum calcium level remains within normal range in the absence of active vitamin D and calcium supplement in step (a) is 18 μg and the initial dose of compound (II-a′) in step (c) ranges from 128.25 μg to 141.75 μg/week. In certain embodiments the dose at which the patient's serum calcium level remains within normal range in the absence of active vitamin D and calcium supplement in step (a) is 18 μg and the initial dose of compound (II-a′) in step (c) is 135 μg/week.

In certain embodiments the dose at which the patient's serum calcium level remains within normal range in the absence of active vitamin D and calcium supplement in step (a) is 21 μg and the initial dose of compound (II-a′) in step (c) ranges from 118.5 μg to 197.5 μg/week. In certain embodiments the dose at which the patient's serum calcium level remains within normal range in the absence of active vitamin D and calcium supplement in step (a) is 21 μg and the initial dose of compound (II-a′) in step (c) ranges from 126.4 μg to 189.6 μg/week. In certain embodiments the dose at which the patient's serum calcium level remains within normal range in the absence of active vitamin D and calcium supplement in step (a) is 21 μg and the initial dose of compound (II-a′) in step (c) ranges from 134.3 μg to 181.7 μg/week. In certain embodiments the dose at which the patient's serum calcium level remains within normal range in the absence of active vitamin D and calcium supplement in step (a) is 21 μg and the initial dose of compound (II-a′) in step (c) ranges from 142.2 μg to 173.8 μg/week. In certain embodiments the dose at which the patient's serum calcium level remains within normal range in the absence of active vitamin D and calcium supplement in step (a) is 21 μg and the initial dose of compound (II-a′) in step (c) ranges from 150.1 μg to 165.9 μg/week. In certain embodiments the dose at which the patient's serum calcium level remains within normal range in the absence of active vitamin D and calcium supplement in step (a) is 21 μg and the initial dose of compound (II-a′) in step (c) is 158 μg/week.

In certain embodiments the dose at which the patient's serum calcium level remains within normal range in the absence of active vitamin D and calcium supplement in step (a) is 24 μg and the initial dose of compound (II-a′) in step (c) ranges from 135 μg to 225 μg/week. In certain embodiments the dose at which the patient's serum calcium level remains within normal range in the absence of active vitamin D and calcium supplement in step (a) is 24 μg and the initial dose of compound (II-a′) in step (c) ranges from 144 μg to 216 μg/week. In certain embodiments the dose at which the patient's serum calcium level remains within normal range in the absence of active vitamin D and calcium supplement in step (a) is 24 μg and the initial dose of compound (II-a′) in step (c) ranges from 153 μg to 207 μg/week. In certain embodiments the dose at which the patient's serum calcium level remains within normal range in the absence of active vitamin D and calcium supplement in step (a) is 24 μg and the initial dose of compound (II-a′) in step (c) ranges from 162 μg to 198 μg/week. In certain embodiments the dose at which the patient's serum calcium level remains within normal range in the absence of active vitamin D and calcium supplement in step (a) is 24 μg and the initial dose of compound (II-a′) in step (c) ranges from 172 μg to 189 μg/week. In certain embodiments the dose at which the patient's serum calcium level remains within normal range in the absence of active vitamin D and calcium supplement in step (a) is 24 μg and the initial dose of compound (II-a′) in step (c) is 180 μg/week.

In certain embodiments each n of formula (II-a) and (II-a′) is independently an integer ranging from 200 to 250. In certain embodiments each n of formula (II-a) and (II-a′) is independently an integer ranging from approx. 210 to 240. In certain embodiments each n of formula (II-a) and (II-a′) is independently an integer ranging from 210 to 240.

It is understood that the nitrogen of the N-terminal amine group of the PTH moiety and the carbonyl (—(C═O)—) group in formula (II-a) and (II-a′) to the left of the dashed line form an amide bond.

The PTH compound of formula (II-a) and (II-a′) releases PTH of SEQ ID NO:51, PTH 1-34, meaning that the active PTH of the PTH compound of formula (II-a) and (II-a′) is PTH 1-34.

In certain embodiments the second PTH compound is a compound of formula (II-a) and the second average interval is approx. one week. In certain embodiments the second PTH compound is a compound of formula (II-a) and the second average interval is one week. In certain embodiments the second PTH compound is a compound of formula (II-a), the second average interval is one week and administration is via subcutaneous injection. In certain embodiments the second PTH compound is a compound of formula (II-a), the second average interval is one week and administration is via subcutaneous injection with a pen injector. In certain embodiments the second PTH compound is a compound of formula (II-a) with each n being independently an integer ranging from 200 to 250, the second average interval is one week and administration is via subcutaneous injection with a pen injector.

In certain embodiments the second PTH compound is a compound of formula (II-a′) and the second average interval is approx. one week. In certain embodiments the second PTH compound is a compound of formula (II-a′) and the second average interval is one week. In certain embodiments the second PTH compound is a compound of formula (II-a′), the second average interval is one week and administration is via subcutaneous injection. In certain embodiments the second PTH compound is a compound of formula (II-a′), the second average interval is one week and administration is via subcutaneous injection with a pen injector. In certain embodiments the second PTH compound is a compound of formula (II-a′) with each n being independently an integer ranging from 200 to 250, the second average interval is one week and administration is via subcutaneous injection with a pen injector.

In certain embodiments the second PTH compound is a compound of formula (II-a) and the second interval is approx. one week. In certain embodiments the second PTH compound is a compound of formula (II-a) and the second interval is one week. In certain embodiments the second PTH compound is a compound of formula (II-a), the second interval is one week and administration is via subcutaneous injection. In certain embodiments the second PTH compound is a compound of formula (II-a), the second interval is one week and administration is via subcutaneous injection with a pen injector. In certain embodiments the second PTH compound is a compound of formula (II-a) with each n being independently an integer ranging from 200 to 250, the second interval is one week and administration is via subcutaneous injection with a pen injector.

In certain embodiments the second PTH compound is a compound of formula (II-a′) and the second interval is approx. one week. In certain embodiments the second PTH compound is a compound of formula (II-a′) and the second interval is one week. In certain embodiments the second PTH compound is a compound of formula (II-a′), the second interval is one week and administration is via subcutaneous injection. In certain embodiments the second PTH compound is a compound of formula (II-a′), the second interval is one week and administration is via subcutaneous injection with a pen injector. In certain embodiments the second PTH compound is a compound of formula (II-a′) with each n being independently an integer ranging from 200 to 250, the second interval is one week and administration is via subcutaneous injection with a pen injector.

In certain embodiments the first PTH compound is of formula (I), the first average interval between administrations is approx. one day, the second PTH compound is of formula (II-a) and the second average interval between administrations is approx. one week. In certain embodiments the first PTH compound is of formula (I), the first average interval between administrations is one day, the second PTH compound is of formula (II-a) and the second average interval between administrations is one week. In certain embodiments the first PTH compound is of formula (I), the first interval between administrations is approx. one day, the second PTH compound is of formula (II-a) and the second interval between administrations is approx. one week. In certain embodiments the first PTH compound is of formula (I), the first interval between administrations is one day, the second PTH compound is of formula (II-a) and the second interval between administrations is one week. In certain embodiments the first PTH compound is of formula (I) with m and p independently being an integer ranging from approx. 450 to 500, the first interval between administrations is one day, the second PTH compound is of formula (II-a) with each n independently being an integer ranging from approx. 200 to 250 and the second interval between administrations is one week. In certain embodiments the first PTH compound is of formula (I) with m and p independently being an integer ranging from 450 to 500, the first interval between administrations is one day, the second PTH compound is of formula (II-a) with each n independently being an integer ranging from 200 to 250 and the second interval between administrations is one week. In certain embodiments the first PTH compound is of formula (I) with m and p independently being an integer ranging from approx. 450 to 500, the first interval between administrations is one day, the second PTH compound is of formula (II-a) with each n independently being an integer ranging from 210 to 240 and the second interval between administrations is one week. In certain embodiments the first PTH compound is of formula (I) with m and p independently being an integer ranging from 450 to 500, the first interval between administrations is one day, the second PTH compound is of formula (II-a) with each n independently being an integer ranging from 210 to 240 and the second interval between administrations is one week.

In certain embodiments the first PTH compound is of formula (I), the first average interval between administrations is approx. one day, the second PTH compound is of formula (II-a′) and the second average interval between administrations is approx. one week. In certain embodiments the first PTH compound is of formula (I), the first average interval between administrations is one day, the second PTH compound is of formula (II-a′) and the second average interval between administrations is one week. In certain embodiments the first PTH compound is of formula (I), the first interval between administrations is approx. one day, the second PTH compound is of formula (II-a′) and the second interval between administrations is approx. one week. In certain embodiments the first PTH compound is of formula (I), the first interval between administrations is one day, the second PTH compound is of formula (II-a′) and the second interval between administrations is one week. In certain embodiments the first PTH compound is of formula (I) with m and p independently being an integer ranging from approx. 450 to 500, the first interval between administrations is one day, the second PTH compound is of formula (II-a′) with each n independently being an integer ranging from approx. 200 to 250 and the second interval between administrations is one week. In certain embodiments the first PTH compound is of formula (I) with m and p independently being an integer ranging from 450 to 500, the first interval between administrations is one day, the second PTH compound is of formula (II-a′) with each n independently being an integer ranging from 200 to 250 and the second interval between administrations is one week. In certain embodiments the first PTH compound is of formula (I) with m and p independently being an integer ranging from approx. 450 to 500, the first interval between administrations is one day, the second PTH compound is of formula (II-a′) with each n independently being an integer ranging from 210 to 240 and the second interval between administrations is one week. In certain embodiments the first PTH compound is of formula (I) with m and p independently being an integer ranging from 450 to 500, the first interval between administrations is one day, the second PTH compound is of formula (II-a′) with each n independently being an integer ranging from 210 to 240 and the second interval between administrations is one week.

In certain embodiments the second PTH compound is a compound of formula (II-b)

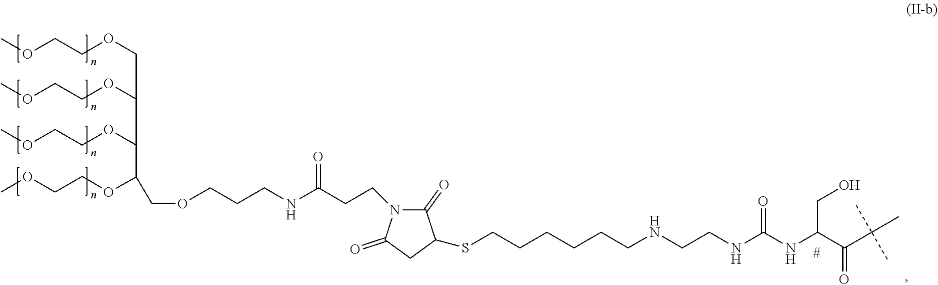

wherein
the dashed line indicates attachment to the nitrogen of the N-terminal amine group of a PTH moiety having the sequence of SEQ ID NO:51; and
each n is independently an integer ranging from approx. 200 to 250.

The chiral center marked with “#” in formula (II-b) may be in L- or D-configuration or may be a mixture of both L- and D-configuration. In certain embodiments said chiral center is in L-configuration.

In certain embodiments the initial dose of compound (II-b) in step (c) ranges from 85 μg to 200 μg/week.

In certain embodiments the dose at which the patient's serum calcium level remains within normal range in the absence of active vitamin D and calcium supplement in step (a) is 18 μg and the initial dose of compound (II-b) in step (c) ranges from 87 μg to 145 μg/week. In certain embodiments the dose at which the patient's serum calcium level remains within normal range in the absence of active vitamin D and calcium supplement in step (a) is 18 μg and the initial dose of compound (II-b) in step (c) ranges from 92.8 μg to 139.2 μg/week. In certain embodiments the dose at which the patient's serum calcium level remains within normal range in the absence of active vitamin D and calcium supplement in step (a) is 18 μg and the initial dose of compound (II-b) in step (c) ranges from 98.6 μg to 133.4 μg/week. In certain embodiments the dose at which the patient's serum calcium level remains within normal range in the absence of active vitamin D and calcium supplement in step (a) is 18 μg and the initial dose of compound (II-b) in step (c) ranges from 104.4 μg to 127.6 μg/week. In certain embodiments the dose at which the patient's serum calcium level remains within normal range in the absence of active vitamin D and calcium supplement in step (a) is 18 μg and the initial dose of compound (II-b) in step (c) ranges from 110.2 μg to 121.8 μg/week. In certain embodiments the dose at which the patient's serum calcium level remains within normal range in the absence of active vitamin D and calcium supplement in step (a) is 18 μg and the initial dose of compound (II-b) in step (c) is 116 μg/week.

In certain embodiments the dose at which the patient's serum calcium level remains within normal range in the absence of active vitamin D and calcium supplement in step (a) is 21 μg and the initial dose of compound (II-b) in step (c) ranges from 101.25 μg to 168.75 μg/week. In certain embodiments the dose at which the patient's serum calcium level remains within normal range in the absence of active vitamin D and calcium supplement in step (a) is 21 μg and the initial dose of compound (II-b) in step (c) ranges from 108 μg to 162 μg/week. In certain embodiments the dose at which the patient's serum calcium level remains within normal range in the absence of active vitamin D and calcium supplement in step (a) is 21 μg and the initial dose of compound (II-b) in step (c) ranges from 114.75 μg to 155.25 μg/week. In certain embodiments the dose at which the patient's serum calcium level remains within normal range in the absence of active vitamin D and calcium supplement in step (a) is 21 μg and the initial dose of compound (II-b) in step (c) ranges from 121.5 μg to 148 μg/week. In certain embodiments the dose at which the patient's serum calcium level remains within normal range in the absence of active vitamin D and calcium supplement in step (a) is 21 μg and the initial dose of compound (II-b) in step (c) ranges from 128.25 μg to 141.75 μg/week. In certain embodiments the dose at which the patient's serum calcium level remains within normal range in the absence of active vitamin D and calcium supplement in step (a) is 21 μg and the initial dose of compound (II-b) in step (c) is 135 μg/week.

In certain embodiments the dose at which the patient's serum calcium level remains within normal range in the absence of active vitamin D and calcium supplement in step (a) is 24 μg and the initial dose of compound (II-b) in step (c) ranges from 115.5 μg to 192.5 μg/week. In certain embodiments the dose at which the patient's serum calcium level remains within normal range in the absence of active vitamin D and calcium supplement in step (a) is 24 μg and the initial dose of compound (II-b) in step (c) ranges from 123.5 μg to 184.5 μg/week. In certain embodiments the dose at which the patient's serum calcium level remains within normal range in the absence of active vitamin D and calcium supplement in step (a) is 24 μg and the initial dose of compound (II-b) in step (c) ranges from 130.9 μg to 177.1 μg/week. In certain embodiments the dose at which the patient's serum calcium level remains within normal range in the absence of active vitamin D and calcium supplement in step (a) is 24 μg and the initial dose of compound (II-b) in step (c) ranges from 138.6 μg to 169.4 μg/week. In certain embodiments the dose at which the patient's serum calcium level remains within normal range in the absence of active vitamin D and calcium supplement in step (a) is 24 μg and the initial dose of compound (II-b) in step (c) ranges from 146.3 μg to 161.7 μg/week. In certain embodiments the dose at which the patient's serum calcium level remains within normal range in the absence of active vitamin D and calcium supplement in step (a) is 24 μg and the initial dose of compound (II-b) in step (c) is 154 μg/week.

In certain embodiments the second PTH compound is a compound of formula (II-b′)

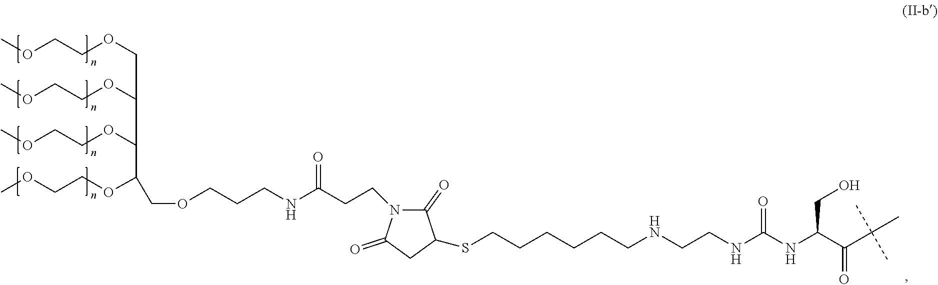

wherein
the dashed line indicates attachment to the nitrogen of the N-terminal amine group of a PTH moiety having the sequence of SEQ ID NO:51; and
each n is independently an integer ranging from approx. 200 to 250.

In certain embodiments each n of formula (II-b) and (II-b′) is independently an integer ranging from 200 to 250. In certain embodiments each n of formula (II-b) and (II-b′) is independently an integer ranging from approx. 210 to 240. In certain embodiments each n of formula (II-b) and (II-b′) is independently an integer ranging from 210 to 240.

It is understood that the nitrogen of the N-terminal amine group of the PTH moiety and the carbonyl (—(C═O)—) group in formula (II-b) and (II-b′) to the left of the dashed line form an amide bond.

The PTH compound of formula (II-b) and (II-b′) releases PTH of SEQ ID NO:51, PTH 1-34, meaning that the active PTH of the PTH compound of formula (II-b) and (II-b′) is PTH 1-34.

In certain embodiments the initial dose of compound (II-b′) in step (c) ranges from 85 μg to 200 μg/week.

In certain embodiments the dose at which the patient's serum calcium level remains within normal range in the absence of active vitamin D and calcium supplement in step (a) is 18 μg and the initial dose of compound (II-b′) in step (c) ranges from 87 μg to 145 μg/week. In certain embodiments the dose at which the patient's serum calcium level remains within normal range in the absence of active vitamin D and calcium supplement in step (a) is 18 μg and the initial dose of compound (II-b′) in step (c) ranges from 92.8 μg to 139.2 μg/week. In certain embodiments the dose at which the patient's serum calcium level remains within normal range in the absence of active vitamin D and calcium supplement in step (a) is 18 μg and the initial dose of compound (II-b′) in step (c) ranges from 98.6 μg to 133.4 μg/week. In certain embodiments the dose at which the patient's serum calcium level remains within normal range in the absence of active vitamin D and calcium supplement in step (a) is 18 μg and the initial dose of compound (II-b′) in step (c) ranges from 104.4 μg to 127.6 μg/week. In certain embodiments the dose at which the patient's serum calcium level remains within normal range in the absence of active vitamin D and calcium supplement in step (a) is 18 μg and the initial dose of compound (II-b′) in step (c) ranges from 110.2 μg to 121.8 μg/week. In certain embodiments the dose at which the patient's serum calcium level remains within normal range in the absence of active vitamin D and calcium supplement in step (a) is 18 μg and the initial dose of compound (II-b′) in step (c) is 116 μg/week.

In certain embodiments the dose at which the patient's serum calcium level remains within normal range in the absence of active vitamin D and calcium supplement in step (a) is 21 μg and the initial dose of compound (II-b′) in step (c) ranges from 101.25 μg to 168.75 μg/week. In certain embodiments the dose at which the patient's serum calcium level remains within normal range in the absence of active vitamin D and calcium supplement in step (a) is 21 μg and the initial dose of compound (II-b′) in step (c) ranges from 108 μg to 162 μg/week. In certain embodiments the dose at which the patient's serum calcium level remains within normal range in the absence of active vitamin D and calcium supplement in step (a) is 21 μg and the initial dose of compound (II-b′) in step (c) ranges from 114.75 μg to 155.25 μg/week. In certain embodiments the dose at which the patient's serum calcium level remains within normal range in the absence of active vitamin D and calcium supplement in step (a) is 21 μg and the initial dose of compound (II-b′) in step (c) ranges from 121.5 μg to 148 μg/week. In certain embodiments the dose at which the patient's serum calcium level remains within normal range in the absence of active vitamin D and calcium supplement in step (a) is 21 μg and the initial dose of compound (II-b′) in step (c) ranges from 128.25 μg to 141.75 μg/week. In certain embodiments the dose at which the patient's serum calcium level remains within normal range in the absence of active vitamin D and calcium supplement in step (a) is 21 μg and the initial dose of compound (II-b′) in step (c) is 135 μg/week.

In certain embodiments the dose at which the patient's serum calcium level remains within normal range in the absence of active vitamin D and calcium supplement in step (a) is 24 μg and the initial dose of compound (II-b′) in step (c) ranges from 115.5 μg to 192.5 μg/week. In certain embodiments the dose at which the patient's serum calcium level remains within normal range in the absence of active vitamin D and calcium supplement in step (a) is 24 μg and the initial dose of compound (II-b′) in step (c) ranges from 123.5 μg to 184.5 μg/week. In certain embodiments the dose at which the patient's serum calcium level remains within normal range in the absence of active vitamin D and calcium supplement in step (a) is 24 μg and the initial dose of compound (II-b′) in step (c) ranges from 130.9 μg to 177.1 μg/week. In certain embodiments the dose at which the patient's serum calcium level remains within normal range in the absence of active vitamin D and calcium supplement in step (a) is 24 μg and the initial dose of compound (II-b′) in step (c) ranges from 138.6 μg to 169.4 μg/week. In certain embodiments the dose at which the patient's serum calcium level remains within normal range in the absence of active vitamin D and calcium supplement in step (a) is 24 μg and the initial dose of compound (II-b′) in step (c) ranges from 146.3 μg to 161.7 μg/week. In certain embodiments the dose at which the patient's serum calcium level remains within normal range in the absence of active vitamin D and calcium supplement in step (a) is 24 μg and the initial dose of compound (II-b′) in step (c) is 154 μg/week.

In certain embodiments the second PTH compound is a compound of formula (II-b) and the second average interval is approx. one week. In certain embodiments the second PTH compound is a compound of formula (II-b) and the second average interval is one week. In certain embodiments the second PTH compound is a compound of formula (II-b), the second average interval is one week and administration is via subcutaneous injection. In certain embodiments the second PTH compound is a compound of formula (II-b), the second average interval is one week and administration is via subcutaneous injection with a pen injector. In certain embodiments the second PTH compound is a compound of formula (II-b) with each n being independently an integer ranging from 200 to 250, the second average interval is one week and administration is via subcutaneous injection with a pen injector.

In certain embodiments the second PTH compound is a compound of formula (II-b′) and the second average interval is approx. one week. In certain embodiments the second PTH compound is a compound of formula (II-b′) and the second average interval is one week. In certain embodiments the second PTH compound is a compound of formula (II-b′), the second average interval is one week and administration is via subcutaneous injection. In certain embodiments the second PTH compound is a compound of formula (II-b′), the second average interval is one week and administration is via subcutaneous injection with a pen injector. In certain embodiments the second PTH compound is a compound of formula (II-b′) with each n being independently an integer ranging from 200 to 250, the second average interval is one week and administration is via subcutaneous injection with a pen injector.

In certain embodiments the second PTH compound is a compound of formula (II-b) and the second interval is approx. one week. In certain embodiments the second PTH compound is a compound of formula (II-b) and the second interval is one week. In certain embodiments the second PTH compound is a compound of formula (II-b), the second interval is one week and administration is via subcutaneous injection. In certain embodiments the second PTH compound is a compound of formula (II-b), the second interval is one week and administration is via subcutaneous injection with a pen injector. In certain embodiments the second PTH compound is a compound of formula (II-b) with each n being independently an integer ranging from 200 to 250, the second interval is one week and administration is via subcutaneous injection with a pen injector.

In certain embodiments the second PTH compound is a compound of formula (II-b′) and the second interval is approx. one week. In certain embodiments the second PTH compound is a compound of formula (II-b′) and the second interval is one week. In certain embodiments the second PTH compound is a compound of formula (II-b′), the second interval is one week and administration is via subcutaneous injection. In certain embodiments the second PTH compound is a compound of formula (II-b′), the second interval is one week and administration is via subcutaneous injection with a pen injector. In certain embodiments the second PTH compound is a compound of formula (II-b′) with each n being independently an integer ranging from 200 to 250, the second interval is one week and administration is via subcutaneous injection with a pen injector.

In certain embodiments the first PTH compound is of formula (I), the first average interval between administrations is approx. one day, the second PTH compound is of formula (II-b) and the second average interval between administrations is approx. one week. In certain embodiments the first PTH compound is of formula (I), the first average interval between administrations is one day, the second PTH compound is of formula (II-b) and the second average interval between administrations is one week. In certain embodiments the first PTH compound is of formula (I), the first interval between administrations is approx. one day, the second PTH compound is of formula (II-b) and the second interval between administrations is approx. one week. In certain embodiments the first PTH compound is of formula (I), the first interval between administrations is one day, the second PTH compound is of formula (II-b) and the second interval between administrations is one week. In certain embodiments the first PTH compound is of formula (I) with m and p independently being an integer ranging from approx. 450 to 500, the first interval between administrations is one day, the second PTH compound is of formula (II-b) with each n independently being an integer ranging from approx. 200 to 250 and the second interval between administrations is one week. In certain embodiments the first PTH compound is of formula (I) with m and p independently being an integer ranging from 450 to 500, the first interval between administrations is one day, the second PTH compound is of formula (II-b) with each n independently being an integer ranging from 200 to 250 and the second interval between administrations is one week. In certain embodiments the first PTH compound is of formula (I) with m and p independently being an integer ranging from approx. 450 to 500, the first interval between administrations is one day, the second PTH compound is of formula (II-b) with each n independently being an integer ranging from 210 to 240 and the second interval between administrations is one week. In certain embodiments the first PTH compound is of formula (I) with m and p independently being an integer ranging from 450 to 500, the first interval between administrations is one day, the second PTH compound is of formula (II-b) with each n independently being an integer ranging from 210 to 240 and the second interval between administrations is one week.

In certain embodiments the first PTH compound is of formula (I), the first average interval between administrations is approx. one day, the second PTH compound is of formula (II-b′) and the second average interval between administrations is approx. one week. In certain embodiments the first PTH compound is of formula (I), the first average interval between administrations is one day, the second PTH compound is of formula (II-b′) and the second average interval between administrations is one week. In certain embodiments the first PTH compound is of formula (I), the first interval between administrations is approx. one day, the second PTH compound is of formula (II-b′) and the second interval between administrations is approx. one week. In certain embodiments the first PTH compound is of formula (I), the first interval between administrations is one day, the second PTH compound is of formula (II-b′) and the second interval between administrations is one week. In certain embodiments the first PTH compound is of formula (I) with m and p independently being an integer ranging from approx. 450 to 500, the first interval between administrations is one day, the second PTH compound is of formula (II-b′) with each n independently being an integer ranging from approx. 200 to 250 and the second interval between administrations is one week. In certain embodiments the first PTH compound is of formula (I) with m and p independently being an integer ranging from 450 to 500, the first interval between administrations is one day, the second PTH compound is of formula (II-b′) with each n independently being an integer ranging from 200 to 250 and the second interval between administrations is one week. In certain embodiments the first PTH compound is of formula (I) with m and p independently being an integer ranging from approx. 450 to 500, the first interval between administrations is one day, the second PTH compound is of formula (II-b′) with each n independently being an integer ranging from 210 to 240 and the second interval between administrations is one week. In certain embodiments the first PTH compound is of formula (I) with m and p independently being an integer ranging from 450 to 500, the first interval between administrations is one day, the second PTH compound is of formula (II-b′) with each n independently being an integer ranging from 210 to 240 and the second interval between administrations is one week.

In certain embodiments the second PTH compound is a compound of formula (II-i)

(II-i)
k(γE-(miniPEG)₂-γE-COC₁₆H₃₂CO₂H)(N-Me)GSVSEIQL
MHNLGKHLNSMERVEWLRKKLQDVHK(γE-(miniPEG)₂-γE-
COC₁₆H₃₂CO₂H)-OH,

wherein
k is d-Lys;
γE is the 1-isomer of gamma, glutamic acid;
miniPEG is COCH₂OCH₂CH₂OCH₂CH₂NH;
COC₁₆H₃₂CO₂H is C18 diacid;
(N-Me)G is sarcosine;
K is l-isomer of lysine; and
—OH designates the C-terminal amino acid has a terminal carboxylic acid.

In certain embodiments the second PTH compound is compound of formula (II-i′)

(II-i′)
k(γE-(miniPEG)₂-γE-COC₁₆H₃₂CO₂H)(N-Me)GSVSEIQL
MHNLGKHLNSMERVEWLRKKLQDVHK(γE-(miniPEG)₂-γE-
COC₁₆H₃₂CO₂H)-OH,

wherein
k is d-Lys;
γE is the 1-isomer of gamma, glutamic acid;
(miniPEG)₂ is COCH₂OCH₂CH₂OCH₂CH₂NH;
COC₁₆H₃₂CO₂H is C18 diacid;
(N-Me)G is sarcosine;
K is 1-isomer of lysine; and
—OH designates the C-terminal amino acid has a terminal carboxylic acid.

The compound of formula (II-i′) corresponds to SEQ ID NO:87 of WO2021/242756 and the sequence “SVSEIQLMHNLGKHLNSMERVEWLRKKLQDVHK” corresponds to PTH (1-32) (SEQ ID NO:53) herein+K33.

The compound of formula (II-i) release a compound of formula (II-ii)

(II-ii)
SVSEIQLMHNLGKHLNSMERVEWLRKKLQDVHK(γE-
(miniPEG)₂-γE-COC₁₆H₃₂CO₂H)-OH,

wherein
γE is the 1-isomer of gamma, glutamic acid;
miniPEG is COCH₂OCH₂CH₂OCH₂CH₂NH;
COC₁₆H₃₂CO₂H is C18 diacid;
K is 1-isomer of lysine; and
—OH designates the C-terminal amino acid has a terminal carboxylic acid.

The compound of formula (II-i) is the active PTH of the compound of formula (II-i).

The compound of formula (II-i) release a compound of formula (II-ii′)

(II-ii′)
SVSEIQLMHNLGKHLNSMERVEWLRKKLQDVHK(γE-
(miniPEG)₂-γE-COC₁₆H₃₂CO₂H)-OH,

wherein
k is d-Lys;
γE is the 1-isomer of gamma, glutamic acid;
(miniPEG)₂ is COCH₂OCH₂CH₂OCH₂CH₂NH;
COC₁₆H₃₂CO₂H is C18 diacid;
K is 1-isomer of lysine; and
—OH designates the C-terminal amino acid has a terminal carboxylic acid.

The compound of formula (II-ii′) is the active PTH of the compound of formula (II-ii′).

In certain embodiments the initial dose of a compound of formula (II-i) or (II-i′) administered in step (c) is a dose ranging from 300 μg/week to 500 μg/week.

In certain embodiments the second PTH compound is a compound of formula (II-i) and the second average interval is approx. one week. In certain embodiments the second PTH compound is a compound of formula (II-i) and the second average interval is one week. In certain embodiments the second PTH compound is a compound of formula (II-i), the second average interval is one week and administration is via subcutaneous injection. In certain embodiments the second PTH compound is a compound of formula (II-i), the second average interval is one week and administration is via subcutaneous injection with a pen injector.

In certain embodiments the second PTH compound is a compound of formula (II-i′) and the second average interval is approx. one week. In certain embodiments the second PTH compound is a compound of formula (II-i′) and the second average interval is one week. In certain embodiments the second PTH compound is a compound of formula (II-i′), the second average interval is one week and administration is via subcutaneous injection. In certain embodiments the second PTH compound is a compound of formula (II-i′), the second average interval is one week and administration is via subcutaneous injection with a pen injector.

In certain embodiments the second PTH compound is a compound of formula (II-i) and the second interval is approx. one week. In certain embodiments the second PTH compound is a compound of formula (II-i) and the second interval is one week. In certain embodiments the second PTH compound is a compound of formula (II-i), the second interval is one week and administration is via subcutaneous injection. In certain embodiments the second PTH compound is a compound of formula (II-i), the second interval is one week and administration is via subcutaneous injection with a pen injector.

In certain embodiments the second PTH compound is a compound of formula (II-i′) and the second interval is approx. one week. In certain embodiments the second PTH compound is a compound of formula (II-i′) and the second interval is one week. In certain embodiments the second PTH compound is a compound of formula (II-i′), the second interval is one week and administration is via subcutaneous injection. In certain embodiments the second PTH compound is a compound of formula (II-i′), the second interval is one week and administration is via subcutaneous injection with a pen injector.

In certain embodiments the first PTH compound is of formula (I), the first average interval is approx. one day, the second PTH compound is of formula (II-i) and the second average interval is approx. one week. In certain embodiments the first PTH compound is of formula (I), the first average interval is one day, the second PTH compound is of formula (II-i) and the second average interval is one week. In certain embodiments the first PTH compound is of formula (I), the first interval is approx. one day, the second PTH compound is of formula (II-i) and the second interval is approx. one week. In certain embodiments the first PTH compound is of formula (I), the first interval is one day, the second PTH compound is of formula (II-i) and the second interval is one week. In certain embodiments the first PTH compound is of formula (I) with m and p independently being an integer ranging from approx. 450 to 500, the first interval is one day, the second PTH compound is of formula (II-i) and the second interval is one week. In certain embodiments the first PTH compound is of formula (I) with m and p independently being an integer ranging from 450 to 500, the first interval is one day, the second PTH compound is of formula (II-i) and the second interval is one week.

In certain embodiments the first PTH compound is of formula (I), the first average interval is approx. one day, the second PTH compound is of formula (II-i), the second average interval is approx. one week and both the first and the second PTH compound are administered via subcutaneous injection. In certain embodiments the first PTH compound is of formula (I), the first average interval is one day, the second PTH compound is of formula (II-i), the second average interval is one week and both the first and the second PTH compound are administered via subcutaneous injection. In certain embodiments the first PTH compound is of formula (I), the first interval is approx. one day, the second PTH compound is of formula (II-i), the second interval is approx. one week and both the first and the second PTH compound are administered via subcutaneous injection. In certain embodiments the first PTH compound is of formula (I), the first interval is one day, the second PTH compound is of formula (II-i), the second interval is one week and both the first and the second PTH compound are administered via subcutaneous injection. In certain embodiments the first PTH compound is of formula (I) with m and p independently being an integer ranging from approx. 450 to 500, the first interval is one day, the second PTH compound is of formula (II-i), the second interval is one week and both the first and the second PTH compound are administered via subcutaneous injection. In certain embodiments the first PTH compound is of formula (I) with m and p independently being an integer ranging from 450 to 500, the first interval is one day, the second PTH compound is of formula (II-i), the second interval is one week and both the first and the second PTH compound are administered via subcutaneous injection.

In certain embodiments the first PTH compound is of formula (I), the first average interval is approx. one day, the second PTH compound is of formula (II-i′) and the second average interval is approx. one week. In certain embodiments the first PTH compound is of formula (I), the first average interval is one day, the second PTH compound is of formula (II-i′) and the second average interval is one week. In certain embodiments the first PTH compound is of formula (I), the first interval is approx. one day, the second PTH compound is of formula (II-i′) and the second interval is approx. one week. In certain embodiments the first PTH compound is of formula (I), the first interval is one day, the second PTH compound is of formula (II-i′) and the second interval is one week. In certain embodiments the first PTH compound is of formula (I) with m and p independently being an integer ranging from approx. 450 to 500, the first interval is one day, the second PTH compound is of formula (II-i′) and the second interval is one week. In certain embodiments the first PTH compound is of formula (I) with m and p independently being an integer ranging from 450 to 500, the first is one day, the second PTH compound is of formula (II-i′) and the second interval is one week.

In certain embodiments the first PTH compound is of formula (I), the first average interval is approx. one day, the second PTH compound is of formula (II-i′), the second average interval is approx. one week and both the first and the second PTH compound are administered via subcutaneous injection. In certain embodiments the first PTH compound is of formula (I), the first average interval is one day, the second PTH compound is of formula (II-i′), the second average interval is one week and both the first and the second PTH compound are administered via subcutaneous injection. In certain embodiments the first PTH compound is of formula (I), the first interval is approx. one day, the second PTH compound is of formula (II-i′), the second interval is approx. one week and both the first and the second PTH compound are administered via subcutaneous injection. In certain embodiments the first PTH compound is of formula (I), the first interval is one day, the second PTH compound is of formula (II-i′), the second interval is one week and both the first and the second PTH compound are administered via subcutaneous injection. In certain embodiments the first PTH compound is of formula (I) with m and p independently being an integer ranging from approx. 450 to 500, the first interval is one day, the second PTH compound is of formula (II-i′), the second interval is one week and both the first and the second PTH compound are administered via subcutaneous injection. In certain embodiments the first PTH compound is of formula (I) with m and p independently being an integer ranging from 450 to 500, the first interval is one day, the second PTH compound is of formula (II-i′), the second interval is one week and both the first and the second PTH compound are administered via subcutaneous injection.

In certain embodiments the first PTH compound is PTH 1-34, the first average interval is approx. eight hours, the second PTH compound is of formula (II-i) and the second average interval is approx. one week. In certain embodiments the first PTH compound is PTH 1-34, the first average interval is eight hours, the second PTH compound is of formula (II-i) and the second average interval is one week. In certain embodiments the first PTH compound is PTH 1-34, the first interval is approx. eight hours, the second PTH compound is of formula (II-i) and the second interval is approx. one week. In certain embodiments the first PTH compound is PTH 1-34, the first interval is eight hours, the second PTH compound is of formula (II-i) and the second interval is one week. In certain embodiments the first PTH compound is PTH 1-34, the first interval is eight hours, the second PTH compound is of formula (II-i) and the second interval is one week. In certain embodiments the first PTH compound is PTH 1-34, the first interval is eight hours, the second PTH compound is of formula (II-i) and the second interval is one week.

In certain embodiments the first PTH compound is PTH 1-34, the first average interval is approx. eight hours, the second PTH compound is of formula (II-i), the second average interval is approx. one week and both the first and the second PTH compound are administered via subcutaneous injection. In certain embodiments the first PTH compound is PTH 1-34, the first average interval is eight hours, the second PTH compound is of formula (II-i), the second average interval is one week and both the first and the second PTH compound are administered via subcutaneous injection. In certain embodiments the first PTH compound is PTH 1-34, the first interval is approx. eight hours, the second PTH compound is of formula (II-i), the second interval is approx. one week and both the first and the second PTH compound are administered via subcutaneous injection. In certain embodiments the first PTH compound is PTH 1-34, the first interval is eight hours, the second PTH compound is of formula (II-i), the second interval is one week and both the first and the second PTH compound are administered via subcutaneous injection. In certain embodiments the first PTH compound is PTH 1-34, the first interval is eight hours, the second PTH compound is of formula (II-i), the second interval is one week and both the first and the second PTH compound are administered via subcutaneous injection. In certain embodiments the first PTH compound is PTH 1-34, the first interval is eight hours, the second PTH compound is of formula (II-i), the second interval is one week and both the first and the second PTH compound are administered via subcutaneous injection.

In certain embodiments the first PTH compound is PTH 1-34, the first average interval is approx. eight hours, the second PTH compound is of formula (II-i′) and the second average interval is approx. one week. In certain embodiments the first PTH compound is PTH 1-34, the first average interval is eight hours, the second PTH compound is of formula (II-i′) and the second average interval is one week. In certain embodiments the first PTH compound is PTH 1-34, the first interval is approx. eight hours, the second PTH compound is of formula (II-i′) and the second interval is approx. one week. In certain embodiments the first PTH compound is PTH 1-34, the first interval is eight hours, the second PTH compound is of formula (II-i′) and the second interval is one week. In certain embodiments the first PTH compound is PTH 1-34, the first interval is eight hours, the second PTH compound is of formula (II-i′) and the second interval is one week. In certain embodiments the first PTH compound is PTH 1-34, the first is eight hours, the second PTH compound is of formula (II-i′) and the second interval is one week.

In certain embodiments the first PTH compound is PTH 1-34, the first average interval is approx. eight hours, the second PTH compound is of formula (II-i′), the second average interval is approx. one week and both the first and the second PTH compound are administered via subcutaneous injection. In certain embodiments the first PTH compound is PTH 1-34, the first average interval is eight hours, the second PTH compound is of formula (II-i′), the second average interval is one week and both the first and the second PTH compound are administered via subcutaneous injection. In certain embodiments the first PTH compound is PTH 1-34, the first interval is approx. eight hours, the second PTH compound is of formula (II-i′), the second interval is approx. one week and both the first and the second PTH compound are administered via subcutaneous injection. In certain embodiments the first PTH compound is PTH 1-34, the first interval is eight hours, the second PTH compound is of formula (II-i′), the second interval is one week and both the first and the second PTH compound are administered via subcutaneous injection. In certain embodiments the first PTH compound is PTH 1-34 with m and p independently being an integer ranging from approx. 450 to 500, the first interval is eight hours, the second PTH compound is of formula (II-i′), the second interval is one week and both the first and the second PTH compound are administered via subcutaneous injection. In certain embodiments the first PTH compound is PTH 1-34, the first is eight hours, the second PTH compound is of formula (II-i′), the second interval is one week and both the first and the second PTH compound are administered via subcutaneous injection.

In certain embodiments the first PTH compound is PTH 1-34, the first average interval is approx. 12 hours, the second PTH compound is of formula (II-i) and the second average interval is approx. one week. In certain embodiments the first PTH compound is PTH 1-34, the first average interval is 12 hours, the second PTH compound is of formula (II-i) and the second average interval is one week. In certain embodiments the first PTH compound is PTH 1-34, the first interval is approx. 12 hours, the second PTH compound is of formula (II-i) and the second interval is approx. one week. In certain embodiments the first PTH compound is PTH 1-34, the first interval is 12 hours, the second PTH compound is of formula (II-i) and the second interval is one week.

In certain embodiments the first PTH compound is PTH 1-34, the first average interval is approx. 12 hours, the second PTH compound is of formula (II-i), the second average interval is approx. one week and both the first and the second PTH compound are administered via subcutaneous injection. In certain embodiments the first PTH compound is PTH 1-34, the first average interval is 12 hours, the second PTH compound is of formula (II-i), the second average interval is one week and both the first and the second PTH compound are administered via subcutaneous injection. In certain embodiments the first PTH compound is PTH 1-34, the first interval is approx. 12 hours, the second PTH compound is of formula (II-i), the second interval is approx. one week and both the first and the second PTH compound are administered via subcutaneous injection. In certain embodiments the first PTH compound is PTH 1-34, the first interval is 12 hours, the second PTH compound is of formula (II-i), the second interval is one week and both the first and the second PTH compound are administered via subcutaneous injection.

In certain embodiments the first PTH compound is PTH 1-34, the first average interval is approx. 12 hours, the second PTH compound is of formula (II-i′) and the second average interval is approx. one week. In certain embodiments the first PTH compound is PTH 1-34, the first average interval is 12 hours, the second PTH compound is of formula (II-i′) and the second average interval is one week. In certain embodiments the first PTH compound is PTH 1-34, the first interval is approx. 12 hours, the second PTH compound is of formula (II-i′) and the second interval is approx. one week. In certain embodiments the first PTH compound is PTH 1-34, the first interval is 12 hours, the second PTH compound is of formula (II-i′) and the second interval is one week.

In certain embodiments the first PTH compound is PTH 1-34, the first average interval is approx. 12 hours, the second PTH compound is of formula (II-i′), the second average interval is approx. one week and both the first and the second PTH compound are administered via subcutaneous injection. In certain embodiments the first PTH compound is PTH 1-34, the first average interval is 12 hours, the second PTH compound is of formula (II-i′). the second average interval is one week and both the first and the second PTH compound are administered via subcutaneous injection. In certain embodiments the first PTH compound is PTH 1-34, the first interval is approx. 12 hours, the second PTH compound is of formula (II-i′), the second interval is approx. one week and both the first and the second PTH compound are administered via subcutaneous injection. In certain embodiments the first PTH compound is PTH 1-34, the first interval is 12 hours, the second PTH compound is of formula (II-i′). the second interval is one week and both the first and the second PTH compound are administered via subcutaneous injection.

In certain embodiments the first PTH compound is PTH 1-34, the first average interval is approx. one day, the second PTH compound is of formula (II-i) and the second average interval is approx. one week. In certain embodiments the first PTH compound is PTH 1-34, the first average interval is one day, the second PTH compound is of formula (II-i) and the second average interval is one week. In certain embodiments the first PTH compound is PTH 1-34, the first interval is approx. one day, the second PTH compound is of formula (II-i) and the second interval is approx. one week. In certain embodiments the first PTH compound is PTH 1-34, the first interval is one day, the second PTH compound is of formula (II-i) and the second interval is one week.

In certain embodiments the first PTH compound is PTH 1-34, the first average interval is approx. one day, the second PTH compound is of formula (II-i), the second average interval is approx. one week and both the first and the second PTH compound are administered via subcutaneous injection. In certain embodiments the first PTH compound is PTH 1-34, the first average interval is one day, the second PTH compound is of formula (II-i), the second average interval is one week and both the first and the second PTH compound are administered via subcutaneous injection. In certain embodiments the first PTH compound is PTH 1-34, the first interval is approx. one day, the second PTH compound is of formula (II-i). the second interval is approx. one week and both the first and the second PTH compound are administered via subcutaneous injection. In certain embodiments the first PTH compound is PTH 1-34, the first interval is one day, the second PTH compound is of formula (II-i), the second interval is one week and both the first and the second PTH compound are administered via subcutaneous injection.

In certain embodiments the first PTH compound is PTH 1-34, the first average interval is approx. one day, the second PTH compound is of formula (II-i′) and the second average interval is approx. one week. In certain embodiments the first PTH compound is PTH 1-34, the first average interval is one day, the second PTH compound is of formula (II-i′) and the second average interval is one week. In certain embodiments the first PTH compound is PTH 1-34, the first interval is approx. one day, the second PTH compound is of formula (II-i′) and the second interval is approx. one week. In certain embodiments the first PTH compound is PTH 1-34, the first interval is one day, the second PTH compound is of formula (II-i′) and the second interval is one week.

In certain embodiments the first PTH compound is PTH 1-34, the first average interval is approx. one day, the second PTH compound is of formula (II-i′), the second average interval is approx. one week and both the first and the second PTH compound are administered via subcutaneous injection. In certain embodiments the first PTH compound is PTH 1-34, the first average interval is one day, the second PTH compound is of formula (II-i′), the second average interval is one week and both the first and the second PTH compound are administered via subcutaneous injection. In certain embodiments the first PTH compound is PTH 1-34, the first interval is approx. one day, the second PTH compound is of formula (II-i′), the second interval is approx. one week and both the first and the second PTH compound are administered via subcutaneous injection. In certain embodiments the first PTH compound is PTH 1-34, the first interval is one day, the second PTH compound is of formula (II-i′), the second interval is one week and both the first and the second PTH compound are administered via subcutaneous injection.

In certain embodiments the first PTH compound is PTH 1-84, the first average interval is approx. one day, the second PTH compound is of formula (II-i) and the second average interval is approx. one week. In certain embodiments the first PTH compound is PTH 1-84, the first average interval is one day, the second PTH compound is of formula (II-i) and the second average interval is one week. In certain embodiments the first PTH compound is PTH 1-84, the first interval is approx. one day, the second PTH compound is of formula (II-i) and the second interval is approx. one week. In certain embodiments the first PTH compound is PTH 1-84, the first interval is one day, the second PTH compound is of formula (II-i) and the second interval is one week.

In certain embodiments the first PTH compound is PTH 1-84, the first average interval is approx. one day, the second PTH compound is of formula (II-i), the second average interval is approx. one week and both the first and the second PTH compound are administered via subcutaneous injection. In certain embodiments the first PTH compound is PTH 1-84, the first average interval is one day, the second PTH compound is of formula (II-i), the second average interval is one week and both the first and the second PTH compound are administered via subcutaneous injection. In certain embodiments the first PTH compound is PTH 1-84, the first interval is approx. one day, the second PTH compound is of formula (II-i), the second interval is approx. one week and both the first and the second PTH compound are administered via subcutaneous injection. In certain embodiments the first PTH compound is PTH 1-84, the first interval is one day, the second PTH compound is of formula (II-i), the second interval is one week and both the first and the second PTH compound are administered via subcutaneous injection.

In certain embodiments the first PTH compound is PTH 1-84, the first average interval is approx. one day, the second PTH compound is of formula (II-i′) and the second average interval is approx. one week. In certain embodiments the first PTH compound is PTH 1-84, the first average interval is one day, the second PTH compound is of formula (II-i′) and the second average interval is one week. In certain embodiments the first PTH compound is PTH 1-84, the first interval is approx. one day, the second PTH compound is of formula (II-i′) and the second interval is approx. one week. In certain embodiments the first PTH compound is PTH 1-84, the first interval is one day, the second PTH compound is of formula (II-i′) and the second interval is one week.

In certain embodiments the first PTH compound is PTH 1-84, the first average interval is approx. one day, the second PTH compound is of formula (II-i′), the second average interval is approx. one week and both the first and the second PTH compound are administered via subcutaneous injection. In certain embodiments the first PTH compound is PTH 1-84, the first average interval is one day, the second PTH compound is of formula (II-i′), the second average interval is one week and both the first and the second PTH compound are administered via subcutaneous injection. In certain embodiments the first PTH compound is PTH 1-84, the first interval is approx. one day, the second PTH compound is of formula (II-i′), the second interval is approx. one week and both the first and the second PTH compound are administered via subcutaneous injection. In certain embodiments the first PTH compound is PTH 1-84, the first interval is one day, the second PTH compound is of formula (II-i′), the second interval is one week and both the first and the second PTH compound are administered via subcutaneous injection.

In certain embodiments the first PTH compound is AZP-3601, the first average interval is approx. one day, the second PTH compound is of formula (II-i) and the second average interval is approx. one week. In certain embodiments the first PTH compound is AZP-3601, the first average interval is one day, the second PTH compound is of formula (II-i) and the second average interval is one week. In certain embodiments the first PTH compound is AZP-3601, the first interval is approx. one day, the second PTH compound is of formula (II-i) and the second interval is approx. one week. In certain embodiments the first PTH compound is AZP-3601, the first interval is one day, the second PTH compound is of formula (II-i) and the second interval is one week.

In certain embodiments the first PTH compound is AZP-3601, the first average interval is approx. one day, the second PTH compound is of formula (II-i), the second average interval is approx. one week and both the first and the second PTH compound are administered via subcutaneous injection. In certain embodiments the first PTH compound is AZP-3601, the first average interval is one day, the second PTH compound is of formula (II-i), the second average interval is one week and both the first and the second PTH compound are administered via subcutaneous injection. In certain embodiments the first PTH compound is AZP-3601, the first interval is approx. one day, the second PTH compound is of formula (II-i), the second interval is approx. one week and both the first and the second PTH compound are administered via subcutaneous injection. In certain embodiments the first PTH compound is AZP-3601, the first interval is one day, the second PTH compound is of formula (II-i), the second interval is one week and both the first and the second PTH compound are administered via subcutaneous injection.

In certain embodiments the first PTH compound is AZP-3601, the first average interval is approx. one day, the second PTH compound is of formula (II-i′) and the second average interval is approx. one week. In certain embodiments the first PTH compound is AZP-3601, the first average interval is one day, the second PTH compound is of formula (II-i′) and the second average interval is one week. In certain embodiments the first PTH compound is AZP-3601, the first interval is approx. one day, the second PTH compound is of formula (II-i′) and the second interval is approx. one week. In certain embodiments the first PTH compound is AZP-3601, the first interval is one day, the second PTH compound is of formula (II-i′) and the second interval is one week.

In certain embodiments the first PTH compound is AZP-3601, the first average interval is approx. one day, the second PTH compound is of formula (II-i′), the second average interval is approx. one week and both the first and the second PTH compound are administered via subcutaneous injection. In certain embodiments the first PTH compound is AZP-3601, the first average interval is one day, the second PTH compound is of formula (II-i′), the second average interval is one week and both the first and the second PTH compound are administered via subcutaneous injection. In certain embodiments the first PTH compound is AZP-3601, the first interval is approx. one day, the second PTH compound is of formula (II-i′), the second interval is approx. one week and both the first and the second PTH compound are administered via subcutaneous injection. In certain embodiments the first PTH compound is AZP-3601, the first interval is one day, the second PTH compound is of formula (II-i′), the second interval is one week and both the first and the second PTH compound are administered via subcutaneous injection In certain embodiments step (c) is administering the compound of formula (II-a) on multiple occasions with a second average interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-a) with each n independently being an integer ranging from 200 to 250 on multiple occasions with a second average interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-a) via subcutaneous injection on multiple occasions with a second average interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-a) with each n independently being an integer ranging from 200 to 250 via subcutaneous injection on multiple occasions with a second average interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-a) on multiple occasions with a second interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-a) with each n independently being an integer ranging from 200 to 250 on multiple occasions with a second interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-a) via subcutaneous injection on multiple occasions with a second interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-a) with each n independently being an integer ranging from 200 to 250 via subcutaneous injection on multiple occasions with a second interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-a′) on multiple occasions with a second average interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-a′) with each n independently being an integer ranging from 200 to 250 on multiple occasions with a second average interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-a′) via subcutaneous injection on multiple occasions with a second average interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-a′) with each n independently being an integer ranging from 200 to 250 via subcutaneous injection on multiple occasions with a second average interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-a′) on multiple occasions with a second interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-a′) with each n independently being an integer ranging from 200 to 250 on multiple occasions with a second interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-a′) via subcutaneous injection on multiple occasions with a second interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-a′) with each n independently being an integer ranging from 200 to 250 via subcutaneous injection on multiple occasions with a second interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-b) on multiple occasions with a second average interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-b) with each n independently being an integer ranging from 200 to 250 on multiple occasions with a second average interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-b) via subcutaneous injection on multiple occasions with a second average interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-b) with each n independently being an integer ranging from 200 to 250 via subcutaneous injection on multiple occasions with a second average interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-b) on multiple occasions with a second interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-b) with each n independently being an integer ranging from 200 to 250 on multiple occasions with a second interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-b) via subcutaneous injection on multiple occasions with a second interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-b) with each n independently being an integer ranging from 200 to 250 via subcutaneous injection on multiple occasions with a second interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-b′) on multiple occasions with a second average interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-b′) with each n independently being an integer ranging from 200 to 250 on multiple occasions with a second average interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-b′) via subcutaneous injection on multiple occasions with a second average interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-b′) with each n independently being an integer ranging from 200 to 250 via subcutaneous injection on multiple occasions with a second average interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-b′) on multiple occasions with a second interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-b′) with each n independently being an integer ranging from 200 to 250 on multiple occasions with a second interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-b′) via subcutaneous injection on multiple occasions with a second interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-b′) with each n independently being an integer ranging from 200 to 250 via subcutaneous injection on multiple occasions with a second interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-i) on multiple occasions with a second average interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-i) via subcutaneous injection on multiple occasions with a second average interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-i) on multiple occasions with a second interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-i) via subcutaneous injection on multiple occasions with a second interval between administrations of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-i′) on multiple occasions with a second average interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-i′) via subcutaneous injection on multiple occasions with a second average interval of one week to the patient starting at the initial dose determined in step (b) with adjustment of the dose, if needed, until the patient's serum calcium remains within normal range in the absence of active vitamin D and calcium supplement.

In certain embodiments step (c) is administering the compound of formula (II-i′) on multiple occasions with a second interval between administrations of one week to the patient starting at the initial dose determined in step (b) with adjustment of the dose if needed, until the patient's serum calcium remains within normal range in the absence of active vitamin D and calcium supplement.

In certain embodiments step (c) is administering the compound of formula (II-i′) via subcutaneous injection on multiple occasions with a second interval between administrations of one week to the patient starting at the initial dose determined in step (b) with adjustment of the dose, if needed, until the patient's serum calcium remains within normal range in the absence of active vitamin D and calcium supplement.

In certain embodiments the population of patients of the tenth aspect comprises adult or pediatric patients. In certain embodiments the population of patients of the tenth aspect comprises adult patients. In certain embodiments the population of patients of the tenth aspect comprises pediatric patients.

In certain embodiments the population of patients of the tenths aspect comprises patients having chronic hypoparathyroidism due to surgery, a genetic cause, immune system-related damage of the parathyroid glands or idiopathic hypoparathyroidism.

In the tenths aspect in certain embodiments the dose of the first PTH compound in step (a) is titrated up in some patients, titrated down in some patients, and unchanged in some patients.

In the tenth aspect in certain embodiments the dose of the second PTH compound is titrated up from the initial dose on one or more subsequent administrations in some patients, titrated down from the initial dose on one or more subsequent administrations in some patients, and unchanged from the initial dose in some patients.

Embodiments for all other features of the tenth aspect are as described elsewhere herein.

In certain embodiments, the second PTH compound is a conjugate comprising an acetylated PTH moiety. Suitable conjugates of acetylated PTH are disclosed in WO2021/242756, hereby incorporated by reference in its entirety. In certain embodiments, the first PTH compound may be an acetylated PTH.

In certain embodiments, the PTH conjugate comprises a PTH moiety and a C₁₆-C₃₀ fatty acid or C₁₆-C₃₀ diacid covalently linked to the side chain of an amino acid of the PTH moiety, optionally via a spacer, optionally wherein the acylated amino acid is selected from the group consisting of Lys, dLys, ornithine, Cys and homocysteine.

In certain embodiments, said PTH moiety comprises the sequence SVSEIQLMHNLGKHLNSMERVEWLRKKLQDVH (SEQ ID NO:53 herein), or a PTH moiety comprising the sequence of SEQ ID NO:53 that differs by 1, 2 or 3 amino acid substitutions.

In certain embodiments the PTH moiety comprises an amino acid sequence selected from the group consisting of:

(SEQ ID NO: 123)
SVSEIQLMHX₁₀LGX₁₃HLX₁₆SX₁₈ERVEWLRX₂₆X₂₇LQDX₃₁H-Z,;
(SEQ ID NO: 124)
SVSEIQLMHX₁₀LX₁₂KHLX₅₆X₁₇X₁₈ERVEWLRKKLQDVH-Z;;
(SEQ ID NO: 125)
SVSEIQLMHX₁₀LGKHLX₁₆SX₁₈ERVEWLRKKLQDVH-Z
and
(SEQ ID NO: 126)
SVSEIQLMHNLGKHLNSMERVEWLRKKLQDVH-Z;

wherein Z is X₃₃, X₃₃F, X₃₃FX₃₅, X₃₃FVX₃₅, X₃₃FVAX₃₅, X₃₃FVALX₃₅ (SEQ ID NO:127), X₃₃FVALGX₃₅ (SEQ ID NO: 128), X₅₃X₃₅, X₅₃FX₃₅, X₅₃FVX₃₅, X₅₃FVAX₃₅, X₅₃FVALX₃₅ (SEQ ID NO:129), or X₅₃FVALGX₃₅ (SEQ ID NO:130), optionally wherein Z is X₃₃, X₅₃X₃₅, X₅₃FX₃₅, X₅₃FVX₃₅, X₅₃FVAX₃₅, X₅₃FVALX₃₅ (SEQ ID NO:129), or X₅₃FVALGX₃₅ (SEQ ID NO:130);
X₁₀ and X₁₆ are independently Asp, Gln or Asn;
X₁₂ is Gly or Aib;
X₅₆ is amino isobutyric acid (Aib) or Asn
X₁₇ is amino isobutyric acid (Aib) or Ser;
X₁₈ is Met, Met(O), Leu, or Nleu;
X₁₃, X₂₆, and X₂₇ are independently selected from the group consisting of Arg, Glu, Asp and Lys;
X₃₁ is Gly or Val;
X₃₃ and X₃₅ each comprise an acylated amino acid comprising a C₁₆-C₃₀ fatty acid or C₁₆-C₃₀ diacid covalently linked to the side chain of the amino acid, optionally via a spacer, optionally wherein the acylated amino acid is selected from the group consisting of Lys, dLys, ornithine, Cys and homocysteine;
X₅₃ is Gln or Asn, optionally with the proviso that no more than one of X₁₂, X₁₆ and X₁₇ is Aib, and optionally wherein the C-terminal amino acid is modified to replace the carboxy terminus with an amide.

In certain embodiments Z is X₃₃, X₅₃X₃₅, X₅₃FX₃₅, X₅₃FVX₃₅, X₅₃FV AX₃₅, X₅₃FVALX₃₅ (SEQ ID NO:129), or X₅₃FVALGX₃₅ (SEQ ID NO:130).

In certain embodiments, the PTH moiety comprises the sequence

(SEQ ID NO: 126)
SVSEIQLMHNLGKHLNSMERVEWLRKKLQDVH-Z,

wherein
Z is X₃₃F, X₅₃X₃₅, X₅₃FX₃₅, or X₃₃;
X₃₃ and X₃₅ are each independently an amino acid comprising a C₁₆-C₃₀ fatty acid or C₁₆-C₃₀ diacid covalently linked to the acid side chain of the amino acid, optionally via a spacer; and X₅₃ is Asn.

In certain embodiments, the PTH moiety comprises the sequence of

(SEQ ID NO: 131)
SVSEIQLMHNLGKHLNSMERVEWLRKKLQDVHX₃₃
or
(SEQ ID NO: 132)
SVSEIQLMHNLGKHLNSMERVEWLRKKLQDVHNFX₃₅

wherein X₃₃ and X₃₅ are each independently an amino acid comprising a C₁₆-C₂₂ fatty acid or C₁₆-C₂₂ diacid covalently linked to the side chain of the amino acid, optionally via a spacer.

In certain embodiments the second PTH compound comprises a peptide moiety and a self-cleaving dipeptide covalently bound to said PTH moiety via an amide bond, wherein said self-cleaving dipeptide comprises the structure A-B where A is an amino acid; and B is an N-alkylated amino acid.

In certain embodiments, the second PTH compound is a conjugate comprising a PTH moiety and a self-cleaving dipeptide covalently bound to said PTH moiety via an amide bond, said PTH moiety comprising an amino acid sequence selected from the group consisting of SVSEIQLMHNLGX₁₃HLNSMERVEWLRX₂₆X₂₇LQDX₃₁H—Z, (SEQ ID NO:133); SVSEIQLMHXioLGKHLX₁₆SXisERVEWLRKKLQDVH-Z (SEQ ID NO:134); SVSEIQLMHNLGKHLNSMERVEWLRKKLQDVH-Z (SEQ ID NO:126), and a sequence that differs from the SEQ ID NO: 7 by 1, 2 or 3 amino acid substitutions;
wherein
Z is X₃₃F, X₅₃X₃₅, X₅₃FX₃₅, or X₃₃;
X₁₀ and X₁₆ are independently Asp, Gln or Asn;
X₁₈ is Met, Met(O), Leu, or Nleu;
X₁₃, X₂₆, and X₂₇ are independently selected from the group consisting of Arg, Glu, Asp and Lys;
X₃₁ is Gly or Val;
X₃₃ and X₃₅ each comprise an acylated amino acid;
X₅₃ is Gln or Asn;
and said self-cleaving dipeptide comprising the general structure A-B; wherein A is an amino acid or an acylated amino acid; B is an N-alkylated amino acid; wherein said acylated amino acid of each of X₃₃, X₃₅ and A is independently selected from an amino acid comprising a C₁₆-C₃₀ fatty acid or C₁₆-C₃₀ diacid covalently linked to the amino acid side chain, optionally via a spacer, and said self-cleaving dipeptide is linked to said PTH moiety through formation of an amide bond between B and the N-terminal alpha amine of said PTH moiety, further wherein said optional spacer of each of X₃₃, X₃₅ and A comprises one or more linker moieties independently selected from the group consisting of a gamma glutamic acid, and COCH₂(OCH₂CH₂)ₖNH, wherein k is an integer selected from the range of 1 to 8; with the proviso that when A is a non-acylated amino acid, then A is an amino acid in the D-stereochemical configuration.

In certain embodiments A is selected from the group consisting of Lys, dLys, acylated-Lys and acylated-dLys wherein said self-cleaving dipeptide is covalently linked to the N-terminal alpha amine of the PTH peptide.

In certain embodiments the acylated amino acid of each of X₃₃, X₃₅ and A comprises a C₁₆-C₃₀ fatty acid or C₁₆-C₃₀ diacid covalently linked to the amino acid side chain via a spacer, wherein the spacer of each of X₃₃, X₃₅ and A is independently selected from the group comprising a gamma glutamic acid-gamma glutamic acid dipeptide, (Xaa)-[COCH₂(OCH₂CH₂)ₖNH]_{q}-gamma glutamic acid and a gamma glutamic acid-[COCH₂(OCH₂CH₂)ₖNH]_{q}-gamma glutamic acid, wherein Xaa is selected from the group consisting of Arg, Tyr(OPO₃H₂) and hCys(SO₃H); k is an integer selected from the range of 1 to 8; and q is an integer selected from the range of 1 to 8, optionally wherein k is 2 and q is selected from the range of 1 to 4.

In certain embodiments the acylated amino acid of A, X₃₃ and X₃₅ is independently selected from the group consisting of cysteine, homocysteine, ornithine lysine and d-lysine, wherein the side chain of said cysteine, homocysteine, ornithine lysine or d-lysine is covalently linked to a C₁₆-C₂₂ fatty acid or C₁₆-C₂₂ diacid, optionally through a spacer, wherein the optional spacer of each of A, X₃₃ and X₃₅ comprises a gamma glutamic acid linkage.

In certain embodiments, the acylated amino acid of A, X₃₃ and X₃₅ is each independently selected from the group consisting of lysine and d-lysine, wherein the side chain of said lysine or d-lysine is covalently linked to a C₁₆-C₂₂ fatty acid or C₁₆-C₂₂ diacid, optionally through a spacer comprising a gamma glutamic acid linkage.

In certain embodiments the acylated amino acid of A is d- lysine and X₃₃ and X₃₅ are each lysine.

In certain embodiments the spacer of A, X₃₃ and X₃₅ is each independently selected from compounds comprising the structure: gamma glutamic acid-[COCH₂(OCH₂CH₂)ₖNH]_{q}-gamma glutamic acid, wherein k is an integer selected from 2, 4 or 8 and q is an integer selected from 1, 2, 4 or 8.

In certain embodiments, k is 2 and q is 2 or 4.

In certain embodiments A-B comprises the structure:

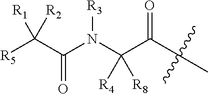

wherein
R₁ comprises a side chain selected from the group consisting of C₁-C₈ alkyl, (C₁-C₄ alkyl)OH, (C₁-C₄ alkyl)SH, (C₁-C₄ alkyl)COOH, and (C₁-C₄ alkyl)NH₂, optionally wherein a C₁₆-C₃₀ fatty acid or C₁₆-C₃₀ diacid is covalently linked to said side chain, optionally via a spacer selected from the group consisting of a gamma glutamic acid, a gamma glutamic acid-gamma glutamic acid dipeptide, and a gamma glutamic acid-[COCH₂(OCH₂CH₂)ₖNH]_{q}-gamma glutamic acid, wherein k is an integer selected from the range of 1 to 8; and
q is an integer selected from the range of 1 to 8, optionally wherein k is 2 and q is selected from the range of 1 to 8;
R₂, R₄ and R₅ are independently H, or C₁-C₄ alkyl; R₃ is C₁-C₆ alkyl; and R₅ is NH2.

In certain embodiments the release half-life of A-B is at least about 48 to 168 hours in standard PBS solution under physiological conditions.

In certain embodiments R₁ is (C₁-C₄ alkyl)NH; R₂ and R₈ are each H; R₄ is H, or CH₃; R₃ is CH₃ and R₅ is NH₂.

In certain embodiments R₁ is (C₁-C₄ alkyl)NH-[spacer]-CO(CH₂)₁₄₋₂₀COOH, (C₁-C₄ alkyl)NH—[spacer]-CO(CH₂)₁₄₋₂₀CH₃, (C₁-C₄ alkyl)NH—CO(CH₂)₁₄₋₂₀COOH, or (C₁-C₄ alkyl)NH—CO(CH₂)₁₄₋₂₀CH₃; R₂ and R₈ are each H; R₄ is H, or CH₃; R₃ is CH₃ and R₅ is NH₂, wherein said [spacer] is a linking moiety selected from the group consisting of a gamma glutamic acid, a gamma glutamic acid-gamma glutamic acid dipeptide, and a gamma glutamic acid-[COCH₂(OCH₂CH₂)ₖNH]_{q}-gamma glutamic acid, wherein k is an integer selected from the range of 2 to 4 and q is an integer selected from the range of 1 to 8.

In certain embodiments R₁ is (C₁-C₄ alkyl)NH-[spacer]-CO(CH₂)₁₄₋₂₀COOH, wherein said [spacer] is a linking moiety comprising the structure gamma glutamic acid-[COCH₂(OCH₂CH₂)ₖNH]_{q}-gamma glutamic acid, wherein k is 2 or 4 and q is 1, 2 or 4.

In certain embodiments R₁ is (C₄ alkyl)NH-{gamma glutamic acid-[COCH₂(OCH₂CH₂)₂NH—COCH₂(OCH₂CH₂)₂]NH-gamma glutamic acid}-CO(CH₂)₁₄₋₂₀COOH.

In certain embodiments the first amino acid of the cleavable dipeptide is an amino acid in the D-stereochemical configuration.

In certain embodiments the second PTH compound is a conjugate comprising a PTH moiety and a self-cleaving dipeptide covalently linked to the N-terminal alpha amine of said PTH moiety via an amide bond, wherein said PTH moiety comprises the amino acid sequence of SVSEIQLMHNLGKHLNSMERVEWLRKKLQDVHX₃₃ (SEQ ID NO:131), SVSEIQLMHNLGKHLNSMERVEWLRKKLQDVHNFX₃₅ (SEQ ID NO: 132), or a moiety that differs from SEQ ID NO:131 or SEQ ID NO:132 by 1 or 2 amino acid substitutions
wherein
X₃₃ and X₃₅ are each an amino acid comprising a side chain of (C₁-C₄ alkyl)NH—CO(CH₂)₁₄₋₂₀COOH, (C₁-C₄ alkyl)NH-[spacer]-CO(CH₂)₁₄₋₂₀COOH, (C₁-C₄ alkyl)NH—CO(CH₂)₁₄₋₂₀CH₃ or (C₁-C₄ alkyl)NH-[spacer]-CO(CH₂)₁₄₋₂₀CH₃; and said self-cleaving dipeptide comprises the general structure:

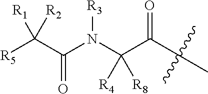

wherein
R₁ is selected from the group consisting of (C₁-C₄ alkyl)NH, (C₁-C₄ alkyl)NH—CO(CH₂)₁₄₋₂₀COOH, (C₁-C₄ alkyl)NH-[spacer]-CO(CH₂)₁₄₋₂₀COOH, (C₁-C₄ alkyl)NH—CO(CH₂)₁₄₋₂₀CH₃ and (C₁-C₄ alkyl)NH-[spacer]-CO(CH₂)₁₄₋₂₀CH₃; R₂ and R₈ are each H; R₄ is H or CH₃; R₃ is C₁-C₃ alkyl and R₅ is NH₂, wherein said spacer of R₁, X₃₃ and X₃₅ is each independently selected from the group consisting of gamma glutamic acid, gamma glutamic acid- gamma glutamic acid dipeptide and gamma glutamic acid- [COCH₂(OCH₂CH₂)ₖNH]_{q}-gamma glutamic acid, wherein k is an integer selected from the range of 2 to 4 and q is an integer selected from the range of 1 to 4.

In certain embodiments the PTH moiety comprises the amino acid sequence of

(SEQ ID NO: 132)
SVSEIQLMHNLGKHLNSMERVEWLRKKLQDVHNFX₃₅,

wherein X₃₅ is an amino acid comprising a side chain of (C₁-C₄ alkyl)NH-{gamma glutamic acid-[COCH₂(OCH₂CH₂)₂NH—COCH₂(OCH₂CH₂)₂]NH-gamma glutamic acid}-CO(CH₂)₁₄₋₂₀COOH; and said self-cleaving dipeptide comprises the general structure:

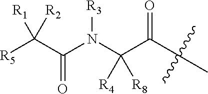

wherein
R₁ is (C₁-C₄ alkyl)NH-{gamma glutamic acid-[COCH₂(OCH₂CH₂)₂NH—COCH₂(OCH₂CH₂)₂]NH-gamma glutamic acid}-CO(CH₂)₁₄₋₂₀COOH; R₂ and R₈ are each H; R₃ is CH₃; and R₅ is NH₂.

In certain embodiments the PTH moiety comprises the amino acid sequence of

(SEQ ID NO: 131)
SVSEIQLMHNLGKHLNSMERVEWLRKKLQDVHX₃₃,

wherein X₃₃ is an amino acid comprising a side chain of (C₁-C₄ alkyl)NH-{gamma glutamic acid-[COCH₂(OCH₂CH₂)k-NH]_{q}-gamma glutamic acid}-CO(CH₂)₁₄₋₂₀COOH; and said self-cleaving dipeptide comprises the general structure:

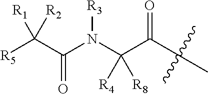

wherein
R₁ is (C₁-C₄ alkyl)NH-{gamma glutamic acid-[COCH₂(OCH₂CH₂)ₖNH]_{q}—gamma glutamic acid}-CO(CH₂)₁₆₋₁₈COOH; R₂ and R₈ are each H; R₃ is CH₃; R₅ is NH₂;
q is 2 or 4 and k is 2.

In certain embodiments X₃₃ is an amino acid comprising a side chain of (C₄alkyl)NH-{gamma glutamic acid-[COCH₂(OCH₂CH₂)₂NH—COCH₂(OCH₂CH₂)₂]NH-gamma glutamic acid}-CO(CH₂)i₆COOH; and R₁ is (C₄ alkyl)NH-{gamma glutamic acid-[COCH₂(OCH₂CH₂)₂NH—COCH₂(OCH₂CH₂)₂]NH-gamma glutamic acid}-CO(CH₂)₁₆COOH.

In certain embodiments, the first amino acid of the self-cleaving dipeptide is in the D-stereochemical configuration.
[01] A first and a second PTH compound for use in a method of treating chronic hypoparathyroidism, the method comprising:
   (a) administering to a patient having chronic hypoparathyroidism the first PTH compound on multiple occasions with a first average interval between administrations, adjusting dose if needed, to determine for the patient a dose at which the patient's serum calcium level remains within normal range in the absence of active vitamin D and calcium supplement;
   (b) using the dose determined in step (a) to determine an initial dose for the second PTH compound to be administered on multiple occasions to the patient, with a second average interval, longer than the first average interval; and
   (c) administering the second PTH compound on multiple occasions with the second average interval between administrations to the patient starting at the initial dose determined in step (b) with adjustment of the dose, if needed, until the patient's serum calcium remains within normal range in the absence of active vitamin D and calcium supplement.
[02] The first and second PTH compound for use of paragraph 1, wherein the patient has chronic hypoparathyroidism due to surgery, a genetic cause, immune system-related damage of the parathyroid glands or the hypoparathyroidism is idiopathic.
[03] The first and second PTH compound for use of paragraph 1 or 2, wherein the first PTH compound and the second PTH compound are administered by subcutaneous injection.
[04] The first and second PTH compound for use of any one of paragraphs 1 to 3, wherein the first average interval ranges from 6 hours to one week.
[05] The first and second PTH compound for use of any one of paragraphs 1 to 4, wherein the dose of the first PTH compound is increased between at least two consecutive administrations in response to hypocalcemia.
[06] The first and second PTH compound for use of any one of paragraphs 1 to 5, wherein the dose of the first PTH compound is decreased between at least two consecutive administrations responsive to hypercalcemia.
[07] The first and second PTH compound for use of any one of paragraphs 1 to 6, wherein the first PTH compound is of formula (I)

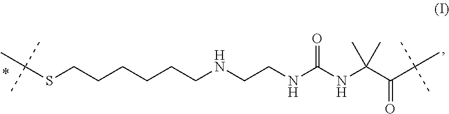

wherein
the unmarked dashed line indicates the attachment to the nitrogen of the N-terminal amine group of a PTH moiety of SEQ ID NO:51; and
the dashed line marked with the asterisk indicates attachment to a moiety

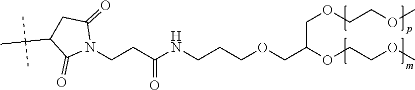

wherein
m and p are independently an integer ranging from 400 to 500.
[08] The first and second PTH compound for use of any one of paragraphs 1 to 6, wherein the first PTH compound is AZP-3601.
[09] The first and second PTH compound for use of any one of paragraphs 1 to 6, wherein the first PTH compound is PTH 1-84.
[010] The first and second PTH compound for use of any one of paragraphs 1 to 9, wherein the first average interval between administrations is one day.
[011] The first and second PTH compound for use of any one of paragraphs 1 to 6, wherein the first PTH compound is PTH 1-34.
[012] The first and second PTH compound for use of paragraphs 11, wherein the first average interval between administrations is 8 hours.
[013] The first and second PTH compound for use of any one of paragraphs 1 to 12, wherein all intervals between administrations in step (a) have the same length.
[014] The first and second PTH compound for use of any one of paragraphs 1 to 13, wherein the first treatment period, which is the period of time from the first to the last occasion of administration of the first PTH compound in step (a), lasts from one week to 20 years.
[015] The first and second PTH compound for use of paragraphs 14, wherein the first treatment period lasts at least until the patient has serum calcium levels that are within the normal range and has discontinued active vitamin D and calcium supplement.
[016] The first and second PTH compound for use of any one of paragraphs 1 to 15, wherein the first PTH compound is the only drug comprising PTH or a PTH moiety administered to the patient during step (a).
[017] The first and second PTH compound for use of any one of paragraphs 1 to 16, wherein no dose adjustment occurred for at least the last interval of the first treatment period before administration of the first dose of the second PTH compound.
[018] The first and second PTH compound for use of any one of paragraphs 1 to 17, wherein the active PTH of the first and the second PTH compound are the same and the initial dose of the second PTH compound is D₂±25% and wherein D₂ is calculated using the equation

$${D}_{2} = {D}_{1} \times \frac{{T}_{2}}{{T}_{1}}$$

wherein
D₁ is the dose of the first PTH compound at which the patient's serum calcium level remains within normal range in the absence of active vitamin D and calcium supplement;
T₁ is the first interval between administrations; and
T₂ is the second interval between administrations.

The first and second PTH compound for use of any one of paragraphs 1 to 17, wherein the active PTH of the first and the second PTH compound are different and the initial dose of the second PTH compound is calculated by first calculating a correction factor CF using the following equation

$$CF = \frac{{D}_{supp 2}}{{D}_{supp 1}} \times \frac{{T}_{supp 1}}{{T}_{supp 2}}$$

wherein
Dₛᵤₚₚ₁ is the dose of the first PTH compound that fully suppresses secretion of endogenous PTH 1-84 in healthy volunteers;
Dₛᵤₚₚ₂ is the dose of the second PTH compound that fully suppresses secretion of endogenous PTH 1-84 in healthy volunteers;
   Tₛᵤₚₚ₁ is the length of the first interval between administrations to the healthy volunteers; and
   Tₛᵤₚₚ₂ is the length of the second interval between administrations to the healthy volunteers;
   followed by calculating D₂ using the following equation

$${D}_{2} = {D}_{1} \times CF \times \frac{{T}_{2}}{{T}_{1}}$$

wherein
D₁ is the dose of the first PTH compound at which the patient's serum calcium level remains within normal range in the absence of active vitamin D and calcium supplement;
T₁ is the first interval between administrations;
T₂ is the second interval between administrations;
and wherein the initial dose of the second PTH compound is D₂+25%.
[020] The first and the second PTH compound for use of any one of paragraphs 1 to 19, wherein the time between the last dose of the first PTH compound and the first dose of the second PTH compound corresponds to the average interval between two administrations of the first PTH compound.
[021] The first and the second PTH compound for use of any one of paragraphs 1 to 20, wherein the second PTH compound is the only drug comprising PTH or a PTH moiety administered to the patient during step (c).
[022] The first and the second PTH compound for use of any one of paragraphs 1 to 21, wherein the second average interval is at least 2-fold longer than the first average interval.
[023] The first and the second PTH compound for use of any one of paragraphs 1 to 22, wherein the second PTH compound is a compound of formula (II-a)

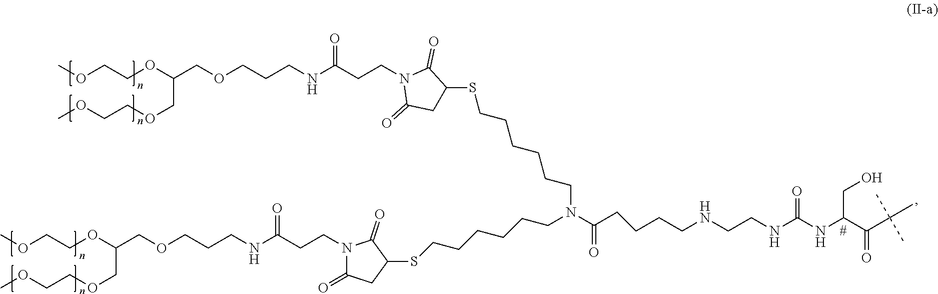

wherein
the dashed line indicates attachment to the nitrogen of the N-terminal amine group of a PTH moiety having the sequence of SEQ ID NO:51; and
each n is independently an integer ranging from 200 to 250.
[024] The first and the second PTH compound for use of any one of paragraphs 1 to 23, wherein the second PTH compound is a compound of formula of formula (II-i)

(II-i)
k(γE-(miniPEG)₂-γE-COC₁₆H₃₂CO₂H)(N-Me)GSVSEIQL
MHNLGKHLNSMERVEWLRKKLQDVHK(γE-(miniPEG)₂-γE-
COC₁₆H₃₂CO₂H)-OH,

wherein
k is d-Lys;
γE is the 1-isomer of gamma, glutamic acid;
miniPEG is COCH₂OCH₂CH₂OCH₂CH₂NH;
COC₁₆H₃₂CO₂H is C18 diacid;
(N-Me)G is sarcosine;
K is 1-isomer of lysine; and
—OH designates the C-terminal amino acid has a terminal carboxylic acid.
[025] The first and the second PTH compound for use any one of paragraphs 1 to 24, wherein the second average interval between administrations is one week.
[026] The first and the second PTH compound for use any one of paragraphs 1 25 23, wherein the second treatment period lasts for as long as the patient benefits from administration of the second PTH compound.

### Materials

Compound 1 has the following structure

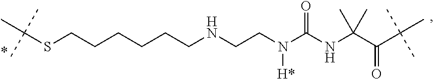

wherein
the unmarked dashed line indicates the attachment to the nitrogen of the N-terminal amine group of a PTH moiety of SEQ ID NO:51; and
the dashed line marked with the asterisk indicates attachment to a moiety

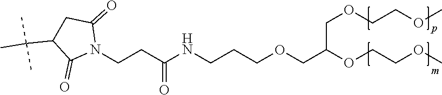

wherein
m and p are independently an integer ranging from 450 to 500.

Compound 1 can be manufactured as described for compound 18 in WO2017/148883A1. Compound 2 has the following structure

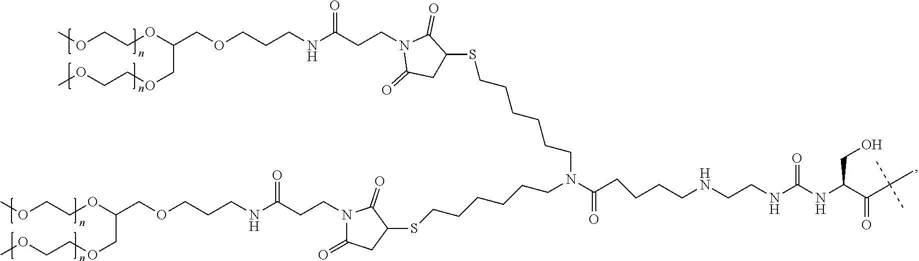

wherein
the dashed line indicates attachment to the nitrogen of the N-terminal amine group of a PTH moiety having the sequence of SEQ ID NO:51; and
each n is independently an integer ranging from 200 to 250.

Compound 2 can be manufactured as described for compound 31 in WO2017/148883A1.

Compound 3 has the following structure

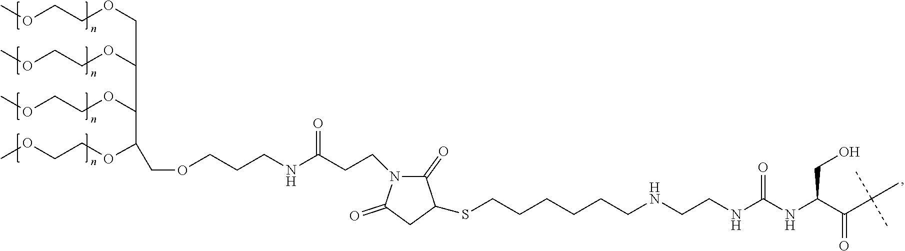

wherein
the dashed line indicates attachment to the nitrogen of the N-terminal amine group of a PTH moiety having the sequence of SEQ ID NO:51; and
each n is independently an integer ranging from 200 to 250.

Compound 3 can be manufactured as compound 3 with the difference that instead of the PEG reagent SUNBRIGHT GL2-200MA the reagent used is SUNBRIGHT XY4-400MA having the following structure

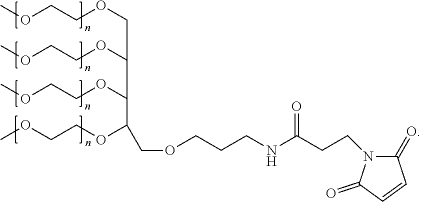

Compound 4 corresponds to SEQ ID NO:87 of WO2021/242756A2 and can be manufactured as described in example 2, page 77/78 therein.

Compound 5 is a polypeptide having the sequence AVAEIQLMHQRAKWIQDARR RAFLHKLIAEIHTAEI, which is SEQ ID NO:122 herein, and which can be manufactured as described for SEQ ID NO:4 of U.S. Pat. No. 9,492,508, example 1.

### Example 1

### Phase 3 Clinical Study of Compound 1

PaTHway is a phase 3, multicenter (North America and Europe), randomized, double-blind, placebo-controlled, parallel-group, 26-week trial with an open-label extension of 156 weeks that evaluated the efficacy, safety, and tolerability of once-daily Compound 1 as PTH replacement therapy in individuals with hypoparathyroidism. All participants who completed the 26- week blinded treatment period were allowed to enroll in the 156-week open-label extension period. (ClinicalTrials.gov identifier: NCT04701203; EudraCT No.: 2020-003380-26).

Once enrolled, participants were randomized 3:1 into 2 treatment groups: Compound 1 18 μg PTH(1-34)/day or placebo (excipient solution) mimicking 18 μg/day, both co-administered with conventional therapy (active vitamin D and elemental calcium).

Randomization was stratified by etiology of hypoparathyroidism (postsurgical vs. non-surgical). In addition to frequent laboratory visits to measure serum calcium levels, the blinded treatment period included 10 clinic visits over 26 weeks. Once-daily treatment with Compound 1 or placebo was delivered by a modified Ypsomed UnoPen Fix pen injector using 31-gauge, 5 mm pen needles to deliver doses of 6-30 μg/injection in a volume of ≤100 μL to either abdomen or anterior thigh, rotating injection sites.

All participants were initially prescribed Compound 1 18 μg PTH(1-34)/day or a corresponding volume of placebo and were individually and progressively titrated to an optimal dose (allowable range 6-60 μg/day) in increments of 3 μg/day. Titration of study drug and conventional therapy was performed according to a protocol-specified algorithm guided by serum calcium values (Example 2). The algorithm was intended to facilitate independence from conventional therapy by discontinuation of active vitamin D and calcium in response to reestablishment of physiological PTH signaling with Compound 1 treatment. In the setting of normocalcemia, the protocol directs incremental increases in study drug (Compound 1 or placebo) accompanied by stepwise reductions of active vitamin D by 33-50% of the starting dose, followed by reductions of calcium in at least 1500 mg/day increments once vitamin D has been discontinued.

Participants were permitted to take elemental calcium <600 mg/day as a nutritional supplement if required to meet the generally recommended dietary intakes of calcium.

If required for using dose prediction formulas of Example 7 and 8 below, it may be required to report the dose in molar equivalents. It is standard in the art to convert the mass dose to the molar dose. As an example, for PTH(1-34) with a molecular mass of 4117.8 Daltons, the molar dose is 4.37 nmol.

### Example 2

### Titration Algorithm

Within two weeks before the first dose of Compound 1, serum 25(OH) vitamin D (also known as calcifediol, calcidiol, 25-hydroxycholecalciferol, 25-hydroxyvitamin D₃) should be confirmed to be within the normal range.

At the time of initiation of treatment with Compound 1, serum calcium should be confirmed to be ≥7.8 mg/dL.

If the patient is taking active vitamin D at the time treatment with Compound 1 is initiated and:
if serum calcium is ≥8.3 mg/dL, active vitamin D (calcitriol) is discontinued on the same day as the first dose of Compound 1 is administered and the same dose of calcium supplements is maintained;
if serum calcium is <8.3 mg/dL, the dose of active vitamin D is reduced by ≥50% on the same day as the first dose of Compound 1 is administered and the same dose of calcium supplements is maintained.

If the patient is not taking active vitamin D at the time treatment with Compound 1 is initiated, calcium supplements are decreased by at least 1500 mg on the same day as the first dose of Compound 1 is administered. If calcium supplement doses of 1500 mg calcium/day are taken, calcium supplement doses are discontinued entirely.

If calcium supplements are indicated to meet dietary requirements, a continuation of dietary calcium supplements at doses of <600 mg/day may be considered instead of discontinuing entirely.

The recommended starting dose is 18 μg PTH(1-34) administered in the form of Compound 1 with dose adjustments in 3 μg PTH(1-34) increments thereafter. The dosage range of Compound 1 is 6 to 60 μg PTH(1-34)/day, which doses are provided in prefilled pens of 168 μg PTH(1-34)/0.56 mL (delivering doses of 6, 9 or 12 μg PTH(1-34)); 294 μg PTH(1-34)/0.98 mL (delivering doses of 15, 18, or 21 μg PTH(1-34)); and 420 μg PTH(1-34)/1.4 ml (delivering doses of 24, 27, or 30 μg PTH(1-34)). Each ml of Compound 1 formulation contains 3456 μg of Compound 1, corresponding to 300 μg of PTH(1-34) as active ingredient, and the following inactive ingredients: 1.18 mg succinic acid, 41.7 mg mannitol, 2.5 mg metacresol, 0.13 mg sodium hydroxide, and water for injection, with a pH of 3.7 to 4.3.

Compound 1 is inspected visually for particulate matter and discoloration prior to administration.

Compound 1 is administered subcutaneously daily to the abdomen or front of the thigh and the injection site is rotated daily. For doses of >30 μg PTH(1-34)/day two sequential injections are required, using different sites for each injection.

Serum calcium is measured within 7 to 14 days of the first dose of Compound 1 and appropriate adjustments in dosing of Compound 1, active vitamin D and calcium supplement are as follows:
if serum calcium is low (<8.3 mg/dL):
   if ≥7 days have passed since treatment with Compound 1 was started or the dose of Compound 1 was changed, the same calcium supplement and active vitamin D doses are continued, and the dose of Compound 1 by 3 μg is increased;
   if fewer than 7 days have passed since treatment with Compound 1 was started or the dose of Compound 1 was changed, calcium supplements and/or active vitamin D are increased toward prior doses based on physician's clinical judgement and the same dose of Compound 1 is continued;
if serum calcium is normal (8.3 to 10.6 mg/dL):
   if ≥7 days have passed since treatment with Compound 1 was started or since the dose of Compound 1 was changed and the patient is still taking active vitamin D, active vitamin D is discontinued, and the dose of Compound 1 is increased by 3 μg;
   if ≥7 days have passed since treatment with Compound 1 was started or since the dose of Compound 1 was changed and the patient is no longer taking active vitamin D, but is taking calcium supplements, wherein the calcium supplement is ≥1500 mg/day, the calcium supplement is decreased by ≥1500 mg and the dose of Compound 1 is increased by 3 μg;
   if ≥7 days have passed since treatment with Compound 1 was started or since the dose of Compound 1 was changed and the patient is no longer taking active vitamin D, but is taking calcium supplements, wherein the calcium supplement is less than 1500 mg/day, calcium supplements are discontinued, and the dose of Compound 1 is increased by 3 μg;
   if ≥7 days have passed since treatment with Compound 1 was started or since the dose of Compound 1 was changed and the patient is no longer taking active vitamin D and is no longer taking calcium supplements, the same dose of Compound 1 is continued;
   if fewer than 7 days have passed since treatment with Compound 1 was started or since the dose of Compound 1 was changed, the same dose of Compound 1, of the calcium supplement and active vitamin D is continued;
if serum calcium is high (10.7 to 11.9 mg/dL):
   if the patient is still taking active vitamin D, active vitamin D is discontinued and the same doses of Compound 1 and calcium supplement are continued;
   if the patient is not taking active vitamin D but takes calcium supplements at a dose of ≥1500 mg/day, calcium supplements are decreased by ≥1500 mg and the same dose of Compound 1 is continued;
   if the patient is not taking active vitamin D but takes calcium supplements, which calcium supplements are less than 1500 mg per day, calcium supplements are discontinued, and the same dose of Compound 1 is continued;
   if the patient is not taking active vitamin D and does not take calcium supplements, the dose of Compound 1 is decreased by 3 μg;
and if serum calcium is very high (>12 mg/dL), withhold Compound 1 for 2 to 3 days, recheck serum calcium and:
   if subsequent serum calcium is <12 mg/dL, resume titration of Compound 1, active vitamin D and calcium supplements as described above using the most recent serum calcium value obtained; and
   if serum calcium remains ≥12 mg/dL, withhold Compound 1 for an additional 2 to 3 days, recheck serum calcium and proceed as described above.

Doses of >30 μg/day are administered as two single doses injected sequentially at different injection sites using two administrations as shown in Table 1.

TABLE 1
Scheme for doses of >30 μg/day

| Dose | Dosing Scheme |
|---|---|
| 33 μg PTH(1-34)/day | 15 μg PTH(1-34)/day + 18 μg PTH(1-34)/day |
| 36 μg PTH(1-34)/day | 18 μg PTH(1-34)/day + 18 μg PTH(1-34)/day |
| 39 μg PTH(1-34)/day | 18 μg PTH(1-34)/day + 21 μg PTH(1-34)/day |
| 42 μg PTH(1-34)/day | 21 μg PTH(1-34)/day + 21 μg PTH(1-34)/day |
| 45 μg PTH(1-34)/day | 21 μg PTH(1-34)/day + 24 μg PTH(1-34)/day |
| 48 μg PTH(1-34)/day | 24 μg PTH(1-34)/day + 24 μg PTH(1-34)/day |
| 51 μg PTH(1-34)/day | 24 μg PTH(1-34)/day + 27 μg PTH(1-34)/day |
| 54 μg PTH(1-34)/day | 27 μg PTH(1-34)/day + 27 μg PTH(1-34)/day |
| 57 μg PTH(1-34)/day | 27 μg PTH(1-34)/day + 30 μg PTH(1-34)/day |
| 60 μg PTH(1-34)/day | 30 μg PTH(1-34)/day + 30 μg PTH(1-34)/day |

Dose adjustments of Compound 1, active vitamin D and calcium supplements should be made on the same day. After any dose change in Compound 1, active vitamin D or calcium supplements, measure serum calcium within 7 to 14 days and monitor for clinical symptoms of hypocalcemia or hypercalcemia and adjust doses of Compound 1, active vitamin D and/or calcium supplements as described above.

The dose of Compound 1 may be increased as described above in increments of 3 μg if at least 7 days have elapsed since a prior dose change of Compound 1. Do not increase the dose of Compound 1 more often than every 7 days. The dose of Compound 1 may be reduced no more often than every 3 days in 3 μg increments in response to hypercalcemia as described above.

The maintenance dose should be the dose of Compound 1 that achieves serum calcium within the normal range, without the need for active vitamin D or therapeutic doses of calcium.

Optionally, calcium supplementation sufficient to meet dietary requirements may be continued. Serum calcium may be measured per standard of care once a maintenance dose is achieved.

If a dose is missed by less than 12 hours, it may be taken as soon as possible. If a dose is missed by more than 12 hours, it may be skipped, and the next dose is then taken as scheduled.

Interruption of daily administration should be avoided to minimize serum PTH fluctuations. Interruption or discontinuation of Compound 1 may result in hypocalcemia. In patients interrupting or discontinuing Compound 1 for 3 or more consecutive doses, it is recommended to monitor for signs and symptoms of hypocalcemia and to consider measuring serum calcium. If indicated, treatment with calcium supplements and active vitamin D is resumed. It is recommended that administration of Compound 1 is resumed at the prescribed dose as soon as possible after an interruption. When resuming Compound 1 after an interruption, serum calcium should be measured and doses of Compound 1, active vitamin D and calcium supplements adjusted as described above.

If required for using dose prediction formulas of Example 7 and 8 below, it may be required to report the dose in molar equivalents. It is standard in the art to convert the mass dose to the molar dose. As an example, for PTH(1-34) with a molecular mass of 4117.8 Daltons, the molar dose is 4.37 nmol.

### Example 3

### Titration Off Active Vitamin D and Therapeutic Calcium Supplementation

Treatment with Compound 1 was carried out in accordance with Example 1. Data (representing mean values for the treatment group) at various timepoints throughout the treatment, are provided in Table 2 below.

TABLE 2
Mean values of serum calcium, calcium and active
vitamin D doses at various time points

| | | Calcium | Active | | Compound |
|---|---|---|---|---|---|
| | Serum | supplement | Vitamin D | Compound | 1 Molar |
| Time | Calcium | dose | dose | 1 dose | dose |
| (Weeks) | (mg/dL) | (mg) | (μg) | (μg/day) | (nmol/day) |
| 0 | 8.8 | 1748.03 | 0.992 | 18 | 4.37 |
| 2 | 9.8 | 1524.1 | 0.098 | 20.3 | 4.93 |
| 4 | 9.6 | 548.77 | 0.008 | 21.4 | 5.20 |
| 6 | 9.7 | 229.1 | 0.004 | 21.2 | 5.15 |
| 8 | 9.5 | 105.74 | 0 | 21.2 | 5.15 |
| 10 | 9.5 | 114.17 | 0 | 20.2 | 4.91 |
| 12 | 9.5 | 102.46 | 0 | 20.8 | 5.05 |
| 16 | 9.3 | 182.38 | 0 | 21.3 | 5.17 |
| 20 | 9.3 | 300.83 | 0 | 21.2 | 5.15 |
| 26 | 8.9 | 274.17 | 0 | 21.4 | 5.20 |

Treatment with Compound 1 enabled rapid and sustained elimination of therapeutic levels of elemental calcium and complete discontinuation of active vitamin D within 8 weeks. At week 26, the mean Compound 1 dose in participants allocated to active therapy was 21.4 μg/day (median 21 μg/day). The range of Compound 1 doses was 9-39 μg/day, demonstrating the wide individual PTH requirement.

When adequately titrated, participants treated with Compound 1 maintained mean serum calcium (sCa) levels in the normal range at all study visits. Five participants (8.2%) in the Compound 1 group reported SAEs, one of which was considered related to treatment. This participant experienced hypercalcemia in the setting of an inadvertent deviation from the titration algorithm and required hospitalization and per-protocol interruption of treatment.

Treatment with Compound 1 could be continued as serum calcium normalized.

In this randomized trial of women and men with chronic hypoparathyroidism, PTH replacement therapy with Compound 1 robustly improved and maintained mean serum calcium levels in the normal range at all study visits, while allowing independence from conventional therapy.

In conclusion, the ability to titrate the dose of Compound 1 offers an individualized approach to safely determine the individual daily PTH requirement in the absence of conventional therapy.

### Example 4

Identification of patient population, titrated off standard of care and having a stable PTH requirement, without further dose titration

Human participants with hypoparathyroidism were randomly assigned to one of four groups: three groups received fixed doses of Compound 1 and one group received placebo. Compound 1 or placebo were administered as a subcutaneous injection using a pre-filled injection pen.

Neither trial participants nor their doctors knew who were assigned to each group. After the four weeks, participants were eligible to continue in the trial as part of a long-term extension study. During the extension, all participants received Compound 1, with the dose adjusted to their individual needs.

The double-blind, placebo-controlled, parallel group treatment period of this trial was designed to enroll approximately 55 male and female adults with either postsurgical HP or autoimmune, genetic, or idiopathic HP for at least 26 weeks, from up to approximately 40 sites worldwide.

The ClinicalTrials.gov Identifier is NCT04009291.

Subjects were randomized into 4 treatment groups (1:1:1:1):
Compound 1 15 μg/day*
Compound 1 18 μg/day*
Compound 1 21 μg/day*
Placebo for compound 1 (excipients solution)

(*Dose of compound 1 refers to dose of PTH(1-34) administered measured in PTH equivalents) To maintain blinding, the placebo group were sub-randomized into 3 groups (1:1:1) to mimic doses of 15, 18, and 21 g/day. Subjects remained on the same dose of study drug throughout the 4-week Blinded Treatment Period. Following successful completion of the Blinded Treatment Period, subjects entered open-label Extension Period at which time all subjects received compound 1.

During the extension period it was surprisingly discovered that a subset of patients was stable in their PTH requirement and did not require dose titration (see Table 3 below). These patients had been successfully titrated off conventional therapy and their individual PTH requirement had been identified. These patients would be the most suitable candidates for switching to weekly PTH.

TABLE 3
Identification of a patient population, titrated off standard of care
and having a stable PTH requirement, without further dose titration
Dose Change from Week 58 to Week 84

| | Proportion (%, n/N) |
|---|---|
| Dose Decrease | 0 |
| No Dose Change | 50 (29/58) |
| 1 Dose Increase | 31.03 (18/58) |
| ≥1 Dose Increase | 18.97 (11/58) |

### Example 5

### PK and PD Study of Compounds 2 and 3 in Cynomolgus Monkeys

Compound 2 and Compound 3 are investigational long-acting Parathyroid Hormone (PTH) prodrugs for potential treatment of certain parathyroid related disorders. Both Compound 2 and Compound 3 are inactive prodrugs comprised of PTH(1-34) transiently conjugated via linkers to carriers (in both cases a 40 kDa mPEG moiety). At physiologic pH and temperature, active PTH is released from the prodrug.

The objectives of this study were to determine and compare the Pharmacokinetic (PK) and Pharmacodynamic (PD) effects of Compound 2 and Compound 3 when administered by the subcutaneous (SC) route once weekly for 7 weeks to Cynomolgus monkeys.

The animals were fed a diet of Expanded complete commercial primate diet (special diet services: OWM [E]banana SQC short) (0.55% Calcium) and had free access to municipal tap water, softened and filtered (0.2 m). The animals were grouped according to Table 4 below.

TABLE 4

| | | | Dose Level | |
|---|---|---|---|---|
| | | Dose Level (μg | (nmol PTH/kg | |
| Group | | PTH/kg animal | animal body | Number of |
| number | Test Item | body mass* | mass | animals |
| 1 | Compound 2 | 15 | 3.64 | 3 |
| 2 | Compound 3 | 15 | 3.64 | 3 |

*Dose is reported as PTH(1-34) equivalents.

Once weekly, SC dosing was carried out for a duration of 7 administrations (at Days 1, 8, 15, 22, 29, 36, 43).

The following parameters and end points were evaluated in this study: Clinical signs, injection site observations, body weight, body weight gain and clinical pathology parameters (hematology, coagulation, clinical chemistry and urinalysis).

Blood samples for PK and PD evaluations were collected on the days of test item administration at different time points pre- and postdosing.

SC administration of Compound 2 or Compound 3 to female Cynomolgus monkeys once weekly for 7 weeks was well tolerated at levels of 15 g PTH(1-34)/kg/dose. No test item-related changes were identified at clinical and injection site observations or body weight/body weight gain measurements.

PK Methods Determination of plasma concentrations of PTH(1-34) was carried out at PRA Health Sciences in Assen, Netherlands, using high performance liquid chromatography with tandem mass spectrometry (LC-MS/MS) using qualified methods.

The concentrations of PTH(1-34) were determined in acidified plasma, because of instability of the prodrug in neat plasma. The study samples were prepared by diluting whole blood exactly 1.2 times with citrate buffer (pH 4), implying a dilution factor in plasma of approximately 1.4 times, with the assumption that approximately one half of the volume of whole blood is plasma.

PK evaluation was performed by noncompartmental analysis using the Phoenix® WinNonlin® Software (Version 8.0).

### Results

Expected pharmacological transient increases in serum Calcium (sCa) and phosphate concentrations were observed at a similar magnitude in both groups.

Systemic exposure of PTH(1-34), was evident up to 168 h after administration of both Compound 2 and Compound 3. This was accompanied by the suppression of the PD marker, PTH(1-84), for 168 h and an increase of sCa, above normal range, for up to 72 h postdosing.

Under the conditions of this study, subcutaneous administration of Compound 2 or Compound 3 to female Cynomolgus monkeys once weekly for 7 weeks (a total of 7 administrations) was well tolerated at levels of 15 g PTH(1-34)/kg/dose.

Overall, the PK and PD responses observed after administration of both candidates were considered comparable (see Table 5 below).

TABLE 5
PK and PD responses of compound 2 and compound 3

| | | | Cₘₐₓ | AUC_{0-168 h} | T_{1/2} | CL |
|---|---|---|---|---|---|---|
| Group | Week | Tₘₐₓ | (ng/mL) | (h*ng/mL) | (hour) | (L/h) |
| Compound 2 | 1 | 24 | 0.0372 | 3.58 | 96.2 | — |
| | 6 | 24 | 0.0434 | 4.46 | 90.0 | 8.5 |
| Compound 3 | 1 | 24 | 0.0511 | 4.17 | 64.8 | — |
| | 6 | 12 | 0.0522 | 5.18 | 83 | 6.7 |

After the administration of Compound 2 or Compound 3, sCa concentrations largely remained above the historical reference range throughout the 6 weeks study period. After each individual dosing, an increase in sCa concentrations was observed for at least 72 hours and returned to baseline or just below baseline 168 hours after dosing.

In conclusion, subcutaneous administration of Compound 2 or Compound 3 to female Cynomolgus monkeys once weekly for 7 weeks at levels of 15 g PTH(1-34)/kg/dose (a total of 7 administrations) was well tolerated.

Transient increases in sCa and sP concentrations at a similar magnitude in both groups were observed after dosing and were considered expected pharmacological effects of the test items.

The PK and PD responses observed after administration of Compound 2 and Compound 3 were considered comparable and supportive of weekly PTH supplementation in humans.

### Example 6

### Allometric Scaling of Clearance and Half-Life for Compounds 2 and 3

Prediction of clearance and half-life of Compounds 2 and 3 in humans was based on allometric scaling of the PK parameters obtained in monkeys (see Example 5). The PK parameters clearance (CL) and half-life (t½) were scaled as a function of body mass using formulas listed below (see: Antibodies 2022, 11(2), 42; https://www.mdpi.com/2073-4468/11/2/42). Body masses were 2.33-2.53 for monkey and 70 kg for human.

$$Predicted CL in human = CL in animal * {( \frac{Human body weight}{Animal body weight} )}^{0.85} \qquad 1$$

$$Predicted {t}_{1 / 2} in human = {t}_{1 / 2} in animal * {( \frac{Human body weight}{Animal body weight} )}^{0.15} \qquad 2$$

Table 6 below sets out CL and t½ values obtained for the monkey (Example 5) and the human (based on application of the above formulas).

TABLE 6

| | | Clearance | Half-life | |
|---|---|---|---|---|
| Species | Compound | (L/h) | (h) | |
| Monkey | 2 | 8.5 | 90 | Calculated in |
| | | | | Example 5 |
| Monkey | 3 | 6.7 | 83 | Calculated in |
| | | | | Example 5 |
| Human | 2 | 143 | 148 | Scaled based on |
| | | | | Formula 1 and 2 |
| Human | 3 | 122 | 138 | Scaled based on |
| | | | | Formula 1 and 2 |

### Example 7

### Determination of Suitable Weekly Dosing of PTH Compounds 2 and 3 in Humans

Once it has been established what the individual PTH requirement is for Compound 1, and it has been determined what average steady state blood concentration of PTH(1-34) results from this PTH requirement, it is then possible to predict an equivalent weekly dose of PTH compounds 2 or 3 using the following formula:

$${C}_{avg , ss} = \frac{F * Dose}{CL * dosing interval} \qquad 3$$

wherein:
C_{avg,ss} is the average steady state blood concentration
Dose is the estimate weekly dose
F is bioavailability set to 1 for Compounds 2 and 3;
CL is clearance (predicted in Table 6 for Compounds 2 and 3); and
dosing interval is 168 hours (weekly dosing)

For example, consider a patient that has been treated with Compound 1 and an individualized PTH dose of Compound 1 has been determined to be 23.6 μg/day, and that this dose resulted in a blood concentration of PTH(1-34) of 7.4 μg/mL. Using the determined blood concentration of PTH(1-34), then a suitable weekly starting dose of a PTH compounds 2 or 3 can be predicted using the formula (3) above, by rearranging.

$$Dose = \frac{C a v e , s s * CL * Dosing interval}{F} \qquad 4$$

Based on the above formula, the weekly dose resulting in a comparable blood concentration of PTH(1-34) following treatment with Compound 2 having a CL of 143 L/h, will thus be 178 μg.

The weekly dose resulting in a comparable blood concentration of PTH(1-34) following treatment with Compound 3 having a CL of 122 L/h will thus be 151 μg.

### Example 8

As it is routine to calculate CL of compounds in clinical development, Formula 3 can be modified to enable prediction of the dose of the weekly PTH compound when CL has been determined, provided the CL and dose of the first compound is known and that the relative potency of the first and the second compound is the same. This is the case for Compounds 1, 2 and 3 where the released active moiety in all cases is unmodified PTH 1-34. Thus:

$$\frac{F * Dose1}{CL 1 * dosing interval1} = \frac{F * Dose2}{CL 1 * dosing interval2}$$

Which can be reorganized to:

$$Dose 2 = \frac{Dose 1 * Dosing interval 2 * CL 2}{CL 1 * dosing interval 1} \qquad 5$$

Using the formula (5) above it is possible to predict the equivalent weekly dose of PTH Compound 2 or 3 as outlined in Table 7 below.

TABLE 7
Prediction of equivalent weekly dose

| | Molar | | Molar | | Molar |
|---|---|---|---|---|---|
| Dose of | Dose of | Dose of | dose of | Dose | dose of |
| Compound 1 | Compound | Compound 2 | Compound | Compound 3 | Compound |
| μg PTH | 1 nmol | μg PTH | 2 nmol | μg PTH | 3 nmol |
| (daily) | PTH (daily) | (weekly) | PTH (weekly) | (weekly) | PTH (weekly) |
| 18 | 4.37 | 135 | 32.78 | 116 | 28.17 |
| 21 | 5.10 | 158 | 38.37 | 135 | 32.78 |
| 24 | 5.83 | 180 | 43.71 | 154 | 37.40 |

If the first and second PTH compound differ in their active PTH, prediction of the equivalent weekly dose can be done based on suppression of endogenous PTH 1-84 in healthy volunteers.

The conversion factor between the dose of the first and second PTH compounds can be determined as the ratio between the dose of the second and first PTH compound required to fully suppress endogenous PTH 1-84 secretion in healthy volunteers following repeated administration at the respective intended dosing frequency for at least until steady state is reached.

For example, for Compound 1, 16 μg/day fully suppresses endogenous PTH 1-84 in healthy volunteers (Journal of Bone and Mineral Research, Vol. 35, No. 8, August 2020, pp 1430-1440) with no further suppression at higher doses.

To determine the equivalent dose of a second PTH compound, the dose required to suppress PTH 1-84 can be determined in a similar fashion, allowing for a relative potency comparison. If for sake of argument the second PTH compound requires a weekly dose of 300 μg to suppress endogenous PTH 1-84 in healthy volunteers, then the conversion factor would be 300 μg weekly/(7*16 μg)=2.7. Thus, if the individual PTH requirement for Compound 1 has been determined to 21 μg/day, or 147 μg per week, then the weekly dose of the second PTH compound would be 397 μg.

Similarly, for daily PTH Compound 5, 60 μg is able to suppress endogenous PTH 1-84 in healthy volunteers (Journal of Bone and Mineral Research, Vol. 35, No. 8, August 2020, pp 1430-1440) with no further suppression at higher doses.

If the dose of a second PTH Compound required to suppress endogenous PTH 1-84 in healthy volunteers, is, for example, determined to be 300 μg administered weekly, the conversion factor would be 300 μg weekly/(7*60 μg)=0.7. Thus, if the individual PTH requirement for Compound 5 has been determined to 30 μg/day, or 210 μg per week, then the weekly dose of the second PTH compound would be 147 μg.

If the first PTH compound is administered as multiple daily injections, it is understood that it is the daily dose that is multiplied by 7 to obtain the weekly dose.

### Example 9

### Transitioning Patients from Daily to Weekly Dosed PTH Compounds

Patients who no longer require PTH dose titration to achieve stable serum calcium levels, within the normal physiological range, in the absence of active vitamin D and calcium supplementation (above 600 mg/day), are ideally suited for switching to a weekly PTH treatment.

For example, a patient is treated with Compound 1 in accordance with Example 1 until calcium supplement (greater than 600 mg) and active vitamin D have been successfully discontinued, and the patient's serum calcium is maintained within the normal physiological range.

Once discontinuation of vitamin D/calcium has been achieved and serum calcium levels are within the normal physiological range, the patient is switched to a weekly dosing of PTH, using one or a combination of the following PTH compounds suitable for weekly dosing:

Compound 2, administered at a weekly dose which is calculated based on the PTH requirement determined during the daily dosing period. The starting weekly dose can be calculated as described in Example 8 above.

Compound 3, administered at a weekly dose which is calculated based on the PTH requirement determined during the daily dosing period. The starting weekly dose can be calculated as described in Example 8 above.

Compound 4, administered at a weekly dose which is calculated based on the PTH requirement determined during the daily dosing period. The starting weekly dose can be calculated as described in Example 8 above (i.e., by experimentally determining and applying the conversion factor between Compound 1 and Compound 4).

1. A method of treating chronic hypoparathyroidism, the method comprising:
(a) administering to a patient having chronic hypoparathyroidism a first PTH compound on multiple occasions with a first average interval between administrations, adjusting dose if needed, to determine for the patient a dose at which the patient's serum calcium level remains within normal range in the absence of active vitamin D and calcium supplement;
(b) using the dose determined in step (a) to determine an initial dose for a second PTH compound to be administered on multiple occasions to the patient, with a second average interval, longer than the first average interval; and
(c) administering the second PTH compound on multiple occasions with the second average interval between administrations to the patient starting at the initial dose determined in step (b) with adjustment of the dose, if needed, until the patient's serum calcium remains within normal range in the absence of active vitamin D and calcium supplement.

2. The method of claim 1, wherein the first average interval between administrations 8 hours.

3. The method of claim 1, wherein the first average interval between administrations 12 hours.

4. The method of claim 1, wherein the first average interval between administrations is one day.

5. The method of claim 1, wherein the second average interval between administrations is one week.

6. The method of claim 1, wherein the patient is a mammalian patient.

7. The method of claim 1, wherein the patient is a human patient.

8. The method of claim 1, wherein the patient is an adult patient.

9. The method of claim 1, wherein the patient is a pediatric patient.

10. The method of claim 1, wherein the first PTH compound is administered to a patient via topical, enteral or parenteral administration or by methods of external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection and infusion, direct delivery to the brain via implanted device allowing delivery of the invention to brain tissue or brain fluids, direct intracerebroventricular injection or infusion, injection or infusion into brain or brain associated regions, injection into the subchoroidal space, retro-orbital injection or ocular instillation.

11. The method of claim 1, wherein the first PTH compound is administered by subcutaneous administration, such as by subcutaneous injection.

12. The method of claim 1, wherein the second PTH compound is administered to a patient via topical, enteral or parenteral administration or by methods of external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection and infusion, direct delivery to the brain via implanted device allowing delivery of the invention to brain tissue or brain fluids, direct intracerebroventricular injection or infusion, injection or infusion into brain or brain associated regions, injection into the subchoroidal space, retro-orbital injection or ocular instillation.

13. The method of claim 1, wherein the second PTH compound is administered by subcutaneous administration, such as by subcutaneous injection.

14. The method of claim 1, wherein the first and/or second PTH compound is/are independently a compound of formula (Ia) or (Ib) or a pharmaceutically acceptable salt thereof 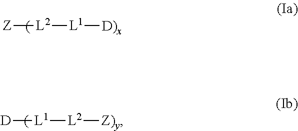
wherein
each -D is independently a PTH moiety;
each -L¹- is independently a linker moiety covalently and reversibly connected to -D;
each -L²- is independently a single chemical bond or a spacer moiety;
each —Z is independently a carrier moiety, such as a fatty acid derivative or a polymer;
x is an integer selected from the group consisting of 1, 2, 3, 4, 5, 6, 7, 8, 9, 10, 11, 12, 13, 14, 15, 16, 17, 18, 19, 20, 21, 22, 23, 24 and 25; and
y is an integer selected from the group consisting of 2, 3, 4 and 5.

15. The method of claim 1, wherein the first PTH compound is a compound of formula (I) 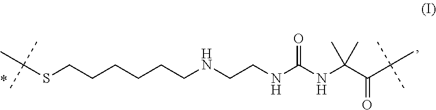
wherein
the unmarked dashed line indicates the attachment to the nitrogen of the N-terminal amine group of a PTH moiety of SEQ ID NO:51; and
the dashed line marked with the asterisk indicates attachment to a moiety 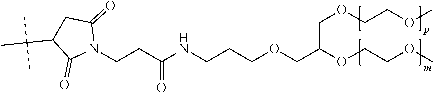
wherein
m and p are independently an integer ranging from approx. 400 to 500.

16. The method of claim 1, wherein the first PTH compound is PTH 1-34.

17. The method of claim 1, wherein the first PTH compound is PTH 1-84.

18. The method of claim 1, wherein the first PTH compound has the sequence of SEQ ID NO: 122.

19. The method of claim 1, wherein the second PTH compound is a compound of formula (II-b) 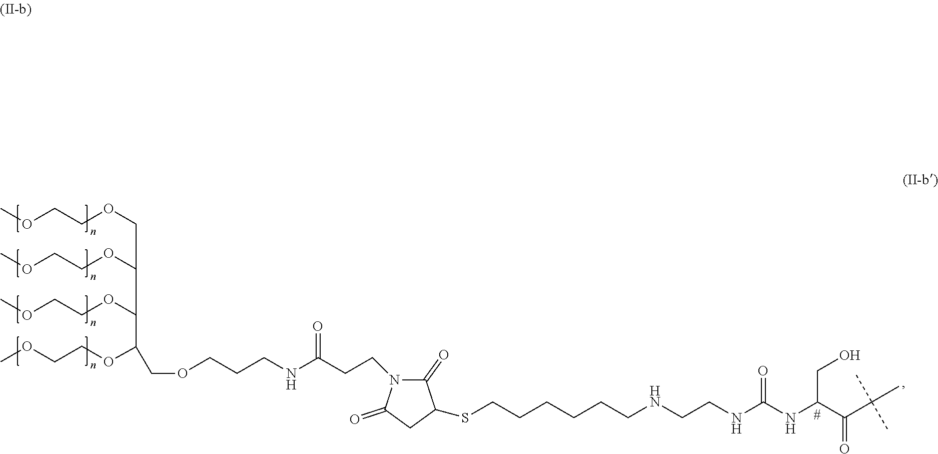
wherein
the dashed line indicates attachment to the nitrogen of the N-terminal amine group of a PTH moiety having the sequence of SEQ ID NO:51; and
each n is independently an integer ranging from approx. 200 to 250.

20. The method of claim 1, wherein the PTH compound administered weekly is a compound of formula (II-b′) 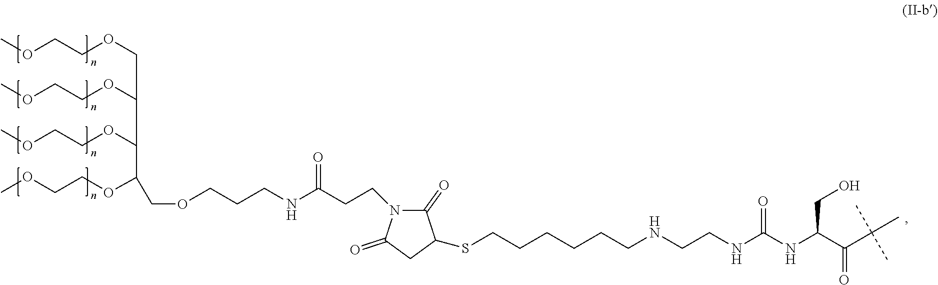
wherein
the dashed line indicates attachment to the nitrogen of the N-terminal amine group of a PTH moiety having the sequence of SEQ ID NO:51; and
each n is independently an integer ranging from approx. 200 to 250.

21. The method of claim 19, wherein each n is independently an integer ranging from approx. 210 to 240.

22. The method of claim 19, wherein each n is independently an integer ranging from 210 to 240.

23. The method of claim 1, wherein the second PTH compound is a compound of formula (II-i)
(II-i)
k(γE-(miniPEG)₂-γE-COC₁₆H₃₂CO₂H)(N-Me)GSVSEIQL
MHNLGKHLNSMERVEWLRKKLQDVHK(γE-(miniPEG)₂-γE-
COC₁₆H₃₂CO₂H)-OH,
wherein
k is d-Lys;
γE is the 1-isomer of gamma, glutamic acid;
miniPEG is COCH₂OCH₂CH₂OCH₂CH₂NH;
COC₁₆H₃₂CO₂H is C18 diacid;
(N-Me)G is sarcosine;
K is 1-isomer of lysine; and
—OH designates the C-terminal amino acid has a terminal carboxylic acid.

24. The method of claim 1, wherein the second PTH compound is a compound of formula(II-i′)
(II-i′)
k(γE-(miniPEG)₂-γE-COC₁₆H₃₂CO₂H)(N-Me)GSVSEIQL
MHNLGKHLNSMERVEWLRKKLQDVHK(γE-(miniPEG)₂-γE-
COC₁₆H₃₂CO₂H)-OH,
wherein
k is d-Lys;
γE is the 1-isomer of gamma, glutamic acid;
(miniPEG)₂ is COCH₂OCH₂CH₂OCH₂CH₂NH;
COC₁₆H₃₂CO₂H is C18 diacid;
(N-Me)G is sarcosine;
K is 1-isomer of lysine; and
—OH designates the C-terminal amino acid has a terminal carboxylic acid.

25. The method of claim 1, wherein the active PTH of the first and second PTH compound are the same and the initial dose of the second PTH compound is D₂±25% and wherein D₂ is calculated using the equation
$${D}_{2} = {D}_{1} \times \frac{{T}_{2}}{{T}_{1}}$$
wherein
D₁ is the dose of the first PTH compound at which the patient's serum calcium level remains within normal range in the absence of active vitamin D and calcium supplement;
T₁ is the first interval between administrations; and
T₂ is the second interval between administrations.

26. The method of claim 1 to 24, wherein the active PTH of the first and the second PTH compound are different and the initial dose of the second PTH compound is calculated by first calculating a correction factor CF using the following equation
$$CF = \frac{{D}_{supp 2}}{{D}_{supp 1}} \times \frac{{T}_{supp 1}}{{T}_{supp 2}}$$
wherein
Dₛᵤₚₚ₁ is the dose of the first PTH compound that fully suppresses secretion of endogenous PTH 1-84 in healthy volunteers;
Dₛᵤₚₚ₂ is the dose of the second PTH compound that fully suppresses secretion of endogenous PTH 1-84 in healthy volunteers;
Tₛᵤₚₚ₁ is the length of the first interval between administrations of the first PTH compound to the healthy volunteers; and
Tₛᵤₚₚ₂ is the length of the second interval between administrations of the second PTH compound to the healthy volunteers;
followed by calculating D2 using the following equation
$${D}_{2} = {D}_{1} \times CF \times \frac{{T}_{2}}{{T}_{1}}$$
wherein
D₁ is the dose of the first PTH compound at which the patient's serum calcium level remains within normal range in the absence of active vitamin D and calcium supplement;
T₁ is the first interval between administrations;
T₂ is the second interval between administrations;
and wherein the initial dose of the second PTH compound is D₂±25%.

27. The method of claim 1, wherein the time between the last dose of the first PTH compound and the first dose of the second PTH compound corresponds to the average interval between two administrations of the first PTH compound.

28. The method of claim 1, wherein the first PTH compound is the only drug comprising PTH or a PTH moiety administered to the patient in the first treatment period.

29. The method of claim 1, wherein the second PTH compound is the only drug comprising PTH or a PTH moiety administered to the patient during treatment with the second PTH compound.

30. The method of claim 1, wherein the treatment period with the first PTH compound lasts from one week to 20 years.

31. The method of claim 1, wherein the treatment period with the second PTH compound lasts for as long as the patient benefits from administration of the second PTH compound.

32. The method of claim 1, wherein the chronic hypoparathyroidism is due to surgery, such as thyroid or parathyroid gland surgery, a genetic cause, immune system-related damage of the parathyroid glands or is idiopathic.

33. The method of claim 1, wherein the chronic hypoparathyroidism is due to surgery.

34. The method of claim 1, wherein the chronic hypoparathyroidism is due to a genetic cause.

35. The method of claim 1, wherein the chronic hypoparathyroidism is due to immune system-related damage of the parathyroid glands.

36. The method of claim 1, wherein the chronic hypoparathyroidism is idiopathic.

37-59. (canceled)


SEQUENCE LISTING (134 sequences)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 2 - protein, 83 aa, synthetic construct
  REGION 1..83: note = Human PTH 1-83
SVSEIQLMHNLGKHLNSMERVEWLRKKLQDVHNFVALGAPLAPRDAGSQRPRKKEDNVLVESHEKSLGEADKADVNVLTKAKS

SEQ ID NO: 3 - protein, 82 aa, synthetic construct
  REGION 1..82: note = human PTH 1-82
SVSEIQLMHNLGKHLNSMERVEWLRKKLQDVHNFVALGAPLAPRDAGSQRPRKKEDNVLVESHEKSLGEADKADVNVLTKAK

SEQ ID NO: 4 - protein, 81 aa, synthetic construct
  REGION 1..81: note = human PTH 1-81
SVSEIQLMHNLGKHLNSMERVEWLRKKLQDVHNFVALGAPLAPRDAGSQRPRKKEDNVLVESHEKSLGEADKADVNVLTKA

SEQ ID NO: 5 - protein, 80 aa, synthetic construct
  REGION 1..80: note = human PTH 1-80
SVSEIQLMHNLGKHLNSMERVEWLRKKLQDVHNFVALGAPLAPRDAGSQRPRKKEDNVLVESHEKSLGEADKADVNVLTK

SEQ ID NO: 6 - protein, 79 aa, synthetic construct
  REGION 1..79: note = human PTH 1-79
SVSEIQLMHNLGKHLNSMERVEWLRKKLQDVHNFVALGAPLAPRDAGSQRPRKKEDNVLVESHEKSLGEADKADVNVLT

SEQ ID NO: 7 - protein, 78 aa, synthetic construct
  REGION 1..78: note = human PTH 1-78
SVSEIQLMHNLGKHLNSMERVEWLRKKLQDVHNFVALGAPLAPRDAGSQRPRKKEDNVLVESHEKSLGEADKADVNVL

SEQ ID NO: 8 - protein, 77 aa, synthetic construct
  REGION 1..77: note = human PTH 1-77
SVSEIQLMHNLGKHLNSMERVEWLRKKLQDVHNFVALGAPLAPRDAGSQRPRKKEDNVLVESHEKSLGEADKADVNV

SEQ ID NO: 9 - protein, 76 aa, synthetic construct
  REGION 1..76: note = human PTH 1-76
SVSEIQLMHNLGKHLNSMERVEWLRKKLQDVHNFVALGAPLAPRDAGSQRPRKKEDNVLVESHEKSLGEADKADVN

SEQ ID NO: 10 - protein, 75 aa, synthetic construct
  REGION 1..75: note = human PTH 1-75
SVSEIQLMHNLGKHLNSMERVEWLRKKLQDVHNFVALGAPLAPRDAGSQRPRKKEDNVLVESHEKSLGEADKADV

SEQ ID NO: 11 - protein, 74 aa, synthetic construct
  REGION 1..74: note = human PTH 1-74
SVSEIQLMHNLGKHLNSMERVEWLRKKLQDVHNFVALGAPLAPRDAGSQRPRKKEDNVLVESHEKSLGEADKAD

SEQ ID NO: 12 - protein, 73 aa, synthetic construct
  REGION 1..73: note = human PTH 1-73
SVSEIQLMHNLGKHLNSMERVEWLRKKLQDVHNFVALGAPLAPRDAGSQRPRKKEDNVLVESHEKSLGEADKA

SEQ ID NO: 13 - protein, 72 aa, synthetic construct
  REGION 1..72: note = human PTH 1-72
SVSEIQLMHNLGKHLNSMERVEWLRKKLQDVHNFVALGAPLAPRDAGSQRPRKKEDNVLVESHEKSLGEADK

SEQ ID NO: 14 - protein, 71 aa, synthetic construct
  REGION 1..71: note = human PTH 1-71
SVSEIQLMHNLGKHLNSMERVEWLRKKLQDVHNFVALGAPLAPRDAGSQRPRKKEDNVLVESHEKSLGEAD

SEQ ID NO: 15 - protein, 70 aa, synthetic construct
  REGION 1..70: note = human PTH 1-70
SVSEIQLMHNLGKHLNSMERVEWLRKKLQDVHNFVALGAPLAPRDAGSQRPRKKEDNVLVESHEKSLGEA

SEQ ID NO: 16 - protein, 69 aa, synthetic construct
  REGION 1..69: note = human PTH 1-69
SVSEIQLMHNLGKHLNSMERVEWLRKKLQDVHNFVALGAPLAPRDAGSQRPRKKEDNVLVESHEKSLGE

SEQ ID NO: 17 - protein, 68 aa, synthetic construct
  REGION 1..68: note = human PTH 1-68
SVSEIQLMHNLGKHLNSMERVEWLRKKLQDVHNFVALGAPLAPRDAGSQRPRKKEDNVLVESHEKSLG

SEQ ID NO: 18 - protein, 67 aa, synthetic construct
  REGION 1..67: note = human PTH 1-67
SVSEIQLMHNLGKHLNSMERVEWLRKKLQDVHNFVALGAPLAPRDAGSQRPRKKEDNVLVESHEKSL

SEQ ID NO: 19 - protein, 66 aa, synthetic construct
  REGION 1..66: note = human PTH 1-66
SVSEIQLMHNLGKHLNSMERVEWLRKKLQDVHNFVALGAPLAPRDAGSQRPRKKEDNVLVESHEKS

SEQ ID NO: 20 - protein, 65 aa, synthetic construct
  REGION 1..65: note = human PTH 1-65
SVSEIQLMHNLGKHLNSMERVEWLRKKLQDVHNFVALGAPLAPRDAGSQRPRKKEDNVLVESHEK

SEQ ID NO: 21 - protein, 64 aa, synthetic construct
  REGION 1..64: note = human PTH 1-64
SVSEIQLMHNLGKHLNSMERVEWLRKKLQDVHNFVALGAPLAPRDAGSQRPRKKEDNVLVESHE

SEQ ID NO: 22 - protein, 63 aa, synthetic construct
  REGION 1..63: note = human PTH 1-63
SVSEIQLMHNLGKHLNSMERVEWLRKKLQDVHNFVALGAPLAPRDAGSQRPRKKEDNVLVESH

SEQ ID NO: 23 - protein, 62 aa, synthetic construct
  REGION 1..62: note = human PTH 1-62
SVSEIQLMHNLGKHLNSMERVEWLRKKLQDVHNFVALGAPLAPRDAGSQRPRKKEDNVLVES

SEQ ID NO: 24 - protein, 61 aa, synthetic construct
  REGION 1..61: note = human PTH 1-61
SVSEIQLMHNLGKHLNSMERVEWLRKKLQDVHNFVALGAPLAPRDAGSQRPRKKEDNVLVE

SEQ ID NO: 25 - protein, 60 aa, synthetic construct
  REGION 1..60: note = human PTH 1-60
SVSEIQLMHNLGKHLNSMERVEWLRKKLQDVHNFVALGAPLAPRDAGSQRPRKKEDNVLV

SEQ ID NO: 26 - protein, 59 aa, synthetic construct
  REGION 1..59: note = human PTH 1-59
SVSEIQLMHNLGKHLNSMERVEWLRKKLQDVHNFVALGAPLAPRDAGSQRPRKKEDNVL

SEQ ID NO: 27 - protein, 58 aa, synthetic construct
  REGION 1..58: note = human PTH 1-58
SVSEIQLMHNLGKHLNSMERVEWLRKKLQDVHNFVALGAPLAPRDAGSQRPRKKEDNV

SEQ ID NO: 28 - protein, 57 aa, synthetic construct
  REGION 1..57: note = human PTH 1-57
SVSEIQLMHNLGKHLNSMERVEWLRKKLQDVHNFVALGAPLAPRDAGSQRPRKKEDN

SEQ ID NO: 29 - protein, 56 aa, synthetic construct
  REGION 1..56: note = human PTH 1-56
SVSEIQLMHNLGKHLNSMERVEWLRKKLQDVHNFVALGAPLAPRDAGSQRPRKKED

SEQ ID NO: 30 - protein, 55 aa, synthetic construct
  REGION 1..55: note = human PTH 1-55
SVSEIQLMHNLGKHLNSMERVEWLRKKLQDVHNFVALGAPLAPRDAGSQRPRKKE

SEQ ID NO: 31 - protein, 54 aa, synthetic construct
  REGION 1..54: note = human PTH 1-54
SVSEIQLMHNLGKHLNSMERVEWLRKKLQDVHNFVALGAPLAPRDAGSQRPRKK

SEQ ID NO: 32 - protein, 53 aa, synthetic construct
  REGION 1..53: note = human PTH 1-53
SVSEIQLMHNLGKHLNSMERVEWLRKKLQDVHNFVALGAPLAPRDAGSQRPRK

SEQ ID NO: 33 - protein, 52 aa, synthetic construct
  REGION 1..52: note = human PTH 1-52
SVSEIQLMHNLGKHLNSMERVEWLRKKLQDVHNFVALGAPLAPRDAGSQRPR

SEQ ID NO: 34 - protein, 51 aa, synthetic construct
  REGION 1..51: note = human PTH 1-51
SVSEIQLMHNLGKHLNSMERVEWLRKKLQDVHNFVALGAPLAPRDAGSQRP

SEQ ID NO: 35 - protein, 50 aa, synthetic construct
  REGION 1..50: note = human PTH 1-50
SVSEIQLMHNLGKHLNSMERVEWLRKKLQDVHNFVALGAPLAPRDAGSQR

SEQ ID NO: 36 - protein, 49 aa, synthetic construct
  REGION 1..49: note = human PTH 1-49
SVSEIQLMHNLGKHLNSMERVEWLRKKLQDVHNFVALGAPLAPRDAGSQ

SEQ ID NO: 37 - protein, 48 aa, synthetic construct
  REGION 1..48: note = human PTH 1-48
SVSEIQLMHNLGKHLNSMERVEWLRKKLQDVHNFVALGAPLAPRDAGS

SEQ ID NO: 38 - protein, 47 aa, synthetic construct
  REGION 1..47: note = human PTH 1-47
SVSEIQLMHNLGKHLNSMERVEWLRKKLQDVHNFVALGAPLAPRDAG

SEQ ID NO: 39 - protein, 46 aa, synthetic construct
  REGION 1..46: note = human PTH 1-46
SVSEIQLMHNLGKHLNSMERVEWLRKKLQDVHNFVALGAPLAPRDA

SEQ ID NO: 40 - protein, 45 aa, synthetic construct
  REGION 1..45: note = human PTH 1-45
SVSEIQLMHNLGKHLNSMERVEWLRKKLQDVHNFVALGAPLAPRD

SEQ ID NO: 41 - protein, 44 aa, synthetic construct
  REGION 1..44: note = human PTH 1-44
SVSEIQLMHNLGKHLNSMERVEWLRKKLQDVHNFVALGAPLAPR

SEQ ID NO: 42 - protein, 43 aa, synthetic construct
  REGION 1..43: note = human PTH 1-43
SVSEIQLMHNLGKHLNSMERVEWLRKKLQDVHNFVALGAPLAP

SEQ ID NO: 43 - protein, 42 aa, synthetic construct
  REGION 1..42: note = human PTH 1-42
SVSEIQLMHNLGKHLNSMERVEWLRKKLQDVHNFVALGAPLA

SEQ ID NO: 44 - protein, 41 aa, synthetic construct
  REGION 1..41: note = human PTH-41
SVSEIQLMHNLGKHLNSMERVEWLRKKLQDVHNFVALGAPL

SEQ ID NO: 45 - protein, 40 aa, synthetic construct
  REGION 1..40: note = human PTH 1-40
SVSEIQLMHNLGKHLNSMERVEWLRKKLQDVHNFVALGAP

SEQ ID NO: 46 - protein, 39 aa, synthetic construct
  REGION 1..39: note = human PTH 1-39
SVSEIQLMHNLGKHLNSMERVEWLRKKLQDVHNFVALGA

SEQ ID NO: 47 - protein, 38 aa, synthetic construct
  REGION 1..38: note = human PTH 1-38
SVSEIQLMHNLGKHLNSMERVEWLRKKLQDVHNFVALG

SEQ ID NO: 48 - protein, 37 aa, synthetic construct
  REGION 1..37: note = human PTH 1-37
SVSEIQLMHNLGKHLNSMERVEWLRKKLQDVHNFVAL

SEQ ID NO: 49 - protein, 36 aa, synthetic construct
  REGION 1..36: note = human PTH 1-36
SVSEIQLMHNLGKHLNSMERVEWLRKKLQDVHNFVA

SEQ ID NO: 50 - protein, 35 aa, synthetic construct
  REGION 1..35: note = human PTH 1-35
SVSEIQLMHNLGKHLNSMERVEWLRKKLQDVHNFV

SEQ ID NO: 51 - protein, 34 aa, synthetic construct
  REGION 1..34: note = human PTH 1-34
SVSEIQLMHNLGKHLNSMERVEWLRKKLQDVHNF

SEQ ID NO: 52 - protein, 33 aa, synthetic construct
  REGION 1..33: note = human PTH 1-33
SVSEIQLMHNLGKHLNSMERVEWLRKKLQDVHN

SEQ ID NO: 53 - protein, 32 aa, synthetic construct
  REGION 1..32: note = human PTH 1-32
SVSEIQLMHNLGKHLNSMERVEWLRKKLQDVH

SEQ ID NO: 54 - protein, 31 aa, synthetic construct
  REGION 1..31: note = human PTH 1-31
SVSEIQLMHNLGKHLNSMERVEWLRKKLQDV

SEQ ID NO: 55 - protein, 30 aa, synthetic construct
  REGION 1..30: note = human PTH 1-30
SVSEIQLMHNLGKHLNSMERVEWLRKKLQD

SEQ ID NO: 56 - protein, 29 aa, synthetic construct
  REGION 1..29: note = human PTH 1-29
SVSEIQLMHNLGKHLNSMERVEWLRKKLQ

SEQ ID NO: 57 - protein, 28 aa, synthetic construct
  REGION 1..28: note = human PTH 1-28
SVSEIQLMHNLGKHLNSMERVEWLRKKL

SEQ ID NO: 58 - protein, 27 aa, synthetic construct
  REGION 1..27: note = human PTH 1-27
SVSEIQLMHNLGKHLNSMERVEWLRKK

SEQ ID NO: 59 - protein, 26 aa, synthetic construct
  REGION 1..26: note = human PTH 1-26
SVSEIQLMHNLGKHLNSMERVEWLRK

SEQ ID NO: 60 - protein, 25 aa, synthetic construct
  REGION 1..25: note = human PTH 1-25
SVSEIQLMHNLGKHLNSMERVEWLR

SEQ ID NO: 61 - protein, 84 aa, synthetic construct; identical to SEQ ID NO: 1
  REGION 1..84: note = amidated human PTH 1-84
  MOD_RES 84: note = AMIDATION

SEQ ID NO: 62 - protein, 83 aa, synthetic construct; identical to SEQ ID NO: 2
  REGION 1..83: note = amidated human PTH 1-83
  MOD_RES 83: note = AMIDATION

SEQ ID NO: 63 - protein, 82 aa, synthetic construct; identical to SEQ ID NO: 3
  REGION 1..82: note = amidated human PTH 1-82
  MOD_RES 82: note = AMIDATION

SEQ ID NO: 64 - protein, 81 aa, synthetic construct; identical to SEQ ID NO: 4
  REGION 1..81: note = amidated human PTH 1-81
  MOD_RES 81: note = AMIDATION

SEQ ID NO: 65 - protein, 80 aa, synthetic construct; identical to SEQ ID NO: 5
  REGION 1..80: note = amidated human PTH 1-80
  MOD_RES 80: note = AMIDATION

SEQ ID NO: 66 - protein, 79 aa, synthetic construct; identical to SEQ ID NO: 6
  REGION 1..79: note = amidated human PTH 1-79
  MOD_RES 79: note = AMIDATION

SEQ ID NO: 67 - protein, 78 aa, synthetic construct; identical to SEQ ID NO: 7
  REGION 1..78: note = amidated human PTH 1-78
  MOD_RES 78: note = AMIDATION

SEQ ID NO: 68 - protein, 77 aa, synthetic construct; identical to SEQ ID NO: 8
  REGION 1..77: note = amidated human PTH 1-77
  MOD_RES 77: note = AMIDATION

SEQ ID NO: 69 - protein, 76 aa, synthetic construct; identical to SEQ ID NO: 9
  REGION 1..76: note = amidated human PTH 1-76
  MOD_RES 76: note = AMIDATION

SEQ ID NO: 70 - protein, 75 aa, synthetic construct; identical to SEQ ID NO: 10
  REGION 1..75: note = amidated human PTH 1-75
  MOD_RES 75: note = AMIDATION

SEQ ID NO: 71 - protein, 74 aa, synthetic construct; identical to SEQ ID NO: 11
  REGION 1..74: note = amidated human PTH 1-74
  MOD_RES 74: note = AMIDATION

SEQ ID NO: 72 - protein, 73 aa, synthetic construct; identical to SEQ ID NO: 12
  REGION 1..73: note = amidated human PTH 1-73
  MOD_RES 73: note = AMIDATION

SEQ ID NO: 73 - protein, 72 aa, synthetic construct; identical to SEQ ID NO: 13
  REGION 1..72: note = amidated human PTH 1-72
  MOD_RES 72: note = AMIDATION

SEQ ID NO: 74 - protein, 71 aa, synthetic construct; identical to SEQ ID NO: 14
  REGION 1..71: note = amidated human PTH 1-71
  MOD_RES 71: note = AMIDATION

SEQ ID NO: 75 - protein, 70 aa, synthetic construct; identical to SEQ ID NO: 15
  REGION 1..70: note = amidated human PTH 1-70
  MOD_RES 70: note = AMIDATION

SEQ ID NO: 76 - protein, 69 aa, synthetic construct; identical to SEQ ID NO: 16
  REGION 1..69: note = amidated human PTH 1-69
  MOD_RES 69: note = AMIDATION

SEQ ID NO: 77 - protein, 68 aa, synthetic construct; identical to SEQ ID NO: 17
  REGION 1..68: note = amidated human PTH 1-68
  MOD_RES 68: note = AMIDATION

SEQ ID NO: 78 - protein, 67 aa, synthetic construct; identical to SEQ ID NO: 18
  REGION 1..67: note = amidated human PTH 1-67
  MOD_RES 67: note = AMIDATION

SEQ ID NO: 79 - protein, 66 aa, synthetic construct; identical to SEQ ID NO: 19
  REGION 1..66: note = amidated human PTH 1-66
  MOD_RES 66: note = AMIDATION

SEQ ID NO: 80 - protein, 65 aa, synthetic construct; identical to SEQ ID NO: 20
  REGION 1..65: note = amidated human PTH 1-65
  MOD_RES 65: note = AMIDATION

SEQ ID NO: 81 - protein, 64 aa, synthetic construct; identical to SEQ ID NO: 21
  REGION 1..64: note = amidated human PTH 1-64
  MOD_RES 64: note = AMIDATION

SEQ ID NO: 82 - protein, 63 aa, synthetic construct; identical to SEQ ID NO: 22
  REGION 1..63: note = amidated human PTH 1-63
  MOD_RES 63: note = AMIDATION

SEQ ID NO: 83 - protein, 62 aa, synthetic construct; identical to SEQ ID NO: 23
  REGION 1..62: note = amidated human PTH 1-62
  MOD_RES 62: note = AMIDATION

SEQ ID NO: 84 - protein, 61 aa, synthetic construct; identical to SEQ ID NO: 24
  REGION 1..61: note = amidated human PTH 1-61
  MOD_RES 61: note = AMIDATION

SEQ ID NO: 85 - protein, 60 aa, synthetic construct; identical to SEQ ID NO: 25
  REGION 1..60: note = amidated human PTH 1-60
  MOD_RES 60: note = ACETYLATION

SEQ ID NO: 86 - protein, 59 aa, synthetic construct; identical to SEQ ID NO: 26
  REGION 1..59: note = amidated human PTH 1-59
  MOD_RES 59: note = AMIDATION

SEQ ID NO: 87 - protein, 58 aa, synthetic construct; identical to SEQ ID NO: 27
  REGION 1..58: note = amidated human PTH 1-58
  MOD_RES 58: note = AMIDATION

SEQ ID NO: 88 - protein, 57 aa, synthetic construct; identical to SEQ ID NO: 28
  REGION 1..57: note = amidated human PTH 1-57
  MOD_RES 57: note = AMIDATION

SEQ ID NO: 89 - protein, 56 aa, synthetic construct; identical to SEQ ID NO: 29
  REGION 1..56: note = amidated human PTH 1-56
  MOD_RES 56: note = AMIDATION

SEQ ID NO: 90 - protein, 55 aa, synthetic construct; identical to SEQ ID NO: 30
  REGION 1..55: note = amidated human PTH 1-55
  MOD_RES 55: note = AMIDATION

SEQ ID NO: 91 - protein, 54 aa, synthetic construct; identical to SEQ ID NO: 31
  REGION 1..54: note = amidated human PTH 1-54
  MOD_RES 54: note = AMIDATION

SEQ ID NO: 92 - protein, 53 aa, synthetic construct; identical to SEQ ID NO: 32
  REGION 1..53: note = amidated human PTH 1-53
  MOD_RES 53: note = AMIDATION

SEQ ID NO: 93 - protein, 52 aa, synthetic construct; identical to SEQ ID NO: 33
  REGION 1..52: note = amidated human PTH 1-52
  MOD_RES 52: note = AMIDATION

SEQ ID NO: 94 - protein, 51 aa, synthetic construct; identical to SEQ ID NO: 34
  REGION 1..51: note = amidated human PTH 1-51
  MOD_RES 51: note = AMIDATION

SEQ ID NO: 95 - protein, 50 aa, synthetic construct; identical to SEQ ID NO: 35
  REGION 1..50: note = amidated human PTH 1-50
  MOD_RES 50: note = AMIDATION

SEQ ID NO: 96 - protein, 49 aa, synthetic construct; identical to SEQ ID NO: 36
  REGION 1..49: note = amidated human PTH 1-49
  MOD_RES 49: note = AMIDATION

SEQ ID NO: 97 - protein, 48 aa, synthetic construct; identical to SEQ ID NO: 37
  REGION 1..48: note = amidated human PTH 1-48
  MOD_RES 48: note = AMIDATION

SEQ ID NO: 98 - protein, 47 aa, synthetic construct; identical to SEQ ID NO: 38
  REGION 1..47: note = amidated human PTH 1-47
  MOD_RES 47: note = AMIDATION

SEQ ID NO: 99 - protein, 46 aa, synthetic construct; identical to SEQ ID NO: 39
  REGION 1..46: note = amidated human PTH 1-46
  MOD_RES 46: note = AMIDATION

SEQ ID NO: 100 - protein, 45 aa, synthetic construct; identical to SEQ ID NO: 40
  REGION 1..45: note = amidated human PTH 1-45
  MOD_RES 45: note = AMIDATION

SEQ ID NO: 101 - protein, 44 aa, synthetic construct; identical to SEQ ID NO: 41
  REGION 1..44: note = amidated human PTH 1-44
  MOD_RES 44: note = AMIDATION

SEQ ID NO: 102 - protein, 43 aa, synthetic construct; identical to SEQ ID NO: 42
  REGION 1..43: note = amidated human PTH 1-43
  MOD_RES 43: note = AMIDATION

SEQ ID NO: 103 - protein, 42 aa, synthetic construct; identical to SEQ ID NO: 43
  REGION 1..42: note = amidated human PTH 1-42
  MOD_RES 42: note = AMIDATION

SEQ ID NO: 104 - protein, 41 aa, synthetic construct; identical to SEQ ID NO: 44
  REGION 1..41: note = amidated human PTH 1-41
  MOD_RES 41: note = AMIDATION

SEQ ID NO: 105 - protein, 40 aa, synthetic construct; identical to SEQ ID NO: 45
  REGION 1..40: note = amidated human PTH 1-40
  MOD_RES 40: note = AMIDATION

SEQ ID NO: 106 - protein, 39 aa, synthetic construct; identical to SEQ ID NO: 46
  REGION 1..39: note = amidated human PTH 1-39
  MOD_RES 39: note = AMIDATION

SEQ ID NO: 107 - protein, 38 aa, synthetic construct; identical to SEQ ID NO: 47
  REGION 1..38: note = amidated human PTH 1-38
  MOD_RES 38: note = AMIDATION

SEQ ID NO: 108 - protein, 37 aa, synthetic construct; identical to SEQ ID NO: 48
  REGION 1..37: note = amidated human PTH 1-37
  MOD_RES 37: note = AMIDATION

SEQ ID NO: 109 - protein, 36 aa, synthetic construct; identical to SEQ ID NO: 49
  REGION 1..36: note = amidated human PTH 1-36
  MOD_RES 36: note = AMIDATION

SEQ ID NO: 110 - protein, 35 aa, synthetic construct; identical to SEQ ID NO: 50
  REGION 1..35: note = amidated human PTH 1-35
  MOD_RES 35: note = AMIDATION

SEQ ID NO: 111 - protein, 34 aa, synthetic construct; identical to SEQ ID NO: 51
  REGION 1..34: note = amidated human PTH 1-34
  MOD_RES 34: note = AMIDATION

SEQ ID NO: 112 - protein, 33 aa, synthetic construct; identical to SEQ ID NO: 52
  REGION 1..33: note = amidated human PTH 1-33
  MOD_RES 33: note = AMIDATION

SEQ ID NO: 113 - protein, 32 aa, synthetic construct; identical to SEQ ID NO: 53
  REGION 1..32: note = amidated human PTH 1-32
  MOD_RES 32: note = AMIDATION

SEQ ID NO: 114 - protein, 31 aa, synthetic construct; identical to SEQ ID NO: 54
  REGION 1..31: note = amidated human PTH 1-31
  MOD_RES 31: note = AMIDATION

SEQ ID NO: 115 - protein, 30 aa, synthetic construct; identical to SEQ ID NO: 55
  REGION 1..30: note = amidated human PTH 1-30
  MOD_RES 30: note = AMIDATION

SEQ ID NO: 116 - protein, 29 aa, synthetic construct
  REGION 1..29: note = amidated human PTH 1-29
  MOD_RES 29: note = AMIDATION
SVSEIQLMHNLGKHLNSMERVEWLRKKLQ

SEQ ID NO: 117 - protein, 28 aa, synthetic construct
  REGION 1..28: note = amidated human PTH 1-28
  MOD_RES 28: note = AMIDATION
SVSEIQLMHNLGKHLNSMERVEWLRKKL

SEQ ID NO: 118 - protein, 27 aa, synthetic construct
  REGION 1..27: note = amidated human PTH 1-27
  MOD_RES 27: note = AMIDATION
SVSEIQLMHNLGKHLNSMERVEWLRKK

SEQ ID NO: 119 - protein, 26 aa, synthetic construct
  REGION 1..26: note = amidated human PTH 1-26
  MOD_RES 26: note = AMIDATION
SVSEIQLMHNLGKHLNSMERVEWLRK

SEQ ID NO: 120 - protein, 25 aa, synthetic construct
  REGION 1..25: note = amidated human PTH 1-25
  MOD_RES 25: note = AMIDATION
SVSEIQLMHNLGKHLNSMERVEWLR

SEQ ID NO: 121 - protein, 141 aa, Homo sapiens
AVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 122 - protein, 36 aa, synthetic construct
  REGION 1..36: note = Artificial random coil
AVAEIQLMHQRAKWIQDARRRAFLHKLIAEIHTAEI

SEQ ID NO: 123 - protein, 32 aa, synthetic construct
  VARIANT 10: note = X= Asp, Gln or Asn
  VARIANT 13: note = X = Arg, Glu, Asp or Lys
  VARIANT 16: note = X= Asp, Gln or Asn
  VARIANT 18: note = X = Met, Met(O), Leu or Nleu
  VARIANT 26: note = X = Arg, Glu, Asp or Lys
  VARIANT 27: note = X = Arg, Glu, Asp or Lys
  VARIANT 31: note = X = Gly or Val
SVSEIQLMHXLGXHLXSXERVEWLRXXLQDXH

SEQ ID NO: 124 - protein, 32 aa, synthetic construct
  VARIANT 10: note = X = Asp, Gln or Asn
  VARIANT 12: note = X = Aib or Gly
  VARIANT 16: note = X =Aib or Asn
  VARIANT 17: note = X = Aib or Ser
  VARIANT 18: note = X = Met, Met(O), Leu or Nleu
SVSEIQLMHXLXKHLXXXERVEWLRKKLQDVH

SEQ ID NO: 125 - protein, 32 aa, synthetic construct
  VARIANT 10: note = X = Asp, Gln or Asn
  VARIANT 16: note = X = Asp, Gln or Asn
  VARIANT 18: note = X = Met, Met(O), Leu or Nleu
SVSEIQLMHXLGKHLXSXERVEWLRKKLQDVH

SEQ ID NO: 126 - protein, 32 aa, synthetic construct; identical to SEQ ID NO: 53

SEQ ID NO: 127 - protein, 6 aa, synthetic construct
  SITE 1: note = acylated amino acid comprising a C16-C30 fatty acid or C16-C30 diacid covalently linked to the side chain of the amino acid
  SITE 6: note = acylated amino acid comprising a C16-C30 fatty acid or C16-C30 diacid covalently linked to the side chain of the amino acid
XFVALX

SEQ ID NO: 128 - protein, 7 aa, synthetic construct
  SITE 1: note = acylated amino acid comprising a C16-C30 fatty acid or C16-C30 diacid covalently linked to the side chain of the amino acid
  SITE 6: note = acylated amino acid comprising a C16-C30 fatty acid or C16-C30 diacid covalently linked to the side chain of the amino acid
XFVALGX

SEQ ID NO: 129 - protein, 6 aa, synthetic construct
  VARIANT 1: note = X = Gln or Asn
  SITE 6: note = acylated amino acid comprising a C16-C30 fatty acid or C16-C30 diacid covalently linked to the side chain of the amino acid
XFVALX

SEQ ID NO: 130 - protein, 7 aa, synthetic construct
  VARIANT 1: note = X = Gln or Asn
  SITE 7: note = acylated amino acid comprising a C16-C30 fatty acid or C16-C30 diacid covalently linked to the side chain of the amino acid
XFVALGX

SEQ ID NO: 131 - protein, 33 aa, synthetic construct
  SITE 33: note = amino acid comprising a C16-C30 fatty acid or C16-C30 diacid covalently linked to the acid side chain
SVSEIQLMHNLGKHLNSMERVEWLRKKLQDVHX

SEQ ID NO: 132 - protein, 35 aa, synthetic construct
  SITE 35: note = amino acid comprising a C16-C30 fatty acid or C16-C30 diacid covalently linked to the acid side chain
SVSEIQLMHNLGKHLNSMERVEWLRKKLQDVHNFX

SEQ ID NO: 133 - protein, 32 aa, synthetic construct
  VARIANT 13: note = X = Arg, Glu, Asp or Lys
  VARIANT 26: note = X = Arg, Glu, Asp or Lys
  VARIANT 27: note = X = Arg, Glu, Asp or Lys
  VARIANT 31: note = X = Gly or Val
SVSEIQLMHNLGXHLNSMERVEWLRXXLQDXH

SEQ ID NO: 134 - protein, 32 aa, synthetic construct; identical to SEQ ID NO: 125
  VARIANT 10: note = X =Asp, Gln or Asn
  VARIANT 16: note = X =Asp, Gln or Asn
  VARIANT 18: note = X= Met, Met(O), Leu or Nleu